(19) United States
(12) Patent Application Publication
(10) Pub. No.: US 2024/0277632 A1
(43) Pub. Date: Aug. 22, 2024

(54) Epinephrine Pharmaceutical Formulations for Intranasal Delivery

(71) Applicant: Amphastar Pharmaceuticals, Inc., Rancho Cucamonga, CA (US)

(72) Inventors: Jack Yongfeng Zhang, Rancho Cucamonga, CA (US); Mary Zi-ping Luo, Rancho Cucamonga, CA (US); Jie Fei Ding, Rancho Cucamonga, CA (US); Aili Bo, Rancho Cucamonga, CA (US)

(21) Appl. No.: 17/917,541

(22) PCT Filed: May 3, 2021
(86) PCT No.: PCT/US21/30502

§ 371 Date: Oct. 6, 2022

Related U.S. Application Data
(60) Provisional application No. 63/020,002, filed on May 4, 2020.

(51) Int. Cl. A61K 31/137; A61K 9/00; A61K 47/10; A61K 47/12; A61K 47/18; A61K 47/28

(52) U.S. Cl. CPC A61K 31/137; A61K 9/0043; A61K 47/10; A61K 47/12; A61K 47/183; A61K 47/28

(57) ABSTRACT

The present disclosure introduces pharmaceutical formulations having epinephrine as the active pharmaceutical ingredient (API), and a bile acid, or salt thereof (e.g., sodium taurocholate (STC)) as an absorption enhancer, for intranasal (IN) delivery. The bile acid, or the salt thereof, enhances absorption of epinephrine by IN delivery. Also disclosed are methods of administering epinephrine to a patient by IN delivery using the disclosed epinephrine formulations for various treatments or indications.

### CROSS REFERENCE TO RELATED APPLICATIONS

The present application claims the benefit of priority to U.S. Provisional Patent Application No. 63/020,002, filed May 4, 2020, the entirety of which is hereby incorporated by reference herein.

### FIELD

The present disclosure generally pertains to pharmaceutical formulations suitable for and/or configured for intranasal (IN) delivery, methods of manufacturing such formulations, and methods of treating patients using such formulations.

### BACKGROUND

### Epinephrine Background

Epinephrine is the active principle of the adrenal medulla and is chemically described as (-)-3,4-Dihy droxy-α-[(methyl-amino) methyl]benzyl alcohol. Its molecular formula is C₉H₁₃NO₃, and it has a molecular weight (M.W.) of 183.2 g/mol. Epinephrine is also commonly known as adrenaline or catecholamine. Epinephrine has the following structural formula:

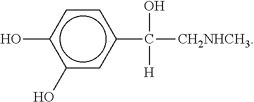

Epinephrine is a non-selective alpha and beta adrenergic agonist commonly used for a variety of indications, particularly as an emergency treatment. For example, epinephrine is indicated to treat allergic reactions (e.g. anaphylaxis), to increase mean arterial blood pressure in patients with hypotension associated with septic shock, to relieve respiratory distress due to bronchospasm, to provide rapid relief of hypersensitivity reactions to drugs and other allergens, to prolong the action of infiltration anesthetics, and other indications. In fact, epinephrine is often the drug of choice in the treatment of anaphylaxis.

### SUMMARY

### An Unmet Need for Non-invasive Epinephrine Medicament.

Currently, pre-filled syringes of epinephrine injection (e.g., auto-injector) are given intramuscularly (IM) or subcutaneously. These routes are the ones approved for first-aid treatment of anaphylaxis using epinephrine. Unfortunately, auto-injectors for epinephrine have several inherent limitations in their clinical use. For example, although anaphylaxis is a life-threatening emergency, it takes several steps to correctly operate an Epi-pen® auto-injector, which is indicated to treat anaphylaxis, to administer the treatment. Moreover, injection is invasive, often painful, can be feared by the individual receiving the inject and, most importantly, incorrect or unintentional operation could cause injury to a patients or subject. All of these factors are limitations of Epi-pen® auto-injectors. Therefore, an unmet medical need exists to develop epinephrine drug products with an alternative delivery route to overcome these limitations (or others) that are associated with injections or auto-injectors. A potential alternative to injection delivery is to deliver the drug through the nasal cavity, also referred to as intranasal (IN) delivery herein.

As disclosed elsewhere herein, the IN route of administration is a non-invasive drug delivery method that can provide a rapid onset of drug action. This route also offers several practical advantages over IM injection. For instance, the IN administration is easy to perform, painless, and can be performed by the patient (e.g., they are self-administrable) without using needles. Additionally, IN delivery devices are easy to carry and easier to operate than devices for injection. Moreover, medicine for treatment of anaphylaxis (e.g., epinephrine) is typically administered in emergencies. In this regard, an added advantage of IN epinephrine delivery, on top of the easy-to-handle features, is an improved rapid onset of drug action (e.g., onset that meets or exceeds that of IM administration). To date, no intranasal epinephrine formulation that achieves these goals is available on the market.

### Pharmacological Challenge in Intranasal Epinephrine Delivery.

Epinephrine is highly hydrophilic and has low membrane permeability, rendering it difficult to penetrate the upper epithelial layer of the nasal mucosa, which is predominantly lipophilic. Thus, when aqueous epinephrine is delivered by the IN route without an enhancing agent, the absorption can be either slow or result in minimal bioavailability. Consequently, it poses a pharmacological challenge to improve epinephrine absorption through the nasal mucosa.

The present disclosure addresses these technical challenges, or others, by providing pharmaceutical formulations comprising an absorption enhancer, to increase drug absorption through the upper epithelial layer, which is predominantly lipophilic. The choice of enhancers is important, because each candidate enhancer can have its own unique physiochemical interactions with the active pharmaceutical ingredient (e.g., epinephrine) as well as the mucosa. As natural amphipathic molecules, bile acids and salts thereof have both hydrophobic and hydrophilic regions. Bile acids and salts thereof can also form micelles above a certain concentration. Without being bound to any particular theory, it is believed that the addition of bile acids or salts thereof to the formulations disclosed herein change properties at the lipid-water interface, thereby helping epinephrine travel through the upper epithelial layer in the nasal mucosa. In addition, it has now been found that, contrary to prior teachings, bile acids and salts thereof are well in the disclosed formulations. Bile acids and salts thereof are absorbed and excreted easily and generally regarded as safe according to FDA's GRAS Substances (SCOGS) database.

Disclosed are pharmaceuticals formulations suitable for intranasal (IN) delivery. In several embodiments, the formulations include epinephrine as the active pharmaceutical ingredient (API), and a bile acid, or a salt thereof (e.g., sodium taurocholate (STC)). In several embodiments, the bile acid or salt thereof acts as the absorption enhancer for enhancing the absorption of epinephrine into a human subject's bloodstream by IN delivery. Also disclosed are methods of providing a rapid delivery of epinephrine to a patient by IN delivery using the disclosed epinephrine formulations for various treatments, or indications.

In several embodiments, the pharmaceutical formulation comprises epinephrine, or a pharmaceutically acceptable salt as the active pharmaceutical ingredient (API). thereof at a concentration ranging from 1.0 mg/ml to 25.0 mg/ml. In other embodiments, the concentration ranges from 6.0 mg/ml to 10.0 mg/ml. In several embodiments, the concentration ranges from 10.0 mg/ml to 14.0 mg/ml. In several embodiments, the therapeutically effective amount of epinephrine is suitable for the treatment of a type-I hypersensitivity reaction.

In several embodiments, the pharmaceutical formulation comprises one or more absorption enhancers. In several embodiments, the one or more absorption enhancers comprise, consist of, or consist essentially of one or more bile acids or bile acid salts. In several embodiments, the bile acid or bile acid salt is present in the formulation at a concentration ranging from 1.0 mg/ml to 15 mg/ml. In several embodiments, the bile acid or bile acid salt is present in the formulation at a concentration ranging from 7.0 mg/ml to 9.0 mg/ml. In several embodiments, the bile acid or bile acid salt is present in the formulation at a concentration ranging from 8.0 mg/ml to 12.0 mg/ml.

In several embodiments, the bile acid, or the salt thereof, is a trihydroxy conjugate, including, but not limited to glycocholate (GC), taurocholate (TC), glycohyocholate (GHC), taurohyocholate (THC), tauro-α-hyocholate (T-α-MC), tauro-β-hyocholate (T-β-MC) or a combination of any of the foregoing. These trihydroxy conjugates may be in their acid or salt forms (e.g., sodium salt form).

In several embodiments, the bile acid, or the salt thereof, is a dihydroxy conjugate, including, but not limited to taurohyodeoxycholate (THDC), glycohyodeoxycholate (GHDC), glycochenodeoxycholate (GCDC), taurodeoxycholate (TDC), taurochenodeoxycholate (TCDC), glycodeoxychoate (GDC), glycoursodeoxycholate (GUDC), or a combination of any of the foregoing. These dihydroxy conjugates may be in their acid or salt forms (e.g., sodium salt form).

In several embodiments, the bile acid, or the salt thereof, is an unconjugated form. In several embodiments, the bile acid or salt thereof comprises, consists essentially of, or consists of cholate, deoxycholate (DC), chenodeoxycholate (CDC), or a combination of any of the foregoing. These unconjugated forms may be in their acid or salt forms (e.g., sodium salt form).

In several embodiments, the one or more absorption enhancers comprises a combination of bile acids and/or bile acid salts. In several embodiments, only a single absorption enhancer is used. In several embodiments, the absorption enhancer comprises, consists essentially of, or consists of a taurocholate salt. In several embodiments, the absorption enhancer comprises, consists essentially of, or consists of sodium taurocholate. In several embodiments, the absorption enhancer comprises, consists essentially of, or consists of taurocholic acid.

In several embodiments, the bile salt comprises, consists essentially of, or consists of sodium taurocholate (STC). In several embodiments, the STC is present at a concentration of 1.0 mg/mL to 15.0 mg/mL. In several embodiments, the STC is present at a concentration of 8.0 mg/mL to 12.0 mg/mL. In several embodiments, the STC is present at a concentration of 9.0 mg/mL to 11.0 mg/mL. In several embodiments, the STC is present at a concentration of 10.0 mg/mL. In several embodiments, the STC is present in a dose amount of 0.1 mg to 1.5 mg. In several embodiments, the STC is present at a concentration of 5.0 mg/mL to 11.0 mg/mL. In several embodiments, the STC is present at a concentration of 7.0 mg/mL to 9.0 mg/mL.

In several embodiments, the pharmaceutical formulation (e.g., composition) comprises a buffer. In several embodiments, the buffer comprises, consists essentially of, or consists of a citrate buffer. In several embodiments, the pharmaceutical composition comprises a citric acid source at a concentration that ranges from 2 mg/ml to 6 mg/ml. In several embodiments, the citric acid source is citric acid monohydrate. In several embodiments, the pharmaceutical composition comprises a sodium citrate source at a concentration that ranges from 6 mg/ml to 10 mg/ml. In several embodiments, the sodium citrate source is sodium citrate dihydrate. In several embodiments, the pH of the pharmaceutical formulation ranges from 2.6 to 5.0. In several embodiments, the pH of the pharmaceutical formulation ranges from 3.6 to 4.0.

In several embodiments, the pharmaceutical composition comprises a preservative. In several embodiments, the preservative comprises, consists essentially of, or consists of a chlorbutanol source. In several embodiments, the chlorbutanol source is chlorbutanol hemihydrate. In several embodiments, the preservative is at a concentration that ranges from 3.5 mg/ml to 7.5 mg/ml.

In several embodiments, the pharmaceutical composition comprises a tonicity agent. In several embodiments, the tonicity agent comprises, consists essentially of, or consists of sodium chloride. In several embodiments, the tonicity agent is at a concentration that ranges from 1 mg/ml to 3 mg/ml.

In several embodiments, the pharmaceutical composition comprises a metal complexing agent. In several embodiments, the metal complexing agent comprises, consists essentially of, or consists of an ethylenediaminetetraacetic acid (EDTA) source. In several embodiments, the EDTA source is disodium edetate dihydrate. In several embodiments, the metal complexing agent is at a concentration that ranges from 0.01 mg/ml to 0.03 mg/ml.

In several embodiments, the pharmaceutical composition comprises an antioxidant. In several embodiments, the metal complexing agent comprises, consists essentially of, or consists of sodium metabisulfite. In several embodiments, the antioxidant is at a concentration that ranges from 0.2 mg/ml to 0.4 mg/ml.

In several embodiments, the pharmaceutical composition has an osmolarity ranging from 200 mOsmol to 260 mOsmol.

In several embodiments, the pharmaceutical formulation comprises an aqueous carrier (e.g., water, saline).

Several embodiments pertain to a method of making the formulation. In several embodiments, the method comprises adding one or more buffering agents to the water. In several embodiments, the method comprises adjusting the pH to a range from 2.2 to 5.0. In several embodiments, the method comprises adding a preservative to the water. In several embodiments, the method comprises adding a tonicity agent to the water. In several embodiments, the method comprises adding an antioxidant to the water. In several embodiments, the method comprises adding a metal complexing agent to the water.

Several embodiments pertain to a pharmaceutical formulation configured for IN delivery. In several embodiments, the pharmaceutical formulation comprises, consists essentially of, or consists of 1.0 mg/mL to 25.0 mg/mL of epinephrine (or a pharmaceutically acceptable salt thereof), 1.0 mg/mL to 15.0 mg/mL of sodium taurocholate (STC), 1.0 mg/mL to 8.0 mg/mL of citric acid (e.g., citric acid, citric acid monohydrate, etc.), a buffer, 1.0 mg/mL to 4.0 mg/mL of sodium chloride, 0.01 mg/mL to 0.05 mg/mL of disodium edetate dihydrate, and 0.1 mg/mL to 1.0 mg/mL of sodium metabisulfite. In several embodiments, the pharmaceutical formulation has a pH of 2.2 to 5.0. In several embodiments, the pharmaceutical formulation is configured for IN delivery.

Several embodiments provide a pharmaceutical formulation comprising, consisting essentially of, or consisting of epinephrine at a concentration ranging from 6 mg/ml to 10 mg/ml, sodium taurocholate at a concentration ranging from 7 mg/ml to 9 mg/ml, sodium chloride at a concentration that ranges from 1 mg/ml to 3 mg/ml, a citrate buffer at molarity ranging from 0.050 molar to 0.075 molar, and water. Several embodiments provide a pharmaceutical formulation comprising, consisting essentially of, or consisting of epinephrine at a concentration ranging from 10 mg/ml to 14 mg/ml, sodium taurocholate at a concentration ranging from 8 mg/ml to 12 mg/ml, sodium chloride at a concentration that ranges from 1 mg/ml to 3 mg/ml, a citrate buffer at molarity ranging from 0.050 molar to 0.075 molar, and water. In several embodiments, the pharmaceutical formulation further comprises chlorbutanol at a concentration ranging from 3.5 mg/ml to 7.5 mg/ml. In several embodiments, the pharmaceutical formulation further comprises disodium edetate dihydrate at a concentration that ranges from 0.01 mg/ml to 0.03 mg/ml. In several embodiments, the pharmaceutical formulation further comprises sodium metabisulfite at a concentration that ranges from 0.2 mg/ml to 0.4 mg/ml. In several embodiments, the pharmaceutical formulation has a pH ranging from 3.6 to 4.0. In several embodiments, the pharmaceutical formulation has a pH ranging from 2.2 to 5.0. In several embodiments, the pharmaceutical formulation is configured to be administered intranasally.

Several embodiments pertain to a method of preparing a pharmaceutical formulation. In several embodiments, the method comprises dissolving epinephrine or a pharmaceutically acceptable salt thereof and an absorption enhancer in water. In several embodiments, the absorption enhancer consists of a bile acid or bile acid salt. In several embodiments, a final concentration of epinephrine or the pharmaceutically acceptable salt thereof in the pharmaceutical formulation ranges from 1 mg/ml to 25 mg/ml. In several embodiments, a final concentration of the absorption enhancer in the pharmaceutical formulation ranges from 1 mg/ml to 15 mg/ml. In several embodiments, the pharmaceutical formulation is configured to be administered intranasally.

In several embodiments, the method comprises adding one or more buffering agents to the water. In several embodiments, the method comprises adjusting the pH to a range from 2.2 to 5.0. In several embodiments, the method comprises adding a preservative to the water. In several embodiments, the method comprises adding a tonicity agent to the water. In several embodiments, the method comprises adding an antioxidant to the water. In several embodiments, the method comprises adding a metal complexing agent to the water.

In several embodiments, a single spray of the nasal spray discharges a dose volume of 0.05 mL to 0.15 mL of the pharmaceutical formulation. In several embodiments, the single spray of the nasal spray discharges a dose volume of about 0.10 mL of the pharmaceutical formulation. In several embodiments, the dose is delivered as an atomized spray.

In several embodiments, the pharmaceutical formulation is configured to be administered intranasally. In several embodiments, a 0.1 mL intranasal dose of the pharmaceutical formulation is configured to provide a therapeutically effective amount of epinephrine. In several embodiments, the therapeutically effective amount of epinephrine is suitable for the treatment of a type-I hypersensitivity reaction.

In several embodiments, the epinephrine, or the pharmaceutically acceptable salt thereof, is provided a dose amount at a range of 0.1 mg to 2.5 mg per single dose. In several embodiments, the epinephrine, or the pharmaceutically acceptable salt thereof, is provided in a dose amount at a range of 0.6 mg to 1.0 mg.

In several embodiments, bile acid, or the salt thereof, is provided a dose amount at a range of 1.0 mg to 1.5 mg. In several embodiments, the bile acid, or bile salt thereof, is provided in a dose amount at a range of 0.7 mg to 0.9 mg.

Also disclosed are methods of providing a rapid delivery of epinephrine to a patient by IN delivery using the disclosed epinephrine formulations for various treatments, or indications.

Some embodiments provide a method for rapid delivery of epinephrine to a human patient. In several embodiments, the method treats a condition of the patient. In several embodiments, the formulation is configured to provide and/or achieves an epinephrine Cₘₐₓ of 5 ng/mL to 15 ng/mL and a tₘₐₓ of less than 15 minutes. In several embodiments, the rapid delivery is via the intranasal route. In several embodiments, the method comprises administering a dose amount of epinephrine, or a pharmaceutically acceptable salt thereof, from a pharmaceutical formulation as disclosed above or elsewhere herein to at least one nostril of a human patient. In several embodiments, the method comprises administering the epinephrine intranasally. In several embodiments, the method comprises administering the epinephrine by intranasal (IN) delivery using a nasal spray.

In several embodiments, after administering the pharmaceutical formulation intranasally to the patient, a Cₘₐₓ equal for epinephrine in the patient is at least about 500 pg/mL. In several embodiments, after administering the pharmaceutical formulation intranasally to the patient, a tₘₐₓ for epinephrine in the patient is equal to or less than 10 minutes. In several embodiments, after administering the pharmaceutical formulation intranasally to the patient, an AUC_{0-t*} for epinephrine is equal to or at least about 25 pg/ml*hr. In several embodiments, after administering the pharmaceutical formulation intranasally to the patient, an AUC₀₋₃₀ₘᵢₙ for epinephrine is equal to or at least about 35 pg/ml*hr. In several embodiments, after administering the pharmaceutical formulation intranasally to the patient, an AUC₀₋₃₀ₘᵢₙ for epinephrine is equal to or at least about 110 pg/ml*hr.

Several embodiments pertain to a pharmaceutical formulation as disclosed above or elsewhere herein for use in treating a condition of a patient.

In several embodiments, after administering the pharmaceutical formulation intranasally to the patient, the formulation is configured to provide and/or achieves a tₘₐₓ of equal to or less than 12 minutes. In several embodiments, the formulation is configured to provide and/or achieves a tₘₐₓ of equal to or less than 10 minutes. In several embodiments, the formulation is configured to provide and/or achieves a tₘₐₓ of equal to or less than 5 minutes. In several embodiments, the formulation is configured to provide and/or achieves a tₘₐₓ of equal to or less than 3 minutes. In several embodiments, the formulation is configured to provide and/or achieves a Cₘₐₓ in a range of 10 ng/mL to 15 ng/mL. In several embodiments, the formulation is configured to provide and/or achieves a AUC_{0-10 min} in a range of 50 (ng*min)/mL to 80 (ng*min)/mL. In several embodiments, the formulation is configured to provide and/or achieves a AUC₀₋₃₀ₘᵢₙ in a range of 100 (ng*min)/mL to 170 (ng*min)/mL. In several embodiments, the formulation is configured to provide and/or achieves a AUC₀₋₁₈₀ₘᵢₙ in a range of 150 (ng*min)/mL to 300 (ng*min)/mL.

In several embodiments, the bile acid, or the salt thereof, provides an EF of at least 4, wherein the EF is determined based on [symbol] (S)/^{R̅}(0), where ^{R̅}(S) is an average of a dose-normalized relative bioavailability (DN-RBA) of the pharmaceutical formulation relative to an IM injection having same API for two or more pharmacokinetic (PK) parameters, and ^{R̅}(0) is an average of DN-RBA of a pharmaceutical formulation without an absorption enhancer relative to an IM injection having same API for the two or more PK parameters. In several embodiments, the EF is in a range of 4 to 23. In several embodiments, the PK parameters include AUC₀₋₃₀ₘᵢₙ, AUC₀₋₁₈₀ₘᵢₙ, and Cₘₐₓ.

In several embodiments, if the bile acid, or the salt thereof, causes decreased cilia in a respiratory epithelium of a subject (e.g., a human subject), then such decreased cilia is substantially reversed and/or resolved within 7 days. In several embodiments, if the bile acid, or the salt thereof, causes hyperplasia of a respiratory epithelium of a subject (e.g., a human subject), then such hyperplasia is substantially reversed and/or resolved within 7 days.

In several embodiments, no grade 2 or 3 events occur in the subject after a nasal and oropharyngeal mucosa examination (NOME). In several embodiments, no grade 3 events occur in the subject under self-reported nasal symptoms (SRNS) testing. In several embodiments, the subject experiences improved normosmia after the dose as measured by the University of Pennsylvania Smell Identification Test (UPSIT).

Several embodiments pertain to a method of treating a condition in a patient. In several embodiments, the method comprises a step of administering an intranasal dose of the pharmaceutical formulation as described above or elsewhere herein to at least a nostril of a human patient to treat a condition. In several embodiments, the condition is a type-I hypersensitivity reaction (systemic allergic reaction), an acute asthmatic attack, cardiac arrest, Stokes-Adams Syndrome, or a combination of the foregoing. In several embodiments, the condition is a type-I hypersensitivity reaction. In several embodiments, the type-I hypersensitivity reaction is selected from allergic asthma, allergic conjunctivitis, allergic rhinitis, anaphylaxis, angioedema, urticaria, eosinophilia, drug allergy, and food allergy. In several embodiments, the condition is an allergic reaction. In several embodiments, the condition is anaphylaxis. In several embodiments, the condition is hypotension associated with septic shock. In several embodiments, the pharmaceutical formulation is administered to increase mean arterial blood pressure in the patient. In several embodiments, the formulation comprises epinephrine, or a pharmaceutically acceptable salt thereof, and an absorption enhancer comprising a bile acid, or a salt thereof, and other pharmaceutically acceptable excipients. In several embodiments, the formulation has a pH of 2.2 to 5.0. In several embodiments, the bile acid, or the salt thereof, is a trihydroxy conjugate, a dihydroxy conjugate, an unconjugated form, or is a combination of the foregoing. In several embodiments, the bile salt is sodium taurocholate (STC).

In several embodiments, the epinephrine, or the pharmaceutically acceptable salt thereof, is present at a concentration of 1.0 mg/mL to 25.0 mg/mL, including any range subsumed therein (e.g., 8.0 mg/mL to 14 mg/mL, etc.). In several embodiments, the epinephrine, or the pharmaceutically acceptable salt thereof, is present in a dose amount of 0.1 mg to 2.5 mg, including any range subsumed therein. In several embodiments, the bile acid, or the salt thereof, is present at a concentration of 1.0 mg/mL to 15.0 mg/mL, including any range subsumed therein (e.g., 8.0 mg/mL to 12 mg/mL). In several embodiments, the bile acid, or the salt thereof, is present in a dose amount of 0.1 mg to 1.5 mg. Other exemplary embodiments of this disclosure will be detailed herein.

Several embodiments pertain to a pharmaceutical formulation for intranasal (IN) delivery comprising epinephrine, or a pharmaceutically acceptable salt thereof. In several embodiments, the pharmaceutical formulation comprises an absorption enhancer comprising a bile acid, or a salt thereof. In several embodiments, the absorption enhancer comprises, consists essentially of, or consists of a bile acid, or a salt thereof.

In several embodiments, the epinephrine, or the pharmaceutically acceptable salt thereof, is present at a concentration of 1.0 mg/mL to 30 mg/mL. In several embodiments, the epinephrine, or the pharmaceutically acceptable salt thereof, is present in a dose amount of 0.1 mg to 3 mg. In several embodiments, the epinephrine, or the pharmaceutically acceptable salt thereof, is present at a concentration of 2 mg/mL to 25 mg/mL. In several embodiments, the epinephrine, or the pharmaceutically acceptable salt thereof, is present in a dose amount of 0.2 mg to 2.5 mg. In several embodiments, the epinephrine, or the pharmaceutically acceptable salt thereof, is present at a concentration of 3 mg/mL to 20 mg/mL. In several embodiments, the epinephrine, or the pharmaceutically acceptable salt thereof, is present in a dose amount of 0.3 mg to 2 mg.

In several embodiments, as disclosed elsewhere herein, the bile acid or salt thereof is a trihydroxy conjugate. In several embodiments, the bile acid or salt thereof comprises, consists essentially of, or consists of glycocholate (GC), taurocholate (TC), glycolithocholate (GLC), glycohyocholate (GHC), glyco-7-oxo-lithocholate (G-7-oxo-LC), sodium glyco-7-oxo-lithocholate (SG-7-oxo-LC), taurolithocholate (TLC), taurohyocholate (THC), glycol-3α-6-keto-5β-cholate (glycol3α6keto-5β-cholate), tauro-α-hyocholate (T-α-MC), tauro-β-hyocholate (T-β-MC), or a combination of any of the foregoing.

In several embodiments, the bile acid salt comprises, consists essentially of, or consists of sodium glycocholate (SGC), sodium taurocholate (STC), sodium glycolithocholate (SGLC), sodium glycohyocholate (SGHC), sodium glyco-7-oxo-lithocholate (SG-7-oxo-LC), sodium taurolithocholate (STLC), sodium taurohyocholate (STHC), sodium glycol-3α-6-keto-5β-cholate (S-glycol3α6keto-5β-cholate), sodium tauro-α-hyocholate (S-T-α-MC), sodium tauro-p-hyocholate (S-T-β-MC), or a combination of any of the foregoing.

In several embodiments, the bile acid or salt thereof comprises, consists essentially of, or consists of a dihydroxy conjugate. In several embodiments, the bile acid or salt thereof comprises, consists essentially of, or consists of tauroursodeoxycholate (TUDC), taurohyodeoxycholate (THDC), glycohyodeoxycholate (GHDC), glycochenodeoxycholate (GCDC), taurodeoxycholate (TDC), taurochenodeoxycholate (TCDC), glycodeoxychoate (GDC), glycoursodeoxycholate (GUDC), or a combination thereof. In several embodiments, the bile acid salt comprises, consists essentially of, or consists of sodium tauroursodeoxycholate (STUDC), sodium taurohyodeoxycholate (STHDC), sodium glycohyodeoxycholate (SGHDC), sodium glycochenodeoxycholate (SGCDC), taurodeoxycholate (TDC), sodium taurodeoxycholate (STDC), sodium taurochenodeoxycholate (STCDC), sodium glycodeoxychoate (SGDC), sodium glycoursodeoxycholate (SGUDC), or a combination of any of the foregoing.

In several embodiments, the bile acid, or the salt thereof, is an unconjugated form. In several embodiments, the bile acid or salt thereof comprises, consists essentially of, or consists of cholate, deoxycholate (DC), chenodeoxycholate (CDC), or a combination of any of the foregoing. In several embodiments, the bile acid salt comprises, consists essentially of, or consists of sodium cholate (SC), sodium deoxycholate (SDC), sodium chenodeoxycholate (SCDC), or a combination of any of the foregoing.

In several embodiments, the bile salt comprises, consists essentially of, or consists of sodium taurocholate (STC). In several embodiments, the STC is present at a concentration of 1.0 mg/mL to 15.0 mg/mL. In several embodiments, the STC is present at a concentration of 8.0 mg/mL to 12.0 mg/mL. In several embodiments, the STC is present at a concentration of 9.0 mg/mL to 11.0 mg/mL. In several embodiments, the STC is present at a concentration of 10.0 mg/mL. In several embodiments, the STC is present in a dose amount of 0.1 mg to 1.5 mg. In several embodiments, the STC is present at a concentration of 5.0 mg/mL to 11.0 mg/mL. In several embodiments, the STC is present at a concentration of 7.0 mg/mL to 9.0 mg/mL.

In several embodiments, the pharmaceutical formulation is acidic (e.g., the pharmaceutical formulation is an aqueous solution having an acidic pH). In several embodiments, the pH of the pharmaceutical formulation ranges from 3.0 to 4.5. In several embodiments, the pH of the pharmaceutical formulation ranges from 3.7 to 3.9. In several embodiments, the pH of the pharmaceutical formulation ranges from 3.75 to 3.85. In several embodiments, the pH is about 3.8.

In several embodiments, the pharmaceutical formulation comprises an antioxidant. In several embodiments, the antioxidant comprises, consists essentially of, or consists of sodium metabisulfite.

In several embodiments, the pharmaceutical formulation comprises a preservative. In several embodiments, the preservative comprises, consists essentially of, or consists of chlorobutanol.

In several embodiments, the pharmaceutical formulation comprises a metal complexing agent. In several embodiments, the metal complexing agent comprises, consists essentially of, or consists of disodium edetate dihydrate (EDTA).

In several embodiments, the pharmaceutical formulation comprises a buffer. In several embodiments, the buffer comprises, consists essentially of, or consists of a citrate buffer (e.g., comprising, consisting of, or consisting essentially of citric acid and sodium citrate).

In several embodiments, the pharmaceutical formulation comprises a tonicity agent. In several embodiments, the tonicity agent comprises, consists essentially of, or consists of sodium chloride.

In several embodiments, the pharmaceutical formulation comprises a pH adjustor and/or a pH adjustor is added to the formulation to adjust the pH of the formulation. In several embodiments, the pH adjustor comprises, consists essentially of, or consists of hydrochloric acid, sodium hydroxide, or a combination thereof.

In several embodiments, the pharmaceutical formulation is configured to be delivered at a dose volume of about 0.05 mL to about 0.25 mL. In several embodiments, the pharmaceutical formulation is configured to be delivered at a dose volume of about 0.10 mL.

In several embodiments, if the bile acid, or the salt thereof, causes decreased cilia in a respiratory epithelium of a subject (e.g., a human subject), then such decreased cilia is substantially reversed and/or resolved within 7 days. In several embodiments, if the bile acid, or the salt thereof, causes hyperplasia of a respiratory epithelium of a subject (e.g., a human subject), then such hyperplasia is substantially reversed and/or resolved within 7 days.

Several embodiments pertain to a pharmaceutical formulation configured for IN delivery. In several embodiments, the pharmaceutical formulation comprises, consists essentially of, or consists of 1.0 mg/mL to 25.0 mg/mL of epinephrine (or a pharmaceutically acceptable salt thereof), 1.0 mg/mL to 15.0 mg/mL of a bile acid (or a salt thereof), a buffer, 3.0 mg/mL to 8.0 mg/mL of a preservative, 1.0 mg/mL to 4.0 mg/mL of a tonicity agent, 0.01 mg/mL to 0.05 mg/mL of metal complexing agent, and 0.1 mg/mL to 1.0 mg/mL of an antioxidant. In several embodiments, the pharmaceutical formulation has a pH of 2.2 to 5.0. In several embodiments, the pharmaceutical formulation is configured for IN delivery. In several embodiments, the buffer comprises an acid and its conjugate base. In several embodiments, the acid (e.g., citric acid, a hydrate of citric acid, etc.) is present in an amount ranging from 1.0 mg/mL to 8.0 mg/mL and the conjugate base (e.g., sodium citrate, a hydrate of or dihydrate of sodium citrate, etc.) is present in an amount ranging from 5.0 mg/mL to 10.0 mg/mL.

Several embodiments pertain to a pharmaceutical formulation configured for IN delivery. In several embodiments, the pharmaceutical formulation comprises, consists essentially of, or consists of 1.0 mg/mL to 25.0 mg/mL of epinephrine (or a pharmaceutically acceptable salt thereof), 1.0 mg/mL to 15.0 mg/mL of sodium taurocholate (STC), 1.0 mg/mL to 8.0 mg/mL of citric acid (e.g., citric acid, citric acid monohydrate, etc.), a buffer, 1.0 mg/mL to 4.0 mg/mL of sodium chloride, 0.01 mg/mL to 0.05 mg/mL of disodium edetate dihydrate, and 0.1 mg/mL to 1.0 mg/mL of sodium metabisulfite. In several embodiments, the pharmaceutical formulation has a pH of 2.2 to 5.0. In several embodiments, the pharmaceutical formulation is configured for IN delivery. In several embodiments, the buffer comprises, consists essentially of, or consists of 5.0 mg/mL to 10.0 mg/mL of sodium citrate (e.g., sodium citrate, sodium citrate dehydrate, etc.), 3.0 mg/mL to 8.0 mg/mL of chlorobutanol (e.g., chlorobutanol, chlorobutanol hemihydrate, etc.).

Several embodiments pertain to a pharmaceutical formulation configured for IN delivery. In several embodiments, the pharmaceutical formulation comprises, consists essentially of, or consists of 5.0 mg/mL to 13.0 mg/mL of epinephrine (or a pharmaceutically acceptable salt thereof), 5.0 mg/mL to 12.0 mg/mL of a bile acid, or a salt thereof, a buffer, 3.0 mg/mL to 8.0 mg/mL of a preservative, 1.0 mg/mL to 4.0 mg/mL of a tonicity agent, 0.01 mg/mL to 0.05 mg/mL of metal complexing agent, and 0.1 mg/mL to 1.0 mg/mL of an antioxidant. In several embodiments, the pharmaceutical formulation has a pH of 2.2 to 5.0. In several embodiments, the pharmaceutical formulation is configured for IN delivery. In several embodiments, the buffer comprises an acid and its conjugate base. In several embodiments, the acid (e.g., citric acid, a hydrate of citric acid, etc.) is present in an amount ranging from 1.0 mg/mL to 8.0 mg/mL and the conjugate base (e.g., sodium citrate, a hydrate of sodium citrate, etc.) is present in an amount ranging from 5.0 mg/mL to 10.0 mg/mL.

Several embodiments pertain to a pharmaceutical formulation configured for IN delivery. In several embodiments, the pharmaceutical formulation comprises, consists essentially of, or consists of 5.0 mg/mL to 13.0 mg/mL of epinephrine (or a pharmaceutically acceptable salt thereof), 5.0 mg/mL to 12.0 mg/mL of a bile salt (e.g., STC), 3.0 mg/mL to 5.0 mg/mL of citric acid (e.g., citric acid, citric acid monohydrate, etc.), 6.0 mg/mL to 10.0 mg/mL of sodium citrate (e.g., sodium citrate, sodium citrate dehydrate, etc.), 4.0 mg/mL to 7.0 mg/mL of chlorobutanol (e.g., chlorobutanol, chlorobutanol hemihydrate, etc.), 1.0 mg/mL to 5.0 mg/mL of sodium chloride, 0.01 mg/mL to 0.05 mg/mL of disodium edetate dihydrate, and 0.1 mg/mL to 1.0 mg/mL of sodium metabisulfite. In several embodiments, the pharmaceutical formulation has a pH of 3.7 to 3.9. In several embodiments, the pharmaceutical formulation is configured for IN delivery.

Several embodiments pertain to a pharmaceutical formulation configured for IN delivery. In several embodiments, the pharmaceutical formulation comprises, consists essentially of, or consists of 7.0 mg/mL to 9.0 mg/mL of epinephrine (or a pharmaceutically acceptable salt thereof), 7.0 mg/mL to 9.0 mg/mL of STC, a buffer, 4.0 mg/mL to 7.0 mg/mL of chlorobutanol (e.g., chlorobutanol, chlorobutanol hemihydrate, etc.), 1.0 mg/mL to 5.0 mg/mL of sodium chloride, 0.01 mg/mL to 0.05 mg/mL of disodium edetate dihydrate, and 0.1 mg/mL to 1.0 mg/mL of sodium metabisulfite. In several embodiments, the pharmaceutical formulation has a pH of 3.7 to 3.9. In several embodiments, the pharmaceutical formulation is configured for IN delivery. In several embodiments, the buffer comprises an acid and its conjugate base. In several embodiments, the acid (e.g., citric acid, a hydrate of citric acid) is present in an amount ranging from 3.0 mg/mL to 5.0 mg/mL and the conjugate base (e.g., sodium citrate, a hydrate of sodium citrate) is present in an amount ranging from 6.0 mg/mL to 10.0 mg/mL.

Also disclosed are methods of providing a rapid delivery of epinephrine to a patient by IN delivery using the disclosed epinephrine formulations for various treatments, or indications.

Some embodiments provide a method for rapid delivery of epinephrine to a human patient. In several embodiments, the method treats a condition of the patient. In several embodiments, the rapid delivery is via the intranasal route. In several embodiments, the method comprises administering a dose amount of epinephrine, or a pharmaceutically acceptable salt thereof, from a pharmaceutical formulation as disclosed above or elsewhere herein to at least one nostril of a human patient. In several embodiments, the method comprises administering the epinephrine intranasally. In several embodiments, the method comprises administering the epinephrine by intranasal (IN) delivery using a nasal spray. In several embodiments, the formulation is configured to provide and/or achieves an epinephrine Cₘₐₓ of 5 ng/mL to 15 ng/mL and a tₘₐₓ of less than 15 minutes.

In several embodiments, the epinephrine, or the pharmaceutically acceptable salt thereof, is provided in the formulation at a concentration in the range of 10 mg/mL to 14 mg/mL. In several embodiments, the epinephrine, or the pharmaceutically acceptable salt thereof, is provided in the formulation at a concentration of 12 mg/mL.

In several embodiments, the epinephrine, or the pharmaceutically acceptable salt thereof, is provided in the formulation at a range of 0.1 mg to 2.5 mg. In several embodiments, the epinephrine, or the pharmaceutically acceptable salt thereof, is provided in the formulation at a range of 0.5 mg to 1.3 mg.

In several embodiments, the formulation is configured to provide and/or achieves a tₘₐₓ of equal to or less than 12 minutes. In several embodiments, the formulation is configured to provide and/or achieves a tₘₐₓ of equal to or less than 10 minutes. In several embodiments, the formulation is configured to provide and/or achieves a tₘₐₓ of equal to or less than 5 minutes. In several embodiments, the formulation is configured to provide and/or achieves a tₘₐₓ of equal to or less than 3 minutes. In several embodiments, the formulation is configured to provide and/or achieves a Cₘₐₓ in a range of 10 ng/mL to 15 ng/mL. In several embodiments, the formulation is configured to provide and/or achieves a AUC_{0-10 min} in a range of 50 (ng*min)/mL to 80 (ng*min)/mL. In several embodiments, the formulation is configured to provide and/or achieves a AUC₀₋₃₀ₘᵢₙ in a range of 100 (ng*min)/mL to 170 (ng*min)/mL. In several embodiments, the formulation is configured to provide and/or achieves a AUC₀₋₁₈₀ₘᵢₙ in a range of 150 (ng*min)/mL to 300 (ng*min)/mL.

In several embodiments, a single spray of the nasal spray administers a dose volume of 0.10 mL to 0.25 mL of the pharmaceutical formulation. In several embodiments, the single spray of the nasal spray discharges a dose volume of about 0.10 mL of the pharmaceutical formulation.

In several embodiments, the condition is an allergic reaction. In several embodiments, the condition is anaphylaxis. In several embodiments, the condition is hypotension associated with septic shock, and the pharmaceutical formulation is used to increase mean arterial blood pressure in patients with hypotension associated with septic shock.

### BRIEF DESCRIPTION OF THE DRAWINGS

Exemplary features and advantages of certain exemplary embodiments of the present disclosure will become more apparent from the following description of certain exemplary embodiments thereof when taken in conjunction with the accompanying drawings.

FIG. 1 is a graph illustrating the Bile Salt (e.g., STC) Enhancement Effect on IN formulations based on Bile Salt (e.g., STC) Enhancement Factor versus Bile Salt (e.g., STC) concentration utilizing sodium taurocholate (STC) as the bile salt, which is further detailed in Example 1.

FIG. 2 is a graph illustrating the local irritation of the nasal mucosa by Bile Salt (e.g., STC) and in particular, the average total irritation points (TIP) for the findings in Example 3 versus time after IN treatment.

FIG. 3 is a graph illustrating the local irritation of the nasal mucosa by Bile Salt (e.g., STC) and in particular, the average M3,4 for the findings in Example 3 versus time after IN treatment.

FIG. 4A is a graph illustrating the local irritation of the nasal mucosa by Bile Salt (e.g., STC) and in particular, the TIP for erosion/flattening in Example 3 versus time after IN treatment.

FIG. 4B is a graph illustrating the local irritation of the nasal mucosa by Bile Salt (e.g., STC) and in particular, the TIP for decreased cilia in Example 3 versus time after IN treatment.

FIG. 4C is a graph illustrating the local irritation of the nasal mucosa by Bile Salt (e.g., STC) and in particular, the TIP for hyperplasia in Example 3 versus time after IN treatment.

FIG. 5A is a graph illustrating the relative bioavailability of epinephrine, IN versus IM, using two exemplary bile salts STC and sodium taurochenodeoxycholate (STCDC), which is further detailed in Example 4.

FIG. 5B is a graph illustrating the mean epinephrine concentration in rat serum from 0 min to 180 minutes utilizing STC as the bile salt, which is further detailed in Example 4.

FIG. 5C is a graph illustrating the mean epinephrine concentration in rat serum from 0 min to 180 minutes utilizing STCDC as the bile salt, which is further detailed in Example 4.

FIG. 6A is a graph illustrating the average total observation point (TOP) of a histopathologic study of a rat's nasal mucosa, using STCDC as the exemplary bile salt as further detailed in Example 5.

FIG. 6B is a graph illustrating the average occurrence of Level 3 (Moderate) of a histopathologic study of a rat's nasal mucosa, using STCDC as the exemplary bile salt as further detailed in Example 5.

FIG. 6C is a graph illustrating the average occurrence of Level 4 (Marked) of a histopathologic study of a rat's nasal mucosa, using STCDC as the exemplary bile salt as further detailed in Example 5.

FIG. 6D is a graph illustrating the average TOP of a histopathologic study of a rat's nasal mucosa, using STCDC as the exemplary bile salt as further detailed in Example 5.

FIG. 7A is a graph illustrating the average occurrence levels (AOL) of a histopathologic study of a rat's nasal mucosa for Group 3 in Example 5, using STCDC as the exemplary bile salt.

FIG. 7B is a graph illustrating the AOL of a histopathologic study of a rat's nasal mucosa for Group 4 in Example 5, using STCDC as the exemplary bile salt.

FIG. 7C is a graph illustrating the AOL of a histopathologic study of a rat's nasal mucosa for Group 5 in Example 5, using STCDC as the exemplary bile salt.

FIG. 7D is a graph illustrating the AOL of a histopathologic study of a rat's nasal mucosa for Group 6 in Example 5, using STCDC as the exemplary bile salt.

FIG. 8A is a graph illustrating the Cₘₐₓ for various embodiments of IN pharmaceutical formulations as disclosed herein versus the Cₘₐₓ for an IM comparator formulation in human subjects.

FIG. 8B is a graph illustrating the AUC_{0-t*} for various embodiments of IN pharmaceutical formulations as disclosed herein versus the AUC_{0-t*} for an IM comparator formulation in human subjects.

FIG. 8C is a graph illustrating the AUC_{0-10 min} for various embodiments of IN pharmaceutical formulations as disclosed herein versus the AUC_{0-10 min} for an IM comparator formulation in human subjects.

FIG. 8D is a graph illustrating the AUC₀₋₃₀ₘᵢₙ for various embodiments of IN pharmaceutical formulations as disclosed herein versus the AUC₀₋₃₀ₘᵢₙ for an IM comparator formulation.

FIG. 8E is a graph illustrating the AUC_{0-6 hrs} for various embodiments of IN pharmaceutical formulations as disclosed herein versus the AUC_{0-6 hrs} for an IM comparator formulation in human subjects.

FIG. 8F is a graph illustrating the AUC₀₋₂₈ for various embodiments of IN pharmaceutical formulations as disclosed herein versus the AUC₀₋₂₈ for an IM comparator formulation in human subjects.

FIG. 8G is a table providing a summary of baseline corrected epinephrine PK parameters per protocol population.

FIG. 9 is a blood concentration versus time curve for epinephrine using embodiments as disclosed herein and an IM comparator formulation.

FIG. 10 is a blood concentration versus time curve for STC using embodiments as disclosed herein.

FIG. 11A-11H provide pharmacodynamic data for various embodiments of IN pharmaceutical formulations as disclosed herein versus the an IM comparator formulation.

### DETAILED DESCRIPTION

Several embodiments herein pertain to intranasal compositions (e.g., pharmaceutical formulations) comprising epinephrine and a bile acid, and/or methods for making or using such compositions. In several embodiments, the composition is useful in intranasal delivery of epinephrine. In several embodiments, the bile acid enhances the absorption of epinephrine through the nasal mucosa of the nasal cavity. In several embodiments, the bile acid is provided as a bile acid salt. In several embodiments, the composition may further comprise one or more additional pharmaceutically acceptable carriers and/or one or more additional pharmaceutically acceptable excipients. The matters exemplified in this description are provided to assist in a comprehensive understanding of exemplary embodiments of the invention with reference to the accompanying drawings. While the present disclosure has been described in connection with certain embodiments, it is to be understood that the invention is not limited to the disclosed embodiments, but, on the contrary, is intended to cover various modifications and arrangements included within the spirit and scope of the appended claims, and equivalents thereof. Accordingly, those of ordinary skill in the art will recognize that various changes and modifications of the exemplary embodiments described herein can be made without departing from the scope and spirit of the claimed invention. No single component or collection of components is essential or indispensable. Any feature, structure, component, material, step, or method that is described and/or illustrated in any embodiment in this specification can be used with or instead of any feature, structure, component, material, step, or method that is described and/or illustrated in any other embodiment in this specification.

The word “exemplary” is used herein to mean “serving as an example, instance, or illustration.” Any embodiment described herein as “exemplary” is not necessarily to be construed as preferred or advantageous over other embodiments. Likewise, the term “embodiments” does not require that all embodiments include the discussed feature, advantage or mode of operation.

Unless otherwise defined herein, scientific and technical terms used in connection with embodiments of present disclosure shall have the meanings that are commonly understood by those of ordinary skill in the art. Nomenclatures used in connection with, and techniques described herein are those known and commonly used in the art. Also, descriptions of well-known functions and constructions are omitted for clarity and concision.

“Pharmaceutical formulation” refers to a formulation comprising at least one active pharmaceutical ingredient (API). For brevity herein, “IN pharmaceutical formulations” refers to pharmaceutical formulations configured for IN delivery. “IN epinephrine pharmaceutical formulations” refers to IN pharmaceutical formulations including at least epinephrine, or a pharmaceutically acceptable salt thereof, for IN delivery.

“Pharmaceutically acceptable” refers to an ingredient in the pharmaceutical formulation that is compatible with the other ingredients in the formulation, and does not cause excess harm to the patient receiving the pharmaceutical formulation.

The term “pharmaceutically acceptable salt” refers to salts that retain the biological effectiveness and properties of a compound, which are not biologically or otherwise undesirable for use in a pharmaceutical. In many cases, the compounds herein (including bile acids) are capable of forming acid and/or base salts by virtue of, for example, the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts (e.g., which form salts with bile acids) can be formed with inorganic and organic bases. Inorganic bases from which salts (e.g., bile salts) can be derived include, for example, sodium, potassium, lithium, ammonium, calcium, magnesium, iron, zinc, copper, manganese, aluminum, and the like; particularly ammonium, potassium, sodium, calcium and magnesium salts. Organic bases from which salts (e.g., bile salts) can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in U.S. Pat. No. 4,783,443A (incorporated by reference herein in its entirety).

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety. A carrier may be aqueous or may be water or saline (e.g., water, saline, saline for injection).

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are relieved, lessened, reduced, diminished, treated, prevented, remedied, healed, stabilized, alleviated, altered, ameliorated, or eliminated.

The terms “treatment,” “treating,” “treat” and the like shall be given its ordinary meaning and shall also include herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein shall be given its ordinary meaning and shall also cover any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; and/or (c) relieving the disease symptom, e.g., causing regression of the disease or symptom.

The “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, horses, cats, dog, rabbits, rodents (e.g., rats or mice), and monkeys. Human subjects include neonates, infants, juveniles, adults and geriatric subjects.

As used herein, the term “Cₘₐₓ” is given its plain and ordinary meaning and refers to the maximum (or peak) plasma concentration of an agent after it is administered. A Cₘₐₓ may be reported as the geometric and/or arithmetic mean of individual Cₘₐₓ values from a given patient population.

As used herein, the term “tₘₐₓ” is given its plain and ordinary meaning and refers to the length of time required for an agent to reach maximum plasma concentration after the agent is administered. A tₘₐₓ may be reported as the geometric and/or arithmetic mean of individual tₘₐₓ values from a given patient population.

As used herein, the term “AUC” is given its plain and ordinary meaning and refers to the calculated area under the curve, referring to a plasma concentration-time curve (e.g., the definite integral in a plot of drug concentration in blood plasma vs. time). AUC may be reported as the geometric and/or arithmetic mean of individual AUC values from a given patient population. AUC may be reported as a partial AUC within a given time frame. For example, for the AUC between time points “a” and “b”, the AUC within that time window is reported as AUC_{a-b}. To illustrate, the AUC from time 0 (when the API is administered) to a time point 10 minutes later, to a time point 30 minutes later, to a time point 180 minutes later, to a time point 6 hours later, or a time where the blood concentration is less than the limit of detection, AUC is reported as AUC_{0-10 min}, AUC₀₋₃₀ₘᵢₙ, AUC_{0-180 min}, AUC_{0-6 hr}, and AUC_{0-∞}, respectively. Other start points for AUC values may be taken by subtracting one AUC from another. For instance, an AUC_{30 min-6hr}, may be calculated by subtracting AUC₀₋₃₀ₘᵢₙ from AUC₀₋₆ₕᵣ. Other AUC values may be similarly calculated (e.g., AUC_{10 min-30 min}, AUC_{10 min-180 min}, AUC_{30 min-180 min}, AUC_{10 min-6hr}, AUC_{180 min-180 min}, AUC_{10 min-∞}, AUC_{30 min-∞}, AUC_{180 min-∞}, and AUC_{6 hr-∞}).

When referring to the amount present for one or more ingredients, the terms “or ranges including and/or spanning the aforementioned values” (and variations thereof) is meant to include any range that includes or spans the aforementioned values. For example, when the concentration of an ingredient is expressed as 1 mg/mL, 5 mg/mL, 10 mg/mL, 20 mg/mL, “or ranges including and/or spanning the aforementioned values,” this includes ranges for the ingredient spanning from 1 mg/mL to 20 mg/mL, 1 mg/mL to 10 mg/mL, 1 mg/mL to 5 mg/mL, 5 mg/mL to 20 mg/mL, 5 mg/mL to 10 mg/mL, and 10 mg/mL to 20 mg/mL.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and immaterial deviations are within the scope of the present teachings herein. In this application, the use of the singular includes the plural unless specifically stated otherwise.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction “or” should not be read as requiring mutual exclusivity among that group, but rather should be read as “and/or” unless expressly stated otherwise.

Additionally, the phrase “consisting essentially of” will be understood to include those elements specifically recited and those additional elements that do not materially affect the basic and novel characteristics of the claimed technology. The phrase “consisting of” excludes any element not specified.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used together.

### Introduction

Currently, pre-filled syringes of epinephrine injection (e.g., auto-injector) given intramuscularly (IM) or subcutaneously, by needle, are the only approved first-aid treatment of anaphylaxis. Auto-injectors have several limitations and disadvantages in its clinical use. For instance, the standard 15.2 mm needle length was found to be inadequate for IM delivery in 19% of at-risk female patients based on sonographic measurement of skin-to-muscle depth at mid-anterolateral thigh. Several use-related injuries have also been reported including unintentional injection, and lacerations (e.g., thigh lacerations) in healthcare providers and children. On a practical level, patients may be reluctant to self-inject because of a general (or extreme) fear of needles, bleeding, pain/discomfort of a needle puncture, bruising, fear of needing multiple attempts to properly self-inject, anxiety, and inability to self-inject properly in emergencies when the patient may not be calm or composed. Repeated training may be needed for a patient to learn how to properly self-inject.

Epinephrine is typically administered in emergencies and not for routine (e.g., daily) use, so patients may forget the proper techniques due to the lack of routine use. And especially in emergency situations, patients may not be in a calm or composed state of mind to properly self-inject the epinephrine drug. For example, proper IM self-injection requires the patient to know and remember the optimal sites on the human body (e.g., thigh muscle) to inject in order for the drug to be absorbed relatively quickly into the bloodstream. Otherwise, if the patient injects the IM drug at non-optimal sites of the human body, then it may take a longer period of time for the drug to be absorbed into the bloodstream, which may be counter-productive in an emergency treatment. In addition, improper self-injection may lead to bleeding, bruising, swelling, numbness, tingling, lacerations, or other pain/discomfort.

A potential alternative to injection delivery is to deliver the drug through the intranasal route of administration, also referred to as intranasal (IN) delivery herein. While some attempts to prepare compositions for intranasal administration have been attempted, these suffer from side effects, including pain in the nasal passages or other symptoms. Moreover, even where patients tolerate IN route, the compositions used often have pharmacokinetic (PK) profiles that are unlike those for the IM route and/or are disadvantageous. For instance, to achieve a desired Cₘₐₓ or tₘₐₓ, such as that provided by IM administration of the drug, the required dose of epinephrine in an IN formulation may expose the patient to an unnecessarily high amount of epinephrine overall (e.g., the AUC, such as the AUC_{0-∞}, may be unacceptably high or higher than needed). Where a desired AUC value is achieved (such an AUC comparable to that provided by IM administration), the Cₘₐₓ of epinephrine may be too low or the tₘₐₓ to high, or vice versa. Given the balance of requirements needed for effective administration of epinephrine, there is an unmet medical need to develop epinephrine drug products to overcome these disadvantages. Several embodiments disclosed herein solve one or more of these problems or others by providing pharmaceutical formulations suitable for IN delivery that are well tolerated and have a desirable PK profiles. In several embodiments, using the IN formulations disclosed herein, surprisingly, desirable PK parameters, pharmacodynamic, safety, and/or tolerability profiles can be achieved.

For example, using bile acids (including bile acid salts as disclosed herein) as absorption enhancers, it has now been found that the intranasal (IN) route of administration can provide a rapid onset of drug action. In particular, using bile acids (and the salts thereof) the nasal cavity provides direct access to the bloodstream, thereby avoiding first-pass metabolism of portal circulation, and leading to a rapid onset of drug action. This is especially advantageous because the IN route is a non-invasive drug delivery method. By contrast, IM delivery requires the patient to inject deep into the muscle at the optimal sites, otherwise the drug may not be readily absorbed into the bloodstream. In addition, the IN compositions disclosed herein offer several advantages over IM, such as being easy to use, painless, easy to carry, and self-administrable without use of needles. Surprisingly, the IN compositions disclosed herein, which comprise bile acids (or salts thereof) as enhancing agents, achieve similar PK profiles to the IM route or even potentially improved profiles.

As disclosed elsewhere herein, IN delivery utilizes drug absorption through the nasal cavity and more particularly, the nasal mucosa (also known as the respiratory mucosa), which is a highly vascularized mucous membrane that lines the nasal cavity. The nasal mucosa is made up of primarily two layers, an upper epithelial layer that is predominantly lipophilic, and a sublayer known as the lamina propria. The upper epithelial layer is generally made up of epithelium, cilia, mucus (mucin), goblet cells (mucus-producing cells), and others cells. Notably, the lamina propria is highly vascularized with an extensive network of blood vessels, which can enable a drug to be rapidly absorbed into the bloodstream. However, in order to reach these blood vessels in the lamina propria, a pharmacological challenge is to develop pharmaceutical formulations that enhance drug absorption through the predominantly lipophilic upper epithelial layer.

The nasal cavity is the main passageway for air into and out of the lungs. Thus, a primary function of the nasal mucosa is to serve as an immune defense against foreign agents, such as drugs, allergens, pathogens, viruses, bacteria, dirt particles, and other airborne particulates. Consequently, it is a pharmacological challenge to achieve epinephrine absorption through the nasal mucosa. Additionally, epinephrine, by itself, has low membrane permeability. Thus, when aqueous epinephrine is delivered by the IN route, the absorption is very low. The bioavailability (BA), based on the area under curve (AUC) in the plasma concentration of epinephrine, is only approximately 5% relative to that for the same dose of epinephrine delivered by the IM route. For these reasons, epinephrine has historically been deemed to have limited therapeutic use by IN delivery. Therefore, an absorption enhancer for IN delivery strong enough to enhance the absorption of the epinephrine to a reach a BA similar to that achieved by IM route of EpiPen® (1 mg/mL) is needed.

Another challenge is enhancing the absorption of epinephrine in the network of blood vessels in the nasal mucosa without causing (e.g., minimizing) toxicity or damage to the nasal cavity. Minimizing local toxicity to the nasal cavity is important because the nasal mucosa provides a number of critical functions for the body, such as humidifying inhaled air, serving as an immune defense against foreign agents. Because of its role in the body, the nasal mucosa is also one of the most commonly infected tissues. Inflammation of the nasal mucosa may cause a stuffy nose, headaches, mouth breathing, and other symptoms. This inflammation can be exacerbated by IN delivery of pharmaceutical agents, making it more challenging to develop drug formulations for IN administration.

Historically, bile acids/salts have not been successfully implemented for clinical use due to various toxicity issues. In particular, bile acids/salts have limited clinical use because of suspected irreversible damage to the mucosa and ciliotoxicity. In addition, prior to the compositions disclosed herein, bile acids/salts caused nasal irritation when used above a certain concentration, such as a concentration above 3 mg/mL. Sodium taurocholate (STC) is an example of such a bile salt. Surprisingly, the IN compositions disclosed herein are well-tolerated by patients. To date, the U.S. Food and Drug Administration (FDA) database has not listed STC as an inactive ingredient for any approved drugs or drug formulations. Several bile acids, including taurocholic acid, are otherwise included as safe in FDA's GRAS Substances (SCOGS) database.

IN pharmaceutical formulations should deliver epinephrine into the bloodstream while also minimizing or reducing the absorption enhancer's toxicity. Embodiments of the present disclosure solve one or more of these pharmacological challenges (or others) by introducing IN pharmaceutical formulations including epinephrine, or a pharmaceutically acceptable salt thereof, and a bile acid, or a salt thereof, as the absorption enhancer. Also disclosed are methods of providing a rapid delivery of epinephrine to a patient by IN delivery using the disclosed epinephrine formulations for various treatments, or indications.

Exemplary embodiments of the present disclosure address one or more or all of the above problems and/or disadvantages (or others) by providing formulations and methods for the delivery of epinephrine. Additional objects, advantages, and salient features of exemplary embodiments of the present disclosure will become apparent to those skilled in the art from the following detailed description, which, taken in conjunction with the annexed drawings, discloses exemplary embodiments of the present disclosure.

### Intranasal Epinephrine Pharmaceutical Formulations

Disclosed herein are formulations (e.g., pharmaceutical formulations) configured for intranasal delivery. In several embodiment, the formulation comprises epinephrine, or a pharmaceutically acceptable salt thereof, and a bile acid (e.g., a bile acid or a salt thereof) as an absorption enhancer. In several embodiments, the absorption enhancer increases the absorption of epinephrine, the active pharmaceutical ingredient (API), after administration via the nasal passages. In several embodiments, the bile acid, or the salt thereof, serves as the absorption enhancer for enhancing the absorption of epinephrine into the bloodstream by intranasal delivery, as described herein. In several embodiments, the bile acid, or the salt thereof, is configured to enhance the absorption of epinephrine into the bloodstream by intranasal delivery, as described herein. In several embodiments, the formulation comprises one or more carriers (e.g., pharmaceutically acceptable carriers) and/or excipients (e.g., pharmaceutically acceptable excipients) as disclosed elsewhere herein. In several embodiments, the pharmaceutical formulations for intranasal (IN) delivery are configured for use in a human subject. In several embodiments of the IN epinephrine pharmaceutical formulations, the formulations include water (and/or are aqueous).

### Epinephrine and Pharmaceutically Acceptable Salts Thereof

In several embodiments, as described elsewhere herein, the composition comprises epinephrine as a free base or as a pharmaceutically acceptable salt. In several embodiments, the pharmaceutically acceptable salt of epinephrine is an acetate salt, a bitartrate salt, a carbonate salt, a citrate salt, a hydrochloride salt, a hydrocyanide salt, a hydrofluoride salt, a nitrate salt, a nitrite salt, a phosphate salt, a sulfate salt, or a combination of any one or more of the foregoing. In several embodiments, the present disclosure is not limited to these salt forms.

In several embodiments of the IN epinephrine pharmaceutical formulation, the epinephrine, or the pharmaceutically acceptable salt thereof, is present at a concentration of equal to or less than about: 1 mg/mL, 2.5 mg/mL, 5 mg/mL, 7.5 mg/mL, 10 mg/mL, 11 mg/mL, 12 mg/mL, 13 mg/mL, 14 mg/mL, 15 mg/mL, 20 mg/mL, 25 mg/ml, or ranges including and/or spanning the aforementioned values. For example, in several embodiments, the IN epinephrine pharmaceutical formulation comprises epinephrine or a salt thereof at a concentration ranging from 7.5 mg/mL to 15 mg/mL, 10 mg/mL to 14 mg/mL, 5 mg/mL to 15 mg/mL, 10 mg/mL to 20 mg/mL, 20 mg/mL to 25 mg/mL etc.

In several embodiments of the IN epinephrine pharmaceutical formulations, the epinephrine, or the pharmaceutically acceptable salt thereof, is present at a concentration of 1.0 mg/mL to 25.0 mg/mL, or any concentration range subsumed therein, including but not limited to, 5.0 mg/mL to 15.0 mg/mL, 5.0 mg/mL to 13.0 mg/mL, 7.5 mg/mL to 12.5 mg/mL, 8.0 mg/mL to 12.0 mg/mL, 9.0 mg/mL to 11.0 mg/mL, 9.5 mg/mL to 10.5 mg/mL, or 7.0 mg/mL to 9.0 mg/mL. In several embodiments of the IN epinephrine pharmaceutical formulations, the epinephrine, or the pharmaceutically acceptable salt thereof, is present at a concentration of equal to or less than about: 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, 12.0 mg/mL, 12.5 mg/mL, 13.0 mg/mL, 13.5 mg/mL, 14.0 mg/mL, 14.5 mg/mL, 15.0 mg/mL, 15.5 mg/mL, 16.0 mg/mL, 16.5 mg/mL, 17.0 mg/mL, 17.5 mg/mL, 18.0 mg/mL, 18.5 mg/mL, 19.0 mg/mL, 19.5 mg/mL, 20.0 mg/mL, 20.5 mg/mL, 21.0 mg/mL, 21.5 mg/mL, 22.0 mg/mL, 22.5 mg/mL, 23.0 mg/mL, 23.5 mg/mL, 24.0 mg/mL, 24.5 mg/mL, 25.0 mg/mL, or ranges including and/or spanning the aforementioned values.

In several embodiments of the IN epinephrine pharmaceutical formulations, the epinephrine, or the pharmaceutically acceptable salt thereof, is present at a concentration of 5.0 mg/mL to 13.0 mg/mL. In several embodiments of the IN epinephrine pharmaceutical formulations, the epinephrine, or the pharmaceutically acceptable salt thereof, is present at a concentration of equal to or less than about: 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 11.5 mg/mL, 12.0 mg/mL, 12.5 mg/mL, 13.0 mg/mL, or ranges including and/or spanning the aforementioned values.

In several embodiments of the IN epinephrine pharmaceutical formulations, the epinephrine, or the pharmaceutically acceptable salt thereof, is present at a concentration of 6.0 mg/mL to 10.0 mg/mL. In several embodiments of the IN epinephrine pharmaceutical formulations, the epinephrine, or the pharmaceutically acceptable salt thereof, is present at a concentration of equal to or less than about: 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, or ranges including and/or spanning the aforementioned values.

In several embodiments, the IN epinephrine pharmaceutical formulation comprises epinephrine, or the pharmaceutically acceptable salt thereof, at a molarity of equal to or less than about: 0.005 M, 0.02 M, 0.04 M, 0.05 M, 0.06 M, 0.07 M, 0.08 M, 0.1 M, 0.15 M, or ranges including and/or spanning the aforementioned values. For example, in several embodiments, the IN epinephrine pharmaceutical formulation comprises epinephrine or a salt thereof at a molarity ranging from 0.04 M to 0.07 M, 0.05 M to 0.07 M, 0.02 M to 0.1 M, 0.05 M to 0.07 M, etc.

In several embodiments, as disclosed elsewhere herein, the formulation is provided in a IN dosing device. In several embodiments, the dosing device delivers a dose of the composition to a patient (e.g., a patient in need of treatment). In several embodiments, a dose of the pharmaceutical formulation comprises epinephrine, or a pharmaceutically acceptable salt thereof, in an amount ranging from 0.1 mg to 2.5 mg, or any amount range subsumed therein, including but not limited to, 0.1 mg to 2.5 mg, 0.1 mg to 2.25 mg, 0.1 mg to 2.0 mg, 0.1 mg to 1.75 mg, 0.1 mg to 1.5 mg, 0.1 mg to 1.25 mg, 0.1 mg to 1.0 mg, 0.1 mg to 0.75 mg, 0.1 mg to 0.5 mg, 0.1 mg to 0.25 mg, 0.25 mg to 2.5 mg, 0.25 mg to 2.25 mg, 0.25 mg to 2.0 mg, 0.25 mg to 1.75 mg, 0.25 mg to 1.5 mg, 0.25 mg to 1.25 mg, 0.25 mg to 1.0 mg, 0.25 mg to 0.75 mg, 0.25 mg to 0.5 mg, 00.5 mg to 2.5 mg, 0.5 mg to 2.25 mg, 0.5 mg to 2.0 mg, 0.5 mg to 1.75 mg, 0.5 mg to 1.5 mg, 0.5 mg to 1.3 mg, 0.5 mg to 1.25 mg, 0.5 mg to 1.0 mg, 0.5 mg to 0.75 mg, 0.75 mg to 2.5 mg, 0.75 mg to 2.25 mg, 0.75 mg to 2.0 mg, 0.75 mg to 1.75 mg, 0.75 mg to 1.5 mg, 0.75 mg to 1.3 mg, 0.75 mg to 1.25 mg, 0.75 mg to 1.0 mg, 1.0 mg to 2.5 mg, or ranges including and/or spanning any of the aforementioned values.

In several embodiments, a dose of the pharmaceutical formulation comprises epinephrine, or a pharmaceutically acceptable salt thereof, in an amount equal to or less than about: 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, or ranges including and/or spanning the aforementioned values.

In several embodiments, a dose of the pharmaceutical formulation comprises epinephrine, or a pharmaceutically acceptable salt thereof, in an amount ranging from 0.1 mg to 2.5 mg, or any amount range subsumed therein, including but not limited to, 0.5 mg to 1.5 mg, 0.5 mg to 1.3 mg, 0.7 mg to 0.9 mg, 0.75 mg to 1.25 mg, 0.8 mg to 1.2 mg, 0.9 mg to 1.1 mg, 0.95 mg to 1.05 mg. In several embodiments, a dose of the pharmaceutical formulation comprises epinephrine, or a pharmaceutically acceptable salt thereof, in an amount equal to or less than about: 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, or 2.5 mg, or ranges including and/or spanning the aforementioned values.

In several embodiments, a dose of the pharmaceutical formulation comprises epinephrine, or a pharmaceutically acceptable salt thereof, in an amount ranging from 0.5 mg to 1.30 mg. In several embodiments, a dose of the pharmaceutical formulation comprises epinephrine, or a pharmaceutically acceptable salt thereof, in an amount equal to or less than about: 0.50 mg, 0.55 mg, 0.60 mg, 0.70 mg, 0.75 mg, 0.80 mg, 0.85 mg, 0.90 mg, 0.95 mg, 1.00 mg, 1.05 mg, 1.10 mg, 1.15 mg, 1.20 mg, 1.25 mg, 1.30 mg, or ranges including and/or spanning the aforementioned values.

In several embodiments, a dose of the pharmaceutical formulation comprises epinephrine, or a pharmaceutically acceptable salt thereof, in an amount ranging from 0.6 mg to 1.0 mg. In several embodiments, a dose of the pharmaceutical formulation comprises epinephrine, or a pharmaceutically acceptable salt thereof, in an amount equal to or less than about: 0.60 mg, 0.70 mg, 0.75 mg, 0.80 mg, 0.85 mg, 0.90 mg, 0.95 mg, 1.00 mg, or ranges including and/or spanning the aforementioned values.

In several embodiments of the IN epinephrine pharmaceutical formulations, a single administration (e.g., a single spray) provides a full dose of the pharmaceutical formulation. In several embodiments, a dose can be provided in a plurality of administrations from dosing device. For example, multiple sprays (e.g., equal to or greater than 2, 3, 4, 5 sprays) in quick succession. In the context of IN delivery, “to be taken at one time” covers the discharge of the dose volume in: (1) a single spray, or (2) two or more sprays in a very short amount of time, usually less than one minute. Thus, the dose volume containing the dose amount of the epinephrine, or the pharmaceutically acceptable salt thereof, can be administered in one or more nasal sprays. In several embodiments, the dose volume is administered in one spray of the nasal spray. In several embodiments, the dose volume is administered in two or more sprays of the nasal spray (e.g., 2, 3, 4, 5, or more sprays). In several embodiments, the dose amount of the epinephrine, or the pharmaceutically acceptable salt thereof, is administered in a single spray. In several embodiments, the dose amount of the epinephrine, or the pharmaceutically acceptable salt thereof, is administered in two or more sprays (e.g., 2, 3, 4, or more sprays).

In several embodiments, the dose volume of the IN epinephrine pharmaceutical formulation is from 0.01 mL to 0.30 mL. In several embodiments, the dose volume of the IN epinephrine pharmaceutical formulation is from 0.05 mL to 0.15 mL. In several embodiments, the dose volume of the IN epinephrine pharmaceutical formulation is about 0.10 mL. In several embodiments of the IN epinephrine pharmaceutical formulation, the dose volume is about 0.10 mL, which can be administered in a single nasal spray. In several embodiments, a dose volume of the IN epinephrine pharmaceutical formulation equal to or less than about: 0.01 mL, 0.05 mL, 0.075 mL, 0.1 mL, 0.2 mL, 0.3 mL, or ranges including and/or spanning the aforementioned values.

### Bile Acid/Salt Enhances Absorption of Epinephrine By IN Delivery

In several embodiments, as disclosed elsewhere herein, the IN epinephrine pharmaceutical formulation comprises a bile acid, or the salt thereof (e.g., a salt of any bile acid disclosed herein). For brevity, unless the language referring to a “bile acid” specifically excludes bile acid salts, any time the term bile acid is used, bile acid salts are also envisioned (e.g., pharmaceutically acceptable salts of bile acids). In several embodiments, the bile acid and/or bile acid salt acts as, or is configured to act as, an absorption enhancer. The term “absorption enhancer,” as used herein in the context of IN delivery, refers to an agent (e.g., excipient) in the pharmaceutical formulation whose function is to improve the absorption of the API (e.g., epinephrine) into the bloodstream by enhancing permeation of the API through the nasal mucosa.

In several embodiments, bile acids, or the salts thereof, can enhance the IN absorption of drugs into the system circulation via the nasal mucosa. As disclosed elsewhere herein, the nasal mucosa has two layers: (1) the outer epithelial layer, which is predominately lipophilic, and (2) the inner sublayer, known as the lamina propria, which comprises blood vessels for access to the bloodstream. Without being bound to any theory, in several embodiments, the bile acid, or the salt thereof, enhances the IN absorption of epinephrine (e.g., the API) by enabling access to the blood vessels in the lamina propria of the nasal mucosa. After the API is absorbed into the bloodstream, the API can be distributed throughout the human body via the circulatory system.

Bile acids are ionic amphiphilic compounds with a steroid skeleton. As demonstrated elsewhere herein, it has been found that bile acids have number of physiologically beneficial properties. In several embodiments, bile acids (or salts thereof) achieve lipid transport by solubilization of insoluble drug molecules. In several embodiments, the bile acid is configured to transport of polar drugs through hydrophobic barriers. In several embodiments, the bile acid inhibits enzyme activity. In several embodiments, without being bound to a theory, the bile acid aids in opening tight junctions between epithelial cells. In several embodiments, within the human body, bile acids are amphipathic and act as steroidal bio-surfactants. Bile acids are often derived from cholesterol in the liver. For example, the synthesis of bile salts is the major route for elimination of cholesterol from the body.

While the present disclosure is not limited by any particular mechanism or theory, it is believed that based on the above-described properties, bile acids/salts can enhance IN absorption by forming micelles and/or reverse micelles to enable transcellular passage of the API through the predominantly lipophilic upper epithelial layer of the nasal mucosa and into the blood vessels located in the lamina propria sublayer. In several embodiments, one or more goals of the present disclosure (or others) are accomplished using a micelle or reverse micelle forming agent to enhance IN delivery. In several embodiments, the micelle or reverse micelle forming agent is used as an enhancing agent, as disclosed elsewhere herein. In several embodiments, the micelle or reverse micelle forming agent is a bile acid or bile acid salt. Without being bound to any particular theory, it is also believed that, in some embodiments, bile acids/salts inhibit the tight junctions between the epithelial cells to enable paracellular passage of the API through the predominantly lipophilic upper epithelial layer of the nasal mucosa and into the blood vessels located in the lamina propria sublayer. In this regard, bile acids/salts can disrupt the hemidesmosomes or by bind to calcium in the tight junctions. In several embodiments, one or more goals of the present disclosure (or others) are accomplished using a hemidesmosome disrupting agent to enhance IN delivery. In several embodiments, the hemidesmosome disrupting agent is used as an enhancing agent, as disclosed elsewhere herein. In several embodiments, the hemidesmosome disrupting agent is a bile acid or bile acid salt.

Additionally, in several embodiments, bile acids/salts can and/or are configured to enhance IN absorption of APIs by inhibiting, degrading, or reducing enzymes, such as mucosal membrane peptidases, in the predominantly lipophilic upper epithelial layer of the nasal mucosa. Without being bound to a theory, it is believed that bile acids/salts may enhance absorption of APIs by reducing the viscosity or elasticity of the predominantly lipophilic upper epithelial layer of the nasal mucosa. Therefore, bile acids/salts can enhance IN absorptions of APIs through these aforementioned means or a combination thereof. In several embodiments, the enhancing agent is an enzyme inhibiting agent. In several embodiments, the enhancing agent changes the viscosity and/or elasticity of the epithelial layer of the nasal mucosa. In several embodiments, the bile acid (or salt thereof) is used as an agent to inhibit enzymatic degradation of the API (e.g., epinephrine) and/or to change the viscosity and/or elasticity of the epithelial layer of the nasal mucosa (e.g., to enhance delivery of epinephrine).

Bile salts can be formed when the conjugated bile acid complexes with sodium or other appropriate cations. As disclosed elsewhere herein, other suitable elements (e.g., ions of elements and/or cations that form salts), such as potassium, may also be used to complex with the conjugate bile acid to form bile salts. Bile acids/salts can be conjugated with an amino acid, such as glycine or taurine, to form conjugated bile acids/salts. Bile acids/salts are ionic amphiphilic compounds with a steroid skeleton.

The structure below is a common chemical structure of a bile acid. As shown, this common structure of a bile acid consists of four rings, three six carbon rings (A, B and C), and one five carbon ring (D). The structure below is a non-limiting representative chemical structure of an embodiment of a bile acid:

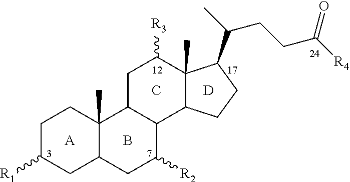

Wherein the numbering conforms to the steroid numbering system.

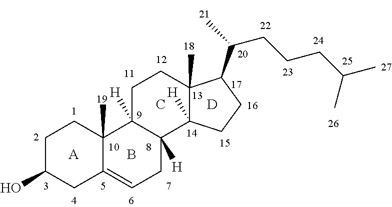

wherein the numbering conforms to the steroid numbering system.

Epinephrine, by itself, has low membrane permeability because it is hydrophilic and the upper epithelial layer of the nasal mucosa is predominantly lipophilic. For example, when aqueous epinephrine is delivered by the IN route, the absorption is very low. The bioavailability (BA), based on the area under curve (AUC) in the plasma concentration of epinephrine, is only approximately 5% relative to that for the same dose of epinephrine delivered by the IM route. In several embodiments, an absorption enhancer, such as a bile acid/salt, is needed to enhance the absorption of the epinephrine through the lipophilic portions of the nasal mucosa and into the bloodstream. For example, the disclosed epinephrine formulations having STC as the bile salt is able to increase the bioavailability of epinephrine by IN about 15 to 20 times compared to IN epinephrine without STC.

Bile acids/salts can be categorized into three main groups based on their conjugation with amino acids and their degree of hydroxylation. These three main groups are: (1) trihydroxy conjugates, (2) dihydroxy conjugates, and (3) unconjugated forms. In several embodiments, the bile acid and/or salt thereof of the IN pharmaceutical composition comprises a trihydroxy conjugate, a dihydroxy conjugate, an unconjugated form, or combinations of any of the foregoing. In several embodiments, combinations of bile acids and/or salts thereof may be used in the IN pharmaceutical formulation. In several embodiments, a plurality of different bile acids and/or salts thereof (e.g., 2, 3, 4, or more) may be used in the IN pharmaceutical formulation.

In several embodiments, the enhancing agent (e.g., absorption enhancer) in the IN pharmaceutical formulation is a trihydroxy conjugate (or a salt thereof). Exemplary embodiments of trihydroxy conjugates of bile acids that may be used in the intranasal formulations disclosed herein include, but are not limited to, glycocholate (e.g., glycocholic acid) (GC), taurocholate (e.g., taurocholic acid) (TC), glycohyocholate (e.g., glycohyocholic acid) (GHC), taurohyocholate (e.g., taurohyocholic acid) (THC), tauro-α-hyocholate (e.g., tauro-α-hyocholic acid) (T-α-MC), tauro-β-hyocholate (e.g., tauro-β-hyocholic acid) (T-β-MC), or a combination thereof. In several embodiments, the bile acid is taurocholic acid.

Exemplary embodiments of trihydroxy conjugates of bile salts that may be used in the intranasal formulations disclosed herein include, but are not limited to, sodium glycocholate (SGC), sodium taurocholate (STC), sodium glycohyocholate (SGHC), sodium taurohyocholate (STHC), sodium tauro-α-hyocholate (S-T-α-MC), sodium tauro-β-hyocholate (S-T-β-MC), or a combination thereof. Exemplary embodiments of trihydroxy conjugates of bile salts that may be used in the intranasal formulations disclosed herein include, but are not limited to, sodium glycolithocholate (SGLC), sodium glyco-7-oxo-lithocholate (SG-7-oxo-LC), sodium glycol-3α-6-keto-5β-cholate (S-glycol3α6keto-5β-cholate), or a combination thereof.

Other suitable forms of salts that may be used in the intranasal formulations disclosed herein are possible, such as substituting sodium with potassium (e.g. potassium glycocholate).

In several embodiments, the enhancing agent (e.g., absorption enhancer) in the IN pharmaceutical formulation is a dihydroxy conjugate (or a salt thereof). Exemplary embodiments of dihydroxy conjugates of bile acids that may be used in the intranasal formulations disclosed herein include tauroursodeoxycholate (TUDC), taurohyodeoxycholate (THDC), glycohyodeoxycholate (GHDC), glycochenodeoxycholate (GCDC), taurodeoxycholate (TDC), taurochenodeoxycholate (TCDC), glycodeoxychoate (GDC), glycoursodeoxycholate (GUDC), or a combination of any of the foregoing.

Exemplary embodiments of dihydroxy conjugates of bile salts that may be used in the intranasal formulations disclosed herein include sodium tauroursodeoxycholate (STUDC), sodium taurohyodeoxycholate (STHDC), sodium glycohyodeoxycholate (SGHDC), sodium glycochenodeoxycholate (SGCDC), taurodeoxycholate (TDC), sodium taurodeoxycholate (STDC), sodium taurochenodeoxycholate (STCDC), sodium glycodeoxychoate (SGDC), sodium glycoursodeoxycholate (SGUDC), or a combination of any of the foregoing. Other suitable forms of salts that may be used in the intranasal formulations disclosed herein are possible, such as substituting sodium with potassium (e.g. potassium tauroursodeoxycholate).

In several embodiments, the enhancing agent (e.g., absorption enhancer) in the IN pharmaceutical formulation is a unconjugated bile acid (or a salt thereof). Exemplary embodiments of unconjugated forms of bile acids that may be used in the intranasal formulations disclosed herein include cholate, deoxycholate (DC), chenodeoxycholate (CDC), or a combination of any of the foregoing.

Exemplary embodiments of unconjugated forms of bile salts that may be used in the intranasal formulations disclosed herein include sodium cholate (SC), sodium deoxycholate (SDC), sodium chenodeoxycholate (SCDC), or a combination of any of the foregoing. Other suitable forms of salts that may be used in the intranasal formulations disclosed herein are possible, such as substituting sodium with potassium (e.g. potassium cholate). The bile salts of the present disclosure are not limited to those described above, and may include any other suitable bile salts.

In several embodiments, the bile acids/salts are configured to aggregate and/or to form micelles in concentrations above a critical micelle concentration (CMC). In several embodiments, by forming micelles, bile acids/salts can facilitate transcellular passage and enhance absorption through the nasal mucosa. In several embodiments, the bile acid and/or bile salt is provided at a concentration above its CMC. CMC values of certain exemplary bile salts are: sodium taurocholate (STC): CMC is ˜8 mM, sodium cholate (SC): CMC is ˜4 mM, sodium lithocholate (SLC): CMC is ˜1 mM, sodium glycocholate (SGC): CMC is ˜2-5 mM, sodium taurochenodeoxycholate (STCDC): CMC is ˜2.5-3 mM. In several embodiments, the bile acid of the has a CMC of equal to or at least about: 1 mM, 2 mM, 3 mM, 4 mM, 6 mM, 8 mM, 10 mM, or ranges including and/or spanning the aforementioned values.

In several embodiments of the IN epinephrine pharmaceutical formulation, the bile acid, or the pharmaceutically acceptable salt thereof, is present at a concentration of equal to or less than about: 1 mg/mL, 3 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, or ranges including and/or spanning the aforementioned values. For example, in several embodiments, the IN epinephrine pharmaceutical formulation comprises a bile acid or a salt thereof at a concentration ranging from 5.0 mg/mL to 15 mg/mL, 6 mg/mL to 14 mg/mL, 8 mg/mL to 12 mg/mL, 7 mg/mL to 9 mg/mL, etc.

In several embodiments, the IN epinephrine pharmaceutical formulation comprises the bile acid, or the pharmaceutically acceptable salt thereof, at a molarity of equal to or less than about: 0.007 M, 0.01 M, 0.012 M, 0.014 M, 0.016 M, 0.018 M, 0.019 M, 0.020 M, 0.022 M, 0.025 M, or ranges including and/or spanning the aforementioned values. For example, in several embodiments, the IN epinephrine pharmaceutical formulation comprises bile acid or a salt thereof at a molarity ranging from 0.007 M to 0.022 M, 0.012 M to 0.020 M, 0.016 M to 0.025 M, 0.014 M to 0.019 M, etc.

In several embodiments of the IN epinephrine pharmaceutical formulations, the bile acid, or the salt thereof, is present at a concentration of 1.0 mg/mL to 15.0 mg/mL, or any concentration range subsumed therein, including but not limited to, 1.0 mg/mL to 12.5 mg/mL, 1.0 mg/mL to 10 mg/mL, 5.0 mg/mL to 11.0 mg/mL, 6.0 mg/mL to 13.0 mg/mL, 7.0 mg/mL to 12.0 mg/mL, 7.0 mg/mL to 9.0 mg/mL, 7.5 mg/mL to 9.5 mg/mL, 7.5 mg/mL to 8.5 mg/mL, 7.0 mg/mL to 9.0 mg/mL, or 7.0 mg/mL to 8.0 mg/mL. In several embodiments of the IN epinephrine pharmaceutical formulations, the bile acid, or the salt thereof, is present at a concentration of equal to or at least about: 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, 12.0 mg/mL, 12.5 mg/mL, 13.0 mg/mL, 13.5 mg/mL, 14.0 mg/mL, 14.5 mg/mL, 15.0 mg/mL, or ranges including and/or spanning the aforementioned values.

In several embodiments of the IN epinephrine pharmaceutical formulations, a bile acid, or the salt thereof, is present at a concentration of 5.0 mg/mL to 13.0 mg/mL. In several embodiments of the IN epinephrine pharmaceutical formulations, the bile acid, or the salt thereof, is present at a concentration of equal to or less than about: 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, 12.0 mg/mL, 12.5 mg/mL, 13.0 mg/mL, or ranges including and/or spanning the aforementioned values.

In several embodiments of the IN epinephrine pharmaceutical formulations, a bile acid, or the salt thereof, is present at a concentration of 7.0 mg/mL to 9.0 mg/mL. In several embodiments of the IN epinephrine pharmaceutical formulations, the bile acid, or the salt thereof, is present at a concentration of equal to or less than about: 7.0 mg/mL, 7.5 mg/mL, 8.0 mg/mL, 8.5 mg/mL, 9.0 mg/mL, or ranges including and/or spanning the aforementioned values.

In several embodiments, a dose of the pharmaceutical formulation comprises a bile acid, or a pharmaceutically acceptable salt thereof, in an amount ranging from 0.1 mg to 1.8 mg, or any amount range subsumed therein, including but not limited to, 0.6 mg to 1.3 mg, 0.5 mg to 1.1 mg, 0.7 mg to 1.2 mg, 0.7 mg to 0.9 mg, 0.75 mg to 0.95 mg, 0.75 mg to 0.85 mg, 0.70 mg to 0.90 mg, 0.70 mg to 0.80 mg, 1.0 mg to 1.4 mg, 0.9 mg to 1.3 mg, 1.0 mg to 1.4 mg, or 0.9 mg to 1.8 mg. In several embodiments, a dose of the pharmaceutical formulation comprises a bile acid, or a pharmaceutically acceptable salt thereof, in an amount of equal to or at least about: 0.10 mg, 0.15 mg, 0.20 mg, 0.25 mg, 0.30 mg, 0.35 mg, 0.40 mg, 0.45 mg, 0.50 mg, 0.55 mg, 0.60 mg, 0.65 mg, 0.70 mg, 0.75 mg, 0.80 mg, 0.85 mg, 0.90 mg, 0.95 mg, 1.00 mg, or ranges including and/or spanning the aforementioned values. In several embodiments, a dose of the pharmaceutical formulation comprises a bile acid, or a pharmaceutically acceptable salt thereof, in an amount of equal to or less than about: 0.80 mg, 0.85 mg, 0.90 mg, 0.95 mg, 1.00 mg, 1.05 mg, 1.10 mg, 1.15 mg, 1.20 mg, 1.25 mg, 1.30 mg, 1.35 mg, 1.40 mg, 1.45 mg, 1.50 mg, or ranges including and/or spanning the aforementioned values.

In several embodiments, a dose of the pharmaceutical formulation comprises a bile acid, or a pharmaceutically acceptable salt thereof, in an amount ranging from 0.5 mg to 1.3 mg. In several embodiments, a dose of the pharmaceutical formulation comprises a bile acid, or a pharmaceutically acceptable salt thereof, in an amount equal to or at least about: 0.50 mg, 0.55 mg, 0.60 mg, 0.65 mg, 0.70 mg, 0.75 mg, 0.80 mg, 0.85 mg, 0.90 mg, 0.95 mg, 1.00 mg, 1.05 mg, 1.10 mg, 1.15 mg, 1.20 mg, 1.25 mg, 1.30 mg, or ranges including and/or spanning the aforementioned values. In several embodiments of IN epinephrine pharmaceutical formulations, the bile acid, or the salt thereof, is present in a dose amount of equal to or less than about: 0.80 mg, 0.85 mg, 0.90 mg, 0.95 mg, 1.00 mg, 1.05 mg, 1.10 mg, 1.15 mg, 1.20 mg, 1.25 mg, 1.30 mg, or ranges including and/or spanning the aforementioned values.

In several embodiments, a dose of the pharmaceutical formulation comprises a bile acid, or a pharmaceutically acceptable salt thereof, in an amount ranging from 0.7 mg to 0.9 mg. In several embodiments, a dose of the pharmaceutical formulation comprises a bile acid, or a pharmaceutically acceptable salt thereof, in an amount equal to or at least about 0.70 mg, 0.75 mg, 0.80 mg, 0.85 mg, 0.90 mg, or ranges including and/or spanning the aforementioned values. In several embodiments of IN epinephrine pharmaceutical formulations, the bile acid, or the salt thereof, is present in a dose amount of equal to or less than about: 0.7.0 mg, 0.80 mg, 0.85 mg, 0.90 mg, 0.95 mg, or ranges including and/or spanning the aforementioned values.

In several embodiments, the IN epinephrine pharmaceutical formulation comprises epinephrine, or a pharmaceutically acceptable salt thereof, at a concentration of 1.0 mg/mL to 25.0 mg/mL, an absorption enhancer comprising a bile acid, or a salt thereof, at a concentration of 1.0 mg/mL to 15.0 mg/mL (or 5.0 mg/mL to 13.0 mg/mL), the pharmaceutical formulation has a pH of 2.2 to 5.0, and the pharmaceutical formulation is configured for IN delivery.

In several embodiments, the IN epinephrine pharmaceutical formulation comprises epinephrine, or a pharmaceutically acceptable salt thereof, present in a dose amount of 0.1 mg to 2.5 mg, an absorption enhancer comprising a bile acid, or a salt thereof, present in a dose amount of 0.1 mg to 1.5 mg (or 0.5 mg to 1.3 mg), the pharmaceutical formulation has a pH of 2.2 to 5.0, and the pharmaceutical formulation is configured for IN delivery.

### Sodium Taurocholate (STC)

As an exemplary embodiment of a trihydroxy conjugate bile salt, sodium taurocholate (STC) is a trihydroxy conjugate bile salt that has the molecular formula C26H44NNaO7S and a molecular weight (M.W.) of 537.7 g/mol. In several embodiments, the bile acid salt of the IN pharmaceutical formulation is STC. The chemical structure of STC is shown below:

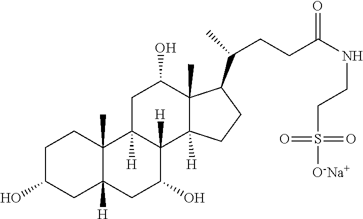

STC is an ionic amphiphilic compound with a steroid skeleton. It belongs to the family of endogenous bile salts, important for multiple physiological functions including lipid transport of nutrients and drugs across hydrophobic barriers through the process of solubilization. As shown by the chemical structure of STC, STC has a hydrophobic portion that includes the steroid portion, and a hydrophilic portion. STC has a critical micelle concentration (CMC) of about 4 mg/mL.

In several embodiments, as disclosed elsewhere herein, the bile salt is an STC. In several embodiments, the STC is an STC hydrate. In several embodiments, the STC or STC hydrate may be present in any amount or concentration disclosed elsewhere herein (e.g., at any amount or concentration provided for a bile acid salt or pharmaceutically acceptable bile acid salt). In several embodiments of the IN epinephrine pharmaceutical formulations, the STC is present at a concentration of 1.0 mg/mL to 15.0 mg/mL, or any concentration range subsumed therein, including but not limited to, 1.0 mg/mL to 12.5 mg/mL, 1.0 mg/mL to 10 mg/mL, 5.0 mg/mL to 11.0 mg/mL, 6.0 mg/mL to 13.0 mg/mL, 7.0 mg/mL to 12.0 mg/mL, 7.0 mg/mL to 9.0 mg/mL, 7.5 mg/mL to 9.5 mg/mL, 7.5 mg/mL to 8.5 mg/mL, 7.0 mg/mL to 9.0 mg/mL, or 7.0 mg/mL to 8.0 mg/mL. In several embodiments of the IN epinephrine pharmaceutical formulations, the STC is present at a concentration of equal to or at least about: 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, 12.0 mg/mL, 12.5 mg/mL, 13.0 mg/mL, 13.5 mg/mL, 14.0 mg/mL, 14.5 mg/mL, 15.0 mg/mL, or ranges including and/or spanning the aforementioned values. In several embodiments of the IN epinephrine pharmaceutical formulations, the STC is present at a concentration of equal to or less than about: 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, 12.0 mg/mL, 12.5 mg/mL, 13.0 mg/mL, 13.5 mg/mL, 14.0 mg/mL, 14.5 mg/mL, 15.0 mg/mL, or ranges including and/or spanning the aforementioned values.

In several embodiments, as disclosed elsewhere herein, the bile salt is an STC, such as an STC hydrate. In several embodiments of the IN epinephrine pharmaceutical formulations, the STC is present at a concentration of 5.0 mg/mL to 12.0 mg/mL. In several embodiments of the IN epinephrine pharmaceutical formulations, the STC is present at a concentration of equal to or at least about: 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, or 12.0 mg/mL, or ranges including and/or spanning the aforementioned values. In several embodiments of the IN epinephrine pharmaceutical formulations, the STC is present at a concentration of equal to or less than about: 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, or 12.0 mg/mL, or ranges including and/or spanning the aforementioned values.

In several embodiments, as disclosed elsewhere herein, the bile salt is an STC, such as an STC hydrate. In several embodiments of the IN epinephrine pharmaceutical formulations, the STC is present at a concentration of 7.0 mg/mL to 9.0 mg/mL. In several embodiments of the IN epinephrine pharmaceutical formulations, the STC is present at a concentration of equal to or at least about: 7.0 mg/mL, 7.5 mg/mL, 8.0 mg/mL, 8.5 mg/mL, 9.0 mg/mL or ranges including and/or spanning the aforementioned values.

In several embodiments, a dose of the pharmaceutical formulation comprises STC in an amount ranging from 0.1 mg to 1.5 mg, or any amount range subsumed therein, including but not limited to, 0.6 mg to 1.3 mg, 0.5 mg to 1.1 mg, 0.7 mg to 1.2 mg, 0.7 mg to 0.9 mg, 0.75 mg to 0.95 mg, 0.75 mg to 0.85 mg, 0.7 mg to 0.9 mg, or 0.7 mg to 0.8 mg. In several embodiments, a dose of the pharmaceutical formulation comprises STC in an amount equal to or at least about: 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, about 1.5 mg, or ranges including and/or spanning the aforementioned values. In several embodiments, a dose of the pharmaceutical formulation comprises STC in an amount equal to or less than about: 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, about 1.5 mg, or ranges including and/or spanning the aforementioned values.

In several embodiments, a dose of the pharmaceutical formulation comprises STC in an amount ranging from 0.5 mg to 1.2 mg. In several embodiments, a dose of the pharmaceutical formulation comprises STC in an amount of equal to or at least about: 0.50 mg, 0.55 mg, 0.60 mg, 0.65 mg, 0.70 mg, 0.75 mg, 0.80 mg, 0.85 mg, 0.90 mg, 0.95 mg, 1.00 mg, 1.05 mg, 1.10 mg, 1.15 mg, 1.20 mg, or ranges including and/or spanning the aforementioned values.

In several embodiments, a dose of the pharmaceutical formulation comprises STC in an amount ranging from 0.7 mg to 0.9 mg. In several embodiments, a dose of the pharmaceutical formulation comprises STC in an amount of equal to or at least about: 0.70 mg, 0.75 mg, 0.80 mg, 0.85 mg, 0.90 mg, or ranges including and/or spanning the aforementioned values.

As disclosed elsewhere herein, in several embodiments, the IN epinephrine pharmaceutical formulation comprises epinephrine, or a pharmaceutically acceptable salt thereof, present at a concentration of 1.0 mg/mL to 25.0 mg/mL, and an absorption enhancer comprising a bile salt present at a concentration of 1.0 mg/mL to 15.0 mg/mL (or 5.0 mg/mL to 13.0 mg/mL), wherein the bile salt is STC. In several embodiments, the pharmaceutical formulation has a pH of 2.2 to 5.0. In several embodiments, the and wherein the pharmaceutical formulation is configured for IN delivery.

In several embodiments, the IN epinephrine pharmaceutical formulation comprises epinephrine, or a pharmaceutically acceptable salt thereof, present in a dose amount of 0.1 mg to 2.5 mg, an absorption enhancer comprising a bile salt present in a dose amount of 0.1 mg to 1.5 mg (or 0.5 mg to 1.3 mg), wherein the bile salt is STC. In several embodiments, the pharmaceutical formulation has a pH of 2.2 to 5.0. In several embodiments, the pharmaceutical formulation is for IN delivery.

### Sodium Taurochenodeoxycholate (STCDC).

As another exemplary embodiment of a dihydroxy conjugate bile salt, sodium taurochenodeoxycholate (STCDC) is a dihydroxy conjugate bile salt that has the molecular formula C₂₆H₄₄NNaO₆S and a molecular weight (M.W.) of 521.7 g/mol. In several embodiments, the bile acid salt of the IN pharmaceutical formulation is STCDC. The chemical structure of STCDC is shown below:

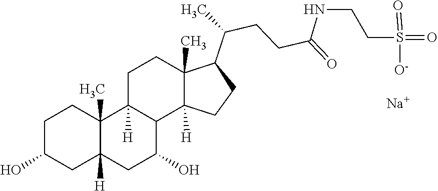

In several embodiments, the bile salt is an STCDC. In several embodiments of the IN epinephrine pharmaceutical formulations, the STCDC is present at a concentration of 1.0 mg/mL to 15.0 mg/mL, or any concentration range subsumed therein, including but not limited to, 1.0 mg/mL to 12.5 mg/mL, 1.0 mg/mL to 10 mg/mL, 5.0 mg/mL to 11.0 mg/mL, 6.0 mg/mL to 13.0 mg/mL, 7.0 mg/mL to 12.0 mg/mL, 7.0 mg/mL to 9.0 mg/mL, 7.5 mg/mL to 9.5 mg/mL, 7.5 mg/mL to 8.5 mg/mL, 7.0 mg/mL to 9.0 mg/mL, or 7.0 mg/mL to 8.0 mg/mL. In several embodiments of the IN epinephrine pharmaceutical formulations, the STCDC is present at a concentration of equal to or at least about: 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, 12.0 mg/mL, 12.5 mg/mL, 13.0 mg/mL, 13.5 mg/mL, 14.0 mg/mL, 14.5 mg/mL, 15.0 mg/mL, or ranges including and/or spanning the aforementioned values. In several embodiments of the IN epinephrine pharmaceutical formulations, the STCDC is present at a concentration of equal to or less than about: 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, 12.0 mg/mL, 12.5 mg/mL, 13.0 mg/mL, 13.5 mg/mL, 14.0 mg/mL, 14.5 mg/mL, 15.0 mg/mL, or ranges including and/or spanning the aforementioned values.

In several embodiments of the IN epinephrine pharmaceutical formulations, the STCDC is present at a concentration of 2.0 mg/mL to 10.0 mg/mL. In several embodiments of the IN epinephrine pharmaceutical formulations, the STCDC is present at a concentration of equal to or at least about: 2.0 mg/ml; 2.5 mg/ml; 3.0 mg/ml; 3.5 mg/ml; 4.0 mg/ml; 4.5 mg/ml; 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, or ranges including and/or spanning the aforementioned values. In several embodiments of the IN epinephrine pharmaceutical formulations, the STCDC is present at a concentration of equal to or less than about: 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, or 12.0 mg/mL, or ranges including and/or spanning the aforementioned values.

In several embodiments, a dose of the pharmaceutical formulation comprises STCDC in an amount ranging from 0.1 mg to 1.5 mg, or any amount range subsumed therein, including but not limited to, 0.6 mg to 1.3 mg, 0.5 mg to 1.1 mg, 0.7 mg to 1.2 mg, 0.7 mg to 0.9 mg, 0.75 mg to 0.95 mg, 0.75 mg to 0.85 mg, 0.7 mg to 0.9 mg, or 0.7 mg to 0.8 mg. In several embodiments, a dose of the pharmaceutical formulation comprises STCDC in an amount equal to or at least about: 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, about 1.5 mg, or ranges including and/or spanning the aforementioned values.

In several embodiments, a dose of the pharmaceutical formulation comprises STCDC in an amount ranging from 0.2 mg to 1.0 mg. In several embodiments, a dose of the pharmaceutical formulation comprises STCDC in an amount equal to or less than about: 0.2 mg; 0.25 mg; 0.3 mg; 0.35 mg; 0.4 mg; 0.45 mg; 0.5 mg; 0.55 mg; 0.6 mg; 0.65 mg; 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, about 1.5 mg, or ranges including and/or spanning the aforementioned values.

As disclosed elsewhere herein, in several embodiments, the IN epinephrine pharmaceutical formulation comprises epinephrine, or a pharmaceutically acceptable salt thereof, present at a concentration of 1.0 mg/mL to 25.0 mg/mL, an absorption enhancer comprising a bile salt present at a concentration of 1.0 mg/mL to 15.0 mg/mL (or 5.0 mg/mL to 13.0 mg/mL), wherein the bile salt is STCDC. In several embodiments, the pharmaceutical formulation has a pH of 2.2 to 5.0. In several embodiments, the pharmaceutical formulation is for IN delivery.

In several embodiments, the IN epinephrine pharmaceutical formulation comprises epinephrine, or a pharmaceutically acceptable salt thereof, present in a dose amount of 0.1 mg to 2.5 mg, and an absorption enhancer comprising a bile salt present in a dose amount of 0.1 mg to 1.5 mg (or 0.5 mg to 1.3 mg). In several embodiments, the bile salt is STCDC. In several embodiments, the pharmaceutical formulation has a pH of 2.2 to 5.0. In several embodiments, the pharmaceutical formulation is for IN delivery.

### Bile Acid/Salt Enhancement Factor (EF)

The absorption enhancement effectiveness of the bile acid/salt can be quantified with reference to the following equation:

$$EF ( S ) = \frac{\overline{R} ( S )}{\overline{R} ( 0 )}$$

where EF(S) is the Bile Acid/Salt Enhancement Factor (“EF”)

^{R̅}(S) is an average of the dose-normalized bioavailability (DN-RBA) for X (3 PK parameters: AUC₀₋₃₀ₘᵢₙ, AUC_{0-∞}, and Cₘₐₓ) by the IN route at a given Bile Acid/Salt concentration S. Where simply referred to as the “Enhancement Factor” or “EF” without a PK subscript, what is meant is the enhancement factor calculated using AUC₀₋₃₀ₘᵢₙ, AUC_{0-∞}, and Cₘₐₓ to calculate the dose normalized bioavailability. However, other AUC measures may be used to provide various other enhancement factors. These other enhancement factors are reported herein using a subscript and listing the PK parameters used to calculate the enhancement factor (e.g., “EF_{PK1, PK2, PK3}”). For instance, an enhancement factor calculated using AUC₀₋₃₀ₘᵢₙ, AUC₀₋₁₈₀ₘᵢₙ, and Cₘₐₓ is reported as EF_{AUC0-30/AUC0-180/Cmax} in Example 1. If an enhancement factor is reported as EF without a subscript, what is meant is the enhancement factor using AUC₀₋₃₀ₘᵢₙ, AUC_{0-∞}, and Cₘₐₓ to calculate the dose-normalized bioavailability. Here, 3 parameters are used for purposes of illustration but X can be any number of parameters, including 1 parameter or more.
^{R̅}(0) has the same definition at S=0, by the IN route.

Additionally, the dose-normalized relative bioavailability (DN-RBA) is defined as follows:

$${R}_{X} ( S ) = \frac{{X}_{IN} ( {d}_{IN ,} S ) \frac{1}{{d}_{IN}}}{{X}_{IM} ( {d}_{IM} ) \frac{1}{{d}_{IM}}}$$

where Rx is the DN-RBA for PK parameters X;

S is the concentration of Bile Acid/Salt (i.e. STC) used in the IN epinephrine formulations;

As noted above, X are AUC₀₋₃₀ₘᵢₙ, AUC_{0-∞}, and Cₘₐₓ, note that AUC₀₋₃₀ₘᵢₙ, AUC_{0-∞}, and Cₘₐₓ are used for illustrative purposes, other PK parameters can also be assessed. For example, in calculating an EF_{0-30/0-180/Cmax}, X are partial AUC, AUC₀₋₃₀ₘᵢₙ, AUC₀₋₁₈₀ₘᵢₙ, and Cₘₐₓ.

d_{IM} and d_{IN} are doses delivered by IM and IN routes, respectively. As an example, the IM can be the 1 mg/mL epinephrine IM injection.

Example 1 will demonstrate the application of these principles to calculate EF_{AUC0-30/AUC0-180/Cmax} and Example 6 will demonstrate the application of these principles to calculate EF (e.g., EF_{AUC0-30/AUC0-∞/Cmax}). Example 1 will show that the IN epinephrine formulations comprising the bile acid, or the salt thereof, as the absorption enhancer provided an Enhancement Factor_{0-30/0-180/Cmax} in a range of 1 to 23. In several embodiments, the EF is in a range of 1 to 23 based on an intranasal delivery (IN) v. intramuscular injection (IM) averaged pharmacokinetic (PK) results for AUC₀₋₃₀ₘᵢₙ, AUC_{0-∞}, and Cₘₐₓ. In several embodiments, the EF_{0-30/0-180/Cmax} is in a range of 1 to 23 based on an intranasal delivery (IN) v. intramuscular injection (IM) averaged pharmacokinetic (PK) results for AUC₀₋₃₀ₘᵢₙ, AUC₀₋₁₈₀ₘᵢₙ, and Cₘₐₓ.

In several embodiments, the bile acid, or the salt thereof, provides an EF of at least 4, wherein the EF is determined based on ^{R̅}(S)/^{R̅}(0), where ^{R̅}(S) is an average of a dose-normalized relative bioavailability (DN-RBA) of the pharmaceutical formulation relative to an IM injection having same API for two or more pharmacokinetic (PK) parameters, and ^{R̅}(0) is an average of DN-RBA of a pharmaceutical formulation without an absorption enhancer relative to an IM injection having same API for the two or more PK parameters. In several embodiments, the EF achieved using a bile acid or salt thereof is equal to or at least about: 4, 6, 8, 9, 10, 11, 12, 13, 14, 15, or ranges including and/or spanning the aforementioned values.

In several embodiments, the bile acid, or the salt thereof, provides an enhancement factor in a range of 1 to 23 or any range subsumed therein, including, but not limited to, 2 to 23, 3 to 23, 4 to 23, 5 to 23, 6 to 23, 7 to 23, 8 to 23, 9 to 23, 10 to 23, 11 to 23, 12 to 23, 13 to 23, 14 to 23, 15 to 23, 16 to 23, 17 to 23, 18 to 23, 19 to 23, 20 to 23, 21 to 23, or 22 to 23. In several embodiments, the bile acid, or the salt thereof, provides an enhancement factor of at least 2, 3, 4, 5, 6, 7, 8, 9, 10, 11, 12, 13, 14, 15, 16, 17, 18, 19, 20, 21, 22, or 23. In several embodiments of the IN formulations, the absorption enhancer comprising the bile acid, or the salt thereof, provides an enhancement factor EF of more than 23.

In several embodiments, the enhancement factor is provided as EF_{PK1}, EF_{PK1, PK2}, or EF_{PK1, PK2, PK3}, where each of PK1, PK2, and PK3 are independently selected from a pharmacokinetic parameter. In several embodiments, PK1 is selected from Cₘₐₓ, tₘₐₓ, AUC_{0-t*}, AUC_{0-10 min}, AUC₀₋₃₀ₘᵢₙ, AUC_{0-180 min}, AUC_{0-6 hr}, and AUC_{0-∞}. In several embodiments, where present, PK2 is selected from Cₘₐₓ, tₘₐₓ, AUC_{0-t*}, AUC_{0-10 min}, AUC₀₋₃₀ₘᵢₙ, AUC_{0-180 min}, AUC₀₋₆ₕᵣ, and AUC_{0-∞}. In several embodiments, where present, PK3 is selected from is selected from Cₘₐₓ, tₘₐₓ, AUC_{0-t*}, AUC_{0-10 min}, AUC₀₋₃₀ₘᵢₙ, AUC_{0-180 min}, AUC₀₋₆ₕᵣ, and AUC_{0-∞}. In several embodiments, where present in a given enhancement factor, each of PK1 and PK2 or PK1, PK2, and PK3 are different. In several embodiments, the EF_{PK1}, EF_{PK1, PK2}, or EF_{PK1, PK2, PK3} achieved using a bile acid or salt thereof is equal to or at least about: 4, 6, 8, 9, 10, 11, 12, 13, 14, 15, 20, or ranges including and/or spanning the aforementioned values. In several embodiments, the bile acid, or the salt thereof, provides an EF_{PK1}, EF_{PK1, PK2}, or EF_{PK1, PK2, PK3} in a range of 1 to 23 or any range subsumed therein, including, but not limited to, 2 to 23, 3 to 23, 4 to 23, 5 to 23, 6 to 23, 7 to 23, 8 to 23, 9 to 23, 10 to 23, 11 to 23, 12 to 23, 13 to 23, 14 to 23, 15 to 23, 16 to 23, 17 to 23, 18 to 23, 19 to 23, 20 to 23, 21 to 23, or 22 to 23. In several embodiments, the bile acid, or the salt thereof, provides an EF_{PK1}, EF_{PK1}, PK2, or EF_{PK1, PK2, PK3} of at least 2, 3, 4, 5, 6, 7, 8, 9, 10, 11, 12, 13, 14, 15, 16, 17, 18, 19, 20, 21, 22, or 23. In several embodiments of the IN formulations, the absorption enhancer comprising the bile acid, or the salt thereof, provides an EF_{PK1}, EF_{PK1, PK2}, or EF_{PK1, PK2, PK3} of more than 23.

In several embodiments, the PK parameter (e.g., PK1) is selected from the group consisting of Cₘₐₓ, tₘₐₓ, AUC_{0-t*}, AUC_{0-10 min}, AUC₀₋₃₀ₘᵢₙ, AUC_{0-180 min}, AUC₀₋₆ₕᵣ, and AUC_{0-∞}. In several embodiments, the two or more PK parameters (e.g., PK1 and PK2; PK1, PK2, and PK3, etc.) are selected from the group consisting of Cₘₐₓ, tₘₐₓ, AUC_{0-t*}, AUC_{0-10 min}, AUC₀₋₃₀ₘᵢₙ, AUC_{0-180 min}, AUC₀₋₆ₕᵣ, and AUC_{0-∞}. In several embodiments, the two or more PK parameters include two or more of Cₘₐₓ, AUC_{0-t*}, and AUC₀₋₃₀ₘᵢₙ. In several embodiments, the two or more PK parameters include tₘₐₓ, AUC_{0-t*}, and AUC₀₋₆ₕᵣ. In several embodiments, the two or more PK parameters include Cₘₐₓ, AUC₀₋₃₀ₘᵢₙ, and AUC₀₋₆ₕᵣ, and AUC_{0-∞}. In several embodiments, the two or more PK parameters include Cₘₐₓ, AUC_{0-t*}, and AUC_{0-∞}. In several embodiments, the two or more PK parameters include AUC₀₋₃₀ₘᵢₙ, AUC₀₋₁₈₀ₘᵢₙ, and Cₘₐₓ. In other embodiments, the two or more PK parameters include AUC_{0-10 min}, AUC₀₋₁₅ₘᵢₙ, AUC₀₋₃₀ₘᵢₙ, AUC₀₋₄₅ₘᵢₙ, AUC₀₋₆₀ₘᵢₙ, AUC₀₋₇₅ₘᵢₙ, AUC₀₋₉₀ₘᵢₙ, AUC₀₋₁₀₀ₘᵢₙ, AUC₀₋₁₂₅ₘᵢₙ, AUC₀₋₁₈₀ₘᵢₙ, AUC₀₋₁₈₀ₘᵢₙ, AUC_{0-infinity}, Cₘₐₓ, tₘₐₓ, other suitable PK parameters, or any combination thereof.

### IN Epinephrine Pharmaceutical Formulations May Include Pharmaceutically Acceptable Excipients

The disclosed IN epinephrine pharmaceutical formulations further include one or more pharmaceutically acceptable excipients.

### pH Adjusting Or pH Stabilizing Agents

In several embodiments, the pH of the IN epinephrine pharmaceutical formulation is acidic. In several embodiments, the pH of the IN epinephrine pharmaceutical formulation is 2.2 to 5.0, or any pH range subsumed therein, including but not limited to, 3.0 to 4.5, 3.0 to 3.5, 3.5 to 4.0, 3.7 to 3.9, 3.75 to 3.85, 4.0 to 4.5, or 4.5 to 5.0. In several embodiments, the pH of the IN epinephrine pharmaceutical formulation is equal to or less than about: 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, or ranges including and/or spanning the aforementioned values. For example, in several embodiments, the pH of the IN epinephrine pharmaceutical formulation ranges from 3.2 to 4.5, 3.4 to 5.0, from 3.7 to 3.9, etc.

In several embodiments, the pH of the IN epinephrine pharmaceutical formulation is 3.7 to 3.9, including any range subsumed therein. In several embodiments, the pH of the IN epinephrine pharmaceutical formulation is equal to or less than about: 3.7, 3.8, 3.9, or ranges including and/or spanning the aforementioned values.

In several embodiments, the IN epinephrine pharmaceutical formulations further includes a buffer (e.g., a buffer system). In several embodiments, the buffer comprises one or more of citric acid, sodium citrate, sodium phosphate, or a combination thereof, but the present disclosure is not limited thereto. In several embodiments, the buffer system comprises an acid and its conjugate base. In several embodiments, the buffer can include a first buffer agent (e.g., an acid), such as citric acid, and a second buffer agent (e.g., a conjugate base), such as sodium citrate, thereby forming a buffer pair. In some embodiments, the acid (e.g., conjugate acid) is adipic acid, ammonium chloride, citric acid, acetic acid, formic acid, lactic acid, phosphoric acid, propionic acid, tartaric acid, combinations of the foregoing, or other acids. In several embodiments, the base (e.g., conjugate base) is acetate (e.g., sodium acetate, etc.), citrate (e.g., sodium citrate, etc.), bicarbonate (e.g., sodium bicarbonate, etc.), carbonate (e.g., sodium carbonate), lactate (e.g., sodium lactate, etc.), phosphate (e.g., sodium phosphate), combinations of the foregoing, or other bases. In several embodiments, the buffer is a phosphate buffer, an acetate buffer, or a citrate buffer. In several embodiments, the buffer is a citrate buffer. In several embodiments, the buffer is a MES hydrate or monohydrate buffer. In several embodiments, the buffer is a BIS TRIS buffer.

In several embodiments of the IN epinephrine pharmaceutical formulations, the buffer includes an acid (e.g., conjugate acid). In several embodiments, the acid (e.g., conjugate acid) is present at a concentration of 1.0 mg/mL to 8.0 mg/mL, or any concentration range subsumed therein, including but not limited to, 2.0 mg/mL to 7.0 mg/mL, 1.5 mg/mL to 6.5 mg/mL, 2.0 mg/mL to 7.0 mg/mL, or 3.0 mg/mL to 5.0 mg/mL. In several embodiments of the IN epinephrine pharmaceutical formulations, the acid (e.g., conjugate acid) is present at a concentration of equal to or less than about: 1.0 mg/mL, 2.0 mg/mL, 3.0 mg/mL, 4.0 mg/mL, 5.0 mg/mL, 6.0 mg/mL, 7.0 mg/mL, 8.0 mg/mL, or ranges including and/or spanning the aforementioned values. In several embodiments of the IN epinephrine pharmaceutical formulations, the buffer includes citric acid (or a citric acid source) present at a concentration of 1.0 mg/mL to 8.0 mg/mL, or any concentration range subsumed therein, including but not limited to, 2.0 mg/mL to 7.0 mg/mL, 1.5 mg/mL to 6.5 mg/mL, 2.0 mg/mL to 7.0 mg/mL, or 3.0 mg/mL to 5.0 mg/mL. In several embodiments of the IN epinephrine pharmaceutical formulations, the citric acid (or citric acid source) is present at a concentration of about: 1.0 mg/mL, 2.0 mg/mL, 3.0 mg/mL, 4.0 mg/mL, 5.0 mg/mL, 6.0 mg/mL, 7.0 mg/mL, 8.0 mg/mL, or ranges including and/or spanning the aforementioned values. In several embodiments of the IN epinephrine pharmaceutical formulations, the citric acid source is citric acid monohydrate.

In several embodiments of the IN epinephrine pharmaceutical formulations, the buffer includes a base (e.g., a conjugate base) present at a concentration of 1.0 mg/mL to 10.0 mg/mL, or any concentration range subsumed therein, including but not limited to, 5.0 mg/mL to 10.0 mg/mL, 2.0 mg/mL to 8.0 mg/mL, 4.0 mg/mL to 7.0 mg/mL, 7.0 mg/mL to 9.0 mg/mL, or any concentration range subsumed therein. In several embodiments of the IN epinephrine pharmaceutical formulations, the base (e.g., a conjugate base) is present at a concentration of equal to or less than about: 1.0 mg/mL, 2.0 mg/mL, 3.0 mg/mL, 4.0 mg/mL, 5.0 mg/mL, 6.0 mg/mL, 7.0 mg/mL, 8.0 mg/mL, 9.0 mg/mL, 10.0 mg/mL, or ranges including and/or spanning the aforementioned values. In several embodiments of the IN epinephrine pharmaceutical formulations, the buffer includes sodium citrate (or a sodium citrate source) present at a concentration of 1.0 mg/mL to 10.0 mg/mL, or any concentration range subsumed therein, including but not limited to, 5.0 mg/mL to 10.0 mg/mL, 2.0 mg/mL to 8.0 mg/mL, 4.0 mg/mL to 7.0 mg/mL, 7.0 mg/mL to 9.0 mg/mL, or any concentration range subsumed therein. In several embodiments of the IN epinephrine pharmaceutical formulations, the sodium citrate (or sodium citrate source) is present at a concentration about: 1.0 mg/mL, 2.0 mg/mL, 3.0 mg/mL, 4.0 mg/mL, 5.0 mg/mL, 6.0 mg/mL, 7.0 mg/mL, 8.0 mg/mL, 9.0 mg/mL, 10.0 mg/mL, or ranges including and/or spanning the aforementioned values. In several embodiments of the IN epinephrine pharmaceutical formulations, the sodium citrate source is a sodium citrate dihydrate. In several embodiments of the IN epinephrine pharmaceutical formulations, the buffer includes citric acid and sodium citrate.

In several embodiments of the IN epinephrine pharmaceutical formulations, the buffer includes citric acid in a concentration range of 3.0 mg/mL to 5.0 mg/mL and sodium citrate in a concentration range of 6.0 mg/mL to 10.0 mL. In several embodiments of the IN epinephrine pharmaceutical formulations, the buffer includes citric acid having a concentration of about 4.0 mg/mL and sodium citrate having a concentration of about 8.0 mg/mL.

In several embodiments, the IN epinephrine pharmaceutical formulation comprises a buffer (e.g., the acid and conjugate base; the conjugate acid and base pair; etc.), at a molarity of equal to or less than about: 0.01 M, 0.02 M, 0.03 M, 0.04 M, 0.05 M, 0.06 M, 0.07 M, 0.08 M, 0.09 M, 0.1 M, or ranges including and/or spanning the aforementioned values. For example, in several embodiments, the IN epinephrine pharmaceutical formulation comprises the buffer at a molarity ranging from 0.01 M to 0.1 M, 0.02 M to 0.08 M, 0.06 M to 0.1 M, 0.05 M to 0.2 M, etc. In several embodiments, the IN epinephrine pharmaceutical formulation comprises the buffer is a citrate buffer at a molarity of equal to or less than about: 0.01 M, 0.02 M, 0.03 M, 0.04 M, 0.05 M, 0.06 M, 0.07 M, 0.08 M, 0.09 M, 0.1 M, or ranges including and/or spanning the aforementioned values. In several embodiments, the IN epinephrine pharmaceutical formulation comprises the buffer is an acetate buffer at a molarity of equal to or less than about: 0.01 M, 0.02 M, 0.03 M, 0.04 M, 0.05 M, 0.06 M, 0.07 M, 0.08 M, 0.09 M, 0.1 M, or ranges including and/or spanning the aforementioned values.

### Preservatives

In several embodiments, the IN epinephrine pharmaceutical formulations further include a preservative. In several embodiments, the preservative is selected from the group consisting of chlorobutanol, parabens (e.g., methyl paraben), phenyl ethyl alcohol, benzalkonium chloride, benzoyl alcohol, meta-cresol, a combination thereof, or other preservatives. In several embodiments, the preservative is selected from the group consisting chlorobutanol, alcohol, benzalkonium chloride, benzethonium chloride, benzoic acid, benzyl alcohol, boric acid, bronopol, butylated hydroxyanisole (BHA), butylene glycol, butylparaben, calcium acetate, calcium chloride, calcium lactate, carbon dioxide, bentonite, cetrimide, cetylpyridinium chloride, chlorhexidine, chlorobutanol, chlorocresol, chloroxylenol, citric acid monohydrate, cresol, dimethyl ether, ethylparaben, glycerin, hexetidine, imidurea, magnesium trisilicate, isopropyl alcohol, lactic acid, methylparaben, monothioglycerol, parabens (methyl, ethyl and propyl), pentetic acid, phenol, phenoxyethanol, phenylethyl alcohol, phenylmercuric acetate, phenylmercuric borate, phenylmercuric nitrate, potassium benzoate, potassium metabisulfite, potassium sorbate, propionic acid, propyl gallate, propylene glycol, propylparaben, propylparaben sodium, sodium acetate, sodium benzoate, sodium borate, sodium lactate, sodium metabisulfite, sodium propionate, sodium sulfite, sorbic acid, sulfobutyletherb-cyclodextrin, sulfur dioxide, edetic acid, thimerosal, xylitol, and/or combinations of any of the foregoing.

In several embodiments, the preservative is present in the composition at a concentration of 1.0 mg/mL to 9.0 mg/mL, or any concentration range subsumed therein, including but not limited to, 3.0 mg/mL to 8.0 mg/mL, 4.0 mg/mL to 7.0 mg/mL, 4.5 mg/mL to 6.5 mg/mL, 4.0 mg/mL to 6.0 mg/mL, or 5.0 mg/mL to 6.0 mg/mL. In several embodiments, the IN epinephrine pharmaceutical formulation includes a preservative (or one or more preservatives) at a concentration of equal to or less than about: 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, or ranges including and/or spanning the aforementioned values.

In several embodiments, the IN epinephrine pharmaceutical formulations include chlorobutanol (or a chlorobutanol source) at a concentration of 1.0 mg/mL to 9.0 mg/mL, or any concentration range subsumed therein, including but not limited to, 3.0 mg/mL to 8.0 mg/mL, 4.0 mg/mL to 7.0 mg/mL, 4.5 mg/mL to 6.5 mg/mL, 4.0 mg/mL to 6.0 mg/mL, or 5.0 mg/mL to 6.0 mg/mL. In several embodiments, the IN epinephrine pharmaceutical formulation includes chlorobutanol at a concentration of equal to or less than about: 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, or ranges including and/or spanning the aforementioned values. In several embodiments of the IN epinephrine pharmaceutical formulations, the chlorobutanol source is chlorobutanol hemihydrate.

In several embodiments, the IN epinephrine pharmaceutical formulation comprises a preservative (e.g., chlorobutanol, chlorobutanol hemihydrate, etc.) at a molarity of equal to or less than about: 0.007 M, 0.01 M, 0.012 M, 0.014 M, 0.016 M, 0.018 M, 0.019 M, 0.020 M, 0.022 M, 0.025 M, or ranges including and/or spanning the aforementioned values. For example, in several embodiments, the IN epinephrine pharmaceutical formulation comprises a preservative (e.g., chlorobutanol, chlorobutanol hemihydrate, etc.) at a molarity ranging from 0.007 M to 0.022 M, 0.012 M to 0.020 M, 0.012 M to 0.018 M, 0.014 M to 0.019 M, etc.

In several embodiments of the IN epinephrine pharmaceutical formulation, the preservative is chlorobutanol present in a concentration range of 4.0 mg/mL to 7.0 mg/mL. In other embodiments of the IN epinephrine pharmaceutical formulations, the preservative is chlorobutanol present at a concentration of about 5.5 mg/mL.

Metal Complexing Agents and/or Stabilizing Agent

In several embodiments, the epinephrine pharmaceutical formulation includes a metal complexing agent. In several embodiments, the metal complexing agent is ethylenediaminetetraacetic acid (EDTA), disodium edetate dihydrate (disodium EDTA), diethylenetriamine pentaacetic acid (DTPA), or any other suitable metal complexing agent, or a combination thereof, but the present disclosure is not limited thereto. In several embodiments, the IN epinephrine pharmaceutical formulations include a metal complexing agent (e.g., EDTA, disodium EDTA, etc.) at a concentration of 0.01 mg/mL to 0.10 mg/mL, or any concentration range subsumed therein, including but not limited to, 0.01 mg/mL to 0.08 mg/mL, 0.01 mg/mL to 0.05 mg/mL, 0.01 mg/mL to 0.03 mg/mL, or 0.01 mg/mL to 0.02 mg/mL. In several embodiments, the IN epinephrine pharmaceutical formulation includes a metal complexing agent (e.g., EDTA, disodium EDTA, etc.) at a concentration of equal to or less than about: 0.005 mg/mL, 0.01 mg/mL, 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.10 mg/mL, or ranges including and/or spanning the aforementioned values.

In several embodiments of the IN epinephrine pharmaceutical formulation, the metal complexing agent is disodium EDTA present in a concentration range of 0.005 mg/mL to 0.05 mg/mL. In several embodiments of the IN epinephrine pharmaceutical formulations, the metal complexing agent is disodium EDTA present at a concentration of about 0.02 mg/mL.

In several embodiments, the IN epinephrine pharmaceutical formulation comprises a metal complexing agent (e.g., EDTA, disodium EDTA) at a molarity of equal to or less than about: 1×10⁻⁵ M, 2.5×10⁻⁵ M, 5.0×10⁻⁵ M, 6.0×10⁻⁵ M, 7.5×10⁻⁵ M, 1.0×10⁻⁴ M, or ranges including and/or spanning the aforementioned values. For example, in several embodiments, the IN epinephrine pharmaceutical formulation comprises a metal complexing agent (e.g., EDTA, disodium EDTA) at a molarity ranging from 1×10⁻⁵ M to 1×10⁻⁴ M, 5.0×10⁻⁵ M to 6.0×10⁻⁵ M, etc.

### Tonicity Agents

In several embodiments, the IN epinephrine pharmaceutical formulation comprises one or more tonicity agents. In several embodiments, the tonicity agent may include or is sodium chloride, dextrose, glucose, glycerin, cellulose, mannitol, polysorbate, propylene glycol, sodium iodide, or a combination thereof, but the present disclosure is not limited thereto. In several embodiments, the tonicity agent is present at a concentration of 1.0 mg/mL to 5.0 mg/mL, or any concentration range subsumed therein, including but not limited to, 1.0 mg/mL to 4.0 mg/mL, 1.0 mg/mL to 3.0 mg/mL, 2.0 mg/mL to 5.0 mg/mL, 2.0 mg/mL to 4.0 mg/mL. In several embodiments, the tonicity agent is present a concentration of equal to or less than about: 0.5 mg/mL, 1.0 mg/mL, 2.0 mg/mL, 3.0 mg/mL, 4.0 mg/mL, 5.0 mg/mL, or ranges including and/or spanning the aforementioned values.

In several embodiments of the IN epinephrine pharmaceutical formulation, the tonicity agent is sodium chloride and is present at a concentration of 1.0 mg/mL to 5.0 mg/mL, or any concentration range subsumed therein, including but not limited to, 1.0 mg/mL to 4.0 mg/mL, 1.0 mg/mL to 3.0 mg/mL, 2.0 mg/mL to 5.0 mg/mL, 2.0 mg/mL to 4.0 mg/mL. In several embodiments, the IN epinephrine pharmaceutical formulations include sodium chloride present a concentration of equal to or less than about: 0.5 mg/mL, 1.0 mg/mL, 2.0 mg/mL, 3.0 mg/mL, 4.0 mg/mL, 5.0 mg/mL, or ranges including and/or spanning the aforementioned values.

In several embodiments of the IN epinephrine pharmaceutical formulation, the tonicity agent is sodium chloride present in a concentration range of 1.0 mg/mL to 5.0 mg/mL. In several embodiments of the IN epinephrine pharmaceutical formulation, the tonicity agent is sodium chloride present at a concentration of about 1.50 mg/mL, 1.75 mg/mL, 2.00 mg/mL, 2.25 mg/mL, 2.50 mg/mL, 2.75 mg/mL or 3.00 mg/mL.

In several embodiments, the IN epinephrine pharmaceutical formulation comprises the tonicity agent at a molarity of equal to or less than about: 0.01 M, 0.02 M, 0.04 M, 0.05 M, 0.06 M, 0.07 M, 0.080 M, 0.10 M, or ranges including and/or spanning the aforementioned values. For example, in several embodiments, the IN epinephrine pharmaceutical formulation comprises the tonicity agent at a molarity ranging from 0.01 M to 0.10 M, 0.02 M to 0.04 M, 0.01 M to 0.05 M.

### Antioxidants

In several embodiments, the IN epinephrine pharmaceutical formulation comprises an antioxidant. In several embodiments, the antioxidant is selected from the group consisting of sodium metabisulfite, sodium bisulfate, other sulfites, butylated hydroxytoluene, tocopherol, or a combination thereof, but the present disclosure is not limited thereto. In several embodiments, the IN epinephrine pharmaceutical formulations include the antioxidant at a concentration of 0.1 mg/mL to 1.0 mg/mL, or any concentration range subsumed therein, including but not limited to, 0.1 mg/mL to 0.9 mg/mL, 0.1 mg/mL to 0.8 mg/mL, 0.1 mg/mL to 0.5 mg/mL, 0.2 mg/mL to 0.5 mg/mL, or 0.2 mg/mL to 0.4 mg/mL. In several embodiments, the IN epinephrine pharmaceutical formulations includes the antioxidant at a concentration of equal to or less than about: 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, or ranges including and/or spanning the aforementioned values.

In several embodiments, the IN epinephrine pharmaceutical formulations include sodium metabisulfite present at a concentration of 0.1 mg/mL to 1.0 mg/mL, or any concentration range subsumed therein, including but not limited to, 0.1 mg/mL to 0.9 mg/mL, 0.1 mg/mL to 0.8 mg/mL, 0.1 mg/mL to 0.5 mg/mL, 0.2 mg/mL to 0.5 mg/mL, or 0.2 mg/mL to 0.4 mg/mL. In several embodiments, the IN epinephrine pharmaceutical formulations include sodium metabisulfite present at a concentration of equal to or less than about: 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, or 1.0 mg/mL, or ranges including and/or spanning the aforementioned values.

In several embodiments of the IN epinephrine pharmaceutical formulation, the antioxidant is sodium metabisulfite present in a concentration range of 0.5 mg/mL to 1.0 mg/mL. In several embodiments of the IN epinephrine pharmaceutical formulations, the antioxidant is sodium metabisulfite present at a concentration of equal to or less than about: 0.50 mg/mL, 0.55 mg/mL, 0.60 mg/mL, 0.65 mg/mL, 0.70 mg/mL, 0.75 mg/mL, 0.80 mg/mL, 0.85 mg/mL, 0.90 mg/mL, 0.95 mg/mL, or 1.0 mg/mL, or ranges including and/or spanning the aforementioned values.

In several embodiments, the IN epinephrine pharmaceutical formulation comprises the antioxidant at a molarity of equal to or less than about: 0.001 M, 0.002 M, 0.004 M, 0.005 M, 0.006 M, 0.007 M, 0.0080 M, 0.010 M, or ranges including and/or spanning the aforementioned values. For example, in several embodiments, the IN epinephrine pharmaceutical formulation comprises the antioxidant at a molarity ranging from 0.001 M to 0.010 M, 0.002 M to 0.004 M, 0.001 M to 0.005 M. pH Adjustor

In several embodiments of the IN epinephrine pharmaceutical formulations, the formulations include water. In other embodiments of the IN epinephrine pharmaceutical formulations, other suitable solvents may be included, such as an alcohol solvent or other organic solvents, in addition to or instead of water. In several embodiments, the IN epinephrine pharmaceutical formulations include a pH adjustor, such as hydrochloric acid (HCl), sodium hydroxide (NaOH), acetic acid, ascorbic acid, sulphuric acid, tartaric acid, or a combination thereof. In several embodiments of the IN epinephrine pharmaceutical formulations, the pH adjustor includes 10% (w/v) HCl and as needed, NaOH.

Thus, in several embodiments, the IN epinephrine pharmaceutical formulations include 1.0 mg/mL to 25.0 mg/mL of epinephrine, or a pharmaceutically acceptable salt thereof, 1.0 mg/mL to 15.0 mg/mL of a bile salt, 1.0 mg/mL to 8.0 mg/mL of a first buffer, 5.0 mg/mL to 10.0 mg/mL of a second buffer, 3.0 mg/mL to 8.0 mg/mL of a preservative, 1.0 mg/mL to 4.0 mg/mL of a tonicity agent, 0.01 mg/mL to 0.05 mg/mL of metal complexing agent, 0.1 mg/mL to 1.0 mg/mL of an antioxidant, and wherein the pharmaceutical formulation has a pH of 2.2 to 5.0 and is for intranasal (IN) delivery.

In several embodiments, the IN epinephrine pharmaceutical formulations include 1.0 mg/mL to 25.0 mg/mL of epinephrine, or a pharmaceutically acceptable salt thereof, 1.0 mg/mL to 15.0 mg/mL of a bile salt, wherein the bile salt is STC, 1.0 mg/mL to 8.0 mg/mL of citric acid, 5.0 mg/mL to 10.0 mg/mL of sodium citrate, 3.0 mg/mL to 8.0 mg/mL of chlorobutanol, 1.0 mg/mL to 4.0 mg/mL of sodium chloride, 0.01 mg/mL to 0.05 mg/mL of disodium edetate dihydrate, 0.1 mg/mL to 1.0 mg/mL of sodium metabisulfite, and wherein the pharmaceutical formulation has a pH of 2.2 to 5.0 and is for intranasal (IN) delivery.

In several embodiments, the IN epinephrine pharmaceutical formulations include 5.0 mg/mL to 13.0 mg/mL of epinephrine, or a pharmaceutically acceptable salt thereof, 5.0 mg/mL to 12.0 mg/mL of a bile salt, 1.0 mg/mL to 8.0 mg/mL of a first buffer, 5.0 mg/mL to 10.0 mg/mL of a second buffer, 3.0 mg/mL to 8.0 mg/mL of a preservative, 1.0 mg/mL to 4.0 mg/mL of a tonicity agent, 0.01 mg/mL to 0.05 mg/mL of metal complexing agent, 0.1 mg/mL to 1.0 mg/mL of an antioxidant, and wherein the pharmaceutical formulation has a pH of 3.0 to 4.0 and is for intranasal (IN) delivery.

In several embodiments, the IN epinephrine pharmaceutical formulations include 5.0 mg/mL to 13.0 mg/mL of epinephrine, or a pharmaceutically acceptable salt thereof, 5.0 mg/mL to 12.0 mg/mL of a bile salt, wherein the bile salt is STC, 3.0 mg/mL to 5.0 mg/mL of citric acid, 6.0 mg/mL to 10.0 mg/mL of sodium citrate, 4.0 mg/mL to 7.0 mg/mL of chlorobutanol, 1.0 mg/mL to 5.0 mg/mL of sodium chloride, 0.01 mg/mL to 0.05 mg/mL of disodium edetate dihydrate, 0.1 mg/mL to 1.0 mg/mL of sodium metabisulfite, wherein the pharmaceutical formulation has a pH of 3.7 to 3.9, and wherein the pharmaceutical formulation is for IN delivery. In these embodiments of the IN epinephrine pharmaceutical formulations, the pH can be about 3.7, about 3.8, or about 3.9.

In several embodiments, the IN epinephrine pharmaceutical formulations include 7.0 mg/mL to 9.0 mg/mL of epinephrine, or a pharmaceutically acceptable salt thereof, 7.0 mg/mL to 9.0 mg/mL of STC, 3.0 mg/mL to 5.0 mg/mL of citric acid, 6.0 mg/mL to 10.0 mg/mL of sodium citrate, 4.0 mg/mL to 7.0 mg/mL of chlorobutanol, 1.0 mg/mL to 5.0 mg/mL of sodium chloride, 0.01 mg/mL to 0.05 mg/mL of disodium edetate dihydrate, 0.1 mg/mL to 1.0 mg/mL of sodium metabisulfite, wherein the pharmaceutical formulation has a pH of 3.7 to 3.9, and wherein the pharmaceutical formulation is for IN delivery. In these embodiments the IN epinephrine pharmaceutical formulations, the pH can be about 3.7, about 3.8, or about 3.9.

In several embodiments, the IN epinephrine pharmaceutical formulations include 6.0 mg/mL to 10.0 mg/mL of epinephrine, or a pharmaceutically acceptable salt thereof, 7.0 mg/mL to 9.0 mg/mL of STC, 3.0 mg/mL to 5.0 mg/mL of citric acid, 6.0 mg/mL to 10.0 mg/mL of sodium citrate, 4.0 mg/mL to 7.0 mg/mL of chlorobutanol, 1.0 mg/mL to 3.0 mg/mL of sodium chloride, 0.01 mg/mL to 0.05 mg/mL of disodium edetate dihydrate, 0.1 mg/mL to 0.5 mg/mL of sodium metabisulfite, wherein the pharmaceutical formulation has a pH of 3.7 to 3.9, and wherein the pharmaceutical formulation is configured for IN delivery. In several embodiments, the pH can be between about 3.7 and 3.9. In several embodiments, the IN epinephrine pharmaceutical formulations include 10.0 mg/mL to 14.0 mg/mL of epinephrine, or a pharmaceutically acceptable salt thereof, 8.0 mg/mL to 12.0 mg/mL of STC, 3.0 mg/mL to 5.0 mg/mL of citric acid, 6.0 mg/mL to 10.0 mg/mL of sodium citrate, 4.0 mg/mL to 7.0 mg/mL of chlorobutanol, 1.0 mg/mL to 3.0 mg/mL of sodium chloride, 0.01 mg/mL to 0.05 mg/mL of disodium edetate dihydrate, 0.1 mg/mL to 0.5 mg/mL of sodium metabisulfite, wherein the pharmaceutical formulation has a pH of 3.7 to 3.9, and wherein the pharmaceutical formulation is configured for IN delivery. In several embodiments, the pH can be between about 3.7 and 3.9.

In several embodiments, the IN epinephrine pharmaceutical formulations include 11.0 mg/mL to 13.0 mg/mL of epinephrine, or a pharmaceutically acceptable salt thereof, 9.0 mg/mL to 11.0 mg/mL of STC, 3.0 mg/mL to 5.0 mg/mL of citric acid, 6.0 mg/mL to 10.0 mg/mL of sodium citrate, 4.0 mg/mL to 7.0 mg/mL of chlorobutanol, 1.0 mg/mL to 3.0 mg/mL of sodium chloride, 0.01 mg/mL to 0.05 mg/mL of disodium edetate dihydrate, 0.1 mg/mL to 0.5 mg/mL of sodium metabisulfite, wherein the pharmaceutical formulation has a pH of 3.7 to 3.9, and wherein the pharmaceutical formulation is configured for IN delivery. In several embodiments, the pH can be between about 3.7 and 3.9.

### IN Delivery Using a Nasal Spray

In several embodiments, the disclosed IN pharmaceutical formulations can be administered by IN delivery using a nasal spray. Nasal sprays facilitate IN delivery of epinephrine pharmaceutical formulations to one or more nostrils of a human patient. In several embodiments, the nasal spray has a spray pump for discharging a dose volume of the pharmaceutical formulation in a single spray to a single nostril, or in two or more sprays to one or more nostrils. In several embodiments, the dose volume of the IN epinephrine pharmaceutical formulation contains the dose amount of the epinephrine, or a pharmaceutically acceptable salt thereof.

In several embodiments, the nasal spray is a unit-dose nasal spray that administers a single dose volume of the pharmaceutical formulation in a single spray to a single nostril, or in two or more sprays to one or more nostrils, and such unit-dose nasal spray is disposed thereafter. In other embodiments, the nasal spray is a bi-dose nasal spray that can administer two dose volumes of the pharmaceutical formulation in two or more sprays to one or more nostrils, and such bi-dose nasal spray is disposed thereafter. In several embodiments, the unit-dose nasal spray or the bi-dose nasal spray is pre-primed to provide accurate dosing and ready to use capability. In still other embodiments, the nasal spray can administer three or more dose volumes of the pharmaceutical formulation.

In several embodiments, the dose volume of the IN epinephrine pharmaceutical formulation is from 0.01 mL to 0.30 mL. In several embodiments, the dose volume of the IN epinephrine pharmaceutical formulation is from 0.05 mL to 0.15 mL. In several embodiments, the dose volume of the IN epinephrine pharmaceutical formulation is about 0.10 mL. The dose volume is the volume that contains the dose amount of the API. In several embodiments, the dose volume is equal to or less than about: 0.05 mL, 0.075 mL, 0.1 mL, 0.125 mL, 0.15 mL, 0.2 mL, or ranges including and/or spanning the aforementioned values.

In several embodiments, the dose amount of the epinephrine, or the pharmaceutically acceptable salt thereof, is in a range of 0.1 mg to 2.5 mg, or any amount range subsumed therein, including but not limited to, 0.5 mg to 1.5 mg, 0.75 mg to 1.25 mg, 0.8 mg to 1.2 mg, 0.9 mg to 1.1 mg, 0.95 mg to 1.05 mg. In several embodiments, the dose amount of the epinephrine, or the pharmaceutically acceptable salt thereof, is in an amount of equal to or less than about: 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, or ranges including and/or spanning the aforementioned values. To illustrate, if the IN epinephrine pharmaceutical formulation has an epinephrine concentration of about 8 mg/mL and the dose volume is about 0.1 mL, then the dose amount of the epinephrine, or a pharmaceutically acceptable salt thereof, would be about 0.8 mg. In several embodiments, a single spray from the dispensing device provides a dose of epinephrine, or the pharmaceutically acceptable salt thereof, in a range of 0.1 mg to 2.5 mg, or any amount range subsumed therein, including but not limited to, 0.5 mg to 1.5 mg, 0.75 mg to 1.25 mg, 0.8 mg to 1.2 mg, 0.9 mg to 1.1 mg, 0.95 mg to 1.05 mg. In several embodiments, a single spray from the dispensing device provides a dose of epinephrine, or the pharmaceutically acceptable salt thereof, in an amount of equal to or less than about: 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, or ranges including and/or spanning the aforementioned values.

In several embodiments, the dose amount of the bile acid, or the salt thereof (such as STC), is in a range of 0.1 mg to 1.5 mg, or any amount range subsumed therein, including but not limited to, 0.5 mg to 1.1 mg, 0.6 mg to 1.3 mg, 0.7 mg to 1.2 mg, 0.75 mg to 0.95 mg, 0.75 mg to 0.85 mg, 0.7 mg to 0.9 mg, or 0.7 mg to 0.8 mg. In several embodiments, the dose amount of the bile salt, such as STC, is in an amount of equal to or less than about: 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, 1.5 mg, or ranges including and/or spanning the aforementioned values. To illustrate, if the IN epinephrine pharmaceutical formulation has an STC concentration of about 8 mg/mL and the dose volume is about 0.1 mL, then the dose amount of the STC would be about 0.8 mg. In several embodiments, a single spray from the dispensing device provides a dose of bile acid, or the salt thereof (such as STC) in a range of 0.1 mg to 2.5 mg, or any amount range subsumed therein, including but not limited to, 0.5 mg to 1.5 mg, 0.75 mg to 1.25 mg, 0.8 mg to 1.2 mg, 0.9 mg to 1.1 mg, 0.95 mg to 1.05 mg. In several embodiments, a single spray from the dispensing device provides a dose of bile acid, or the salt thereof (such as STC) in an amount of equal to or less than about: 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, 1.5 mg, or ranges including and/or spanning the aforementioned values.

Thus, in some embodiments, the IN epinephrine pharmaceutical formulation comprises epinephrine, or a pharmaceutically acceptable salt thereof, present in a dose amount of 0.1 mg to 2.5 mg, an absorption enhancer comprising a bile acid, or a salt thereof, present in a dose amount of 0.5 mg to 1.3 mg, wherein the pharmaceutical formulation has a dose volume of 0.05 mL to 0.25 mL, the pharmaceutical formulation has a pH of 2.2 to 5.0, and the pharmaceutical formulation is for IN delivery.

In several embodiments, the IN epinephrine pharmaceutical formulation comprises epinephrine, or a pharmaceutically acceptable salt thereof, present in a dose amount of 0.1 mg to 2.5 mg, an absorption enhancer comprising a bile acid, or a salt thereof, present in a dose amount of 0.5 mg to 1.3 mg, the bile salt is STC, wherein the pharmaceutical formulation has a dose volume of about 0.10 mL, the pharmaceutical formulation has a pH of 2.2 to 5.0, and the pharmaceutical formulation is for IN delivery.

In several embodiments, the pharmaceutical formulations further comprise the epinephrine, or the pharmaceutically acceptable salt thereof, present in a dose amount of 0.1 mg to 2.5 mg, or any amount range subsumed therein, including but not limited to, 0.1 mg to 4.5 mg, 0.1 mg to 4.25 mg, 0.1 mg to 4.0 mg, 0.1 mg to 3.5 mg, 0.1 mg to 3.25 mg, 0.1 mg to 3.0 mg, 0.1 mg to 2.75 mg, 0.1 mg to 2.5 mg, 0.1 mg to 2.25 mg, 0.1 mg to 2.0 mg, 0.1 mg to 1.75 mg, 0.1 mg to 1.5 mg, 0.1 mg to 1.25 mg, 0.1 mg to 1.0 mg, 0.1 mg to 0.75 mg, 0.1 mg to 0.5 mg, 0.1 mg to 0.25 mg, 0.25 mg to 2.5 mg, 0.25 mg to 2.25 mg, 0.25 mg to 2.0 mg, 0.25 mg to 1.75 mg, 0.25 mg to 1.5 mg, 0.25 mg to 1.25 mg, 0.25 mg to 1.0 mg, 0.25 mg to 0.75 mg, 0.25 mg to 0.5 mg, 0.5 mg to 2.5 mg, 0.5 mg to 2.25 mg, 0.5 mg to 2.0 mg, 0.5 mg to 1.75 mg, 0.5 mg to 1.5 mg, 0.5 mg to 1.3 mg, 0.5 mg to 1.25 mg, 0.5 mg to 1.0 mg, 0.5 mg to 0.75 mg, 0.75 mg to 2.5 mg, 0.75 mg to 2.25 mg, 0.75 mg to 2.0 mg, 0.75 mg to 1.75 mg, 0.75 mg to 1.5 mg, 0.75 mg to 1.3 mg, 0.75 mg to 1.25 mg, 0.75 mg to 1.0 mg, 1.0 mg to 2.0 mg.

In several embodiments, the pharmaceutical formulations further comprise the epinephrine, or the pharmaceutically acceptable salt thereof, present in a dose amount of equal to or less than about: 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg or ranges including and/or spanning the aforementioned values.

In several embodiments of the IN epinephrine pharmaceutical formulations, the epinephrine, or the pharmaceutically acceptable salt thereof, is present in a dose amount of 0.1 mg to 2.5 mg, or any amount range subsumed therein, including but not limited to, 0.5 mg to 1.5 mg, 0.5 mg to 1.3 mg, 0.7 mg to 0.9 mg, 0.75 mg to 1.25 mg, 0.8 mg to 1.2 mg, 0.9 mg to 1.1 mg, 0.95 mg to 1.05 mg. In several embodiments, the pharmaceutical formulation comprises the epinephrine, or the pharmaceutically acceptable salt thereof, in a dose amount of equal to or less than about: 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, or ranges including and/or spanning the aforementioned values.

In several embodiments of the IN epinephrine pharmaceutical formulation, the epinephrine, or the pharmaceutically acceptable salt thereof, is present at in a dose amount of 0.5 mg to 1.30 mg. In several embodiments of the IN epinephrine pharmaceutical formulation, the epinephrine, or the pharmaceutically acceptable salt thereof, is present at a dose amount of equal to or less than about: 0.50 mg, 0.55 mg, 0.60 mg, 0.70 mg, 0.75 mg, 0.80 mg, 0.85 mg, 0.90 mg, 0.95 mg, 1.00 mg, 1.05 mg, 1.10 mg, 1.15 mg, 1.20 mg, 1.25 mg, 1.30 mg, 1.50 mg, or ranges including and/or spanning the aforementioned values.

### Method of Providing A Rapid Absorption of Epinephrine Using IN Epinephrine Formulations For Treatments or Indications

The disclosed IN epinephrine pharmaceutical formulations can provide a rapid delivery of epinephrine into the bloodstream of a human patient by IN delivery, comparable to 1 mg/mL epinephrine IM auto-injectors. Additional embodiments disclose IN epinephrine pharmaceutical formulations that enhance absorption of epinephrine when compared to IN epinephrine pharmaceutical formulations without an enhancer. Surprisingly, as disclosed elsewhere herein, the compositions disclosed herein can provide delivery that is more rapid that other delivery systems (e.g., has a lower tₘₐₓ, higher AUC_{0-t*}, AUC₀₋₁₀ₘᵢₙ, AUC₀₋₃₀ₘᵢₙ, etc.) than other delivery systems, including IM or other IN compositions. As described herein, a bile salt, such as STC, can enhance the absorption of the epinephrine via the nasal mucosa, and into the bloodstream. The rapid delivery is a desired feature because of disclosed IN epinephrine formulations potential use as an emergency treatment.

Accordingly, disclosed are methods of providing a rapid delivery of epinephrine to a human patient, the method comprising the step of administrating a dose amount of epinephrine from any of the disclosed IN epinephrine pharmaceutical formulations to at least one nostril of a human patient to treat a condition, wherein the administrating is by intranasal (IN) delivery using a nasal spray, and wherein post-administration of the pharmaceutical formulation by IN delivery, a Cₘₐₓ of 5 ng/mL to 15 ng/mL and a tₘₐₓ of less than 15 minutes are achieved.

In several embodiments, as disclosed herein, methods of treating a condition are provided. In several embodiments, the method comprises identifying a patient (e.g., a human patient in need of treatment). In several embodiments, the patient in need of treatment is a patient suffering from a condition or at risk of suffering from a condition as disclosed elsewhere herein. In several embodiments, the method comprises administering a dose of a formulation as described herein to the patient. In several embodiments, the dose is provided as one or more sprays from a dispensing device. In several embodiments, the dose is delivered to the nostril of the patient (or both nostrils). In several embodiments, the patient is treated after receiving the dose.

In several embodiments, the condition is a type-I hypersensitivity reaction (e.g., systemic allergic reaction), an acute asthmatic attack, cardiac arrest, Stokes-Adams Syndrome, or a combination of the foregoing In several embodiments, the condition is an allergic reaction, such as Type 1 allergic reactions. In several embodiments, the condition is a type-I hypersensitivity reaction (systemic allergic reaction), an acute asthmatic attack, cardiac arrest, Stokes-Adams Syndrome, or a combination of the foregoing. Anaphylaxis is an example of a Type 1 allergic reaction. In other embodiments, the condition is hypotension associated with septic shock, or for increasing mean arterial blood pressure in a patient with hypotension associated with septic shock. In several embodiments, the type-I hypersensitivity reaction is selected from allergic asthma, allergic conjunctivitis, allergic rhinitis, anaphylaxis, angioedema, urticaria, eosinophilia, drug allergy, and food allergy. In several embodiments, the condition is an emergency condition. In several embodiments, the condition includes bronchospasm, sensitivity reactions, cardiopulmonary resuscitation, cardiac arrhythmias, local vasoconstriction, premature labor, hypoglycemia, gastrointestinal hemorrhage, renal hemorrhage, bleeding, or mydriasis during intraocular surgery. In several embodiments, the pharmaceutical formulation is used in a method of increasing mean arterial blood pressure in patients with hypotension associated with septic shock, to relieve respiratory distress due to bronchospasm, to provide rapid relief of hypersensitivity reactions to drugs and other allergens, to prolong the action of infiltration anesthetics, and/or combinations thereof.

In several embodiments, the disclosed IN pharmaceutical formulations can achieve an AUC_{0-10 min}, AUC₀₋₃₀ₘᵢₙ, AUC₀₋₁₈₀ₘᵢₙ, AUC_{0-Xmin}, Cₘₐₓ, tₘₐₓ, and bioavailability, including relative bioavailability (RBA), similar to that of IM epinephrine auto-injectors (e.g. 1 mg/mL IM epinephrine injector) at a similar rate, or a similar time period, as that of the IM epinephrine auto-injector. In other embodiments, the disclosed IN pharmaceutical formulations can achieve an AUC_{0-10 min}, AUC₀₋₃₀ₘᵢₙ, AUC₀₋₁₈₀ₘᵢₙ, Cₘₐₓ, and bioavailability similar to that of an IM epinephrine auto-injectors (e.g. 1 mg/mL IM epinephrine auto-injector) at a faster rate, or a shorter time period, compared to that of the IM epinephrine auto-injector. An example of a 1 mg/mL IM epinephrine auto-injector is an EpiPen® (0.3 mg epinephrine).

In several embodiments, an IN composition as disclosed herein achieves a Cₘₐₓ of greater than or equal to about: 100 pg/mL, 200 pg/mL, 300 pg/mL, 350 pg/mL, 400 pg/mL, 450 pg/mL, 500 pg/mL, 550 pg/mL, 600 pg/mL, 650 pg/mL, or ranges including and/or spanning the aforementioned values. For example, in several embodiments, an IN composition as disclosed herein achieves a Cₘₐₓ ranging from 100 pg/mL to 650 pg/mL, 300 pg/mL to 650 pg/mL, 350 pg/mL to 600 pg/mL, 300 pg/mL to 650 pg/mL, 400 pg/mL to 650 pg/mL, 450 pg/mL to 600 pg/mL, etc. In several embodiments, the Cₘₐₓ is measured as the geometric mean of a representative patient population. In several embodiments, the Cₘₐₓ is measured as the arithmetic mean of a representative patient population. In several embodiments, the Cₘₐₓ for the IN composition differs from the Cₘₐₓ for an IM formulation epinephrine by less than or equal to about: 40%, 30%, 20%, 10%, 5%, or ranges including and/or spanning the aforementioned values.

In several embodiments, an IN composition as disclosed herein achieves a tₘₐₓ (in minutes) of less than or equal to about: 5, 6, 7, 8, 9, 10, 11, 12, 13, 14, 15, 17.5, 20, 25, or ranges including and/or spanning the aforementioned values. For example, in several embodiments, an IN composition as disclosed herein achieves a tₘₐₓ (in minutes) ranging from 5 to 15, 7 to 10, 6 to 12, 5 to 10, 5 to 20, etc. In several embodiments, the tₘₐₓ is measured as the geometric mean of a representative patient population. In several embodiments, the tₘₐₓ is measured as the arithmetic mean of a representative patient population. In several embodiments, the tₘₐₓ for the IN composition differs from the tₘₐₓ for an IM formulation epinephrine by less than or equal to about: 40%, 30%, 20%, 10%, 5%, or ranges including and/or spanning the aforementioned values.

In several embodiments, an IN composition as disclosed herein achieves a AUC_{0-t*} of greater than or equal to about: 10 pg/mL*hr, 15 pg/mL*hr, 20 pg/mL*hr, 25 pg/mL*hr, 26 pg/mL*hr, 27 pg/mL*hr, 28 pg/mL*hr, 29 pg/mL*hr, 30 pg/mL*hr, 32 pg/mL*hr, 35 pg/mL*hr, 40 pg/mL*hr, 45 pg/mL*hr, 50 pg/mL*hr, or ranges including and/or spanning the aforementioned values. For example, in several embodiments, an IN composition as disclosed herein achieves a AUC_{0-t*} ranging from 10 pg/mL*hr to 35 pg/mL*hr, 15 pg/mL*hr to 30 pg/mL*hr, 25 pg/mL*hr to 35 pg/mL*hr, 30 pg/mL*hr to 35 pg/mL*hr, 28 pg/mL*hr to 35 pg/mL*hr, 20 pg/mL*hr to 40 pg/mL*hr, etc. In several embodiments, the AUC_{0-t*} is measured as the geometric mean of a representative patient population. In several embodiments, the AUC_{0-t*} is measured as the arithmetic mean of a representative patient population. In several embodiments, the AUC_{0-t*} for the IN composition differs from the AUC_{0-t*} for an IM formulation epinephrine by less than or equal to about: 40%, 30%, 20%, 10%, 5%, or ranges including and/or spanning the aforementioned values.

In several embodiments, an IN composition as disclosed herein achieves a AUC_{0-10 min} of greater than or equal to about: 10 pg/mL*hr, 15 pg/mL*hr, 20 pg/mL*hr, 25 pg/mL*hr, 30 pg/mL*hr, 35 pg/mL*hr, 40 pg/mL*hr, 45 pg/mL*hr, 50 pg/mL*hr, 55 pg/mL*hr, 65 pg/mL*hr, or ranges including and/or spanning the aforementioned values. For example, in several embodiments, an IN composition as disclosed herein achieves a AUC_{0-10 min} ranging from 20 pg/mL*hr to 50 pg/mL*hr, 10 pg/mL*hr to 60 pg/mL*hr, 25 pg/mL*hr to 55 pg/mL*hr, 40 pg/mL*hr to 50 pg/mL*hr, 45 pg/mL*hr to 60 pg/mL*hr, 20 pg/mL*hr to 60 pg/mL*hr, etc. In several embodiments, the AUC_{0-10 min} is measured as the geometric mean of a representative patient population. In several embodiments, the AUC_{0-10 min} is measured as the arithmetic mean of a representative patient population. In several embodiments, the AUC_{0-10 min} for the IN composition differs from the AUC_{0-10 min} for an IM formulation epinephrine by less than or equal to about: 40%, 30%, 20%, 10%, 5%, or ranges including and/or spanning the aforementioned values.

In several embodiments, an IN composition as disclosed herein achieves a AUC₀₋₃₀ₘᵢₙ of greater than or equal to about: 30 pg/mL*hr, 40 pg/mL*hr, 50 pg/mL*hr, 60 pg/mL*hr, 70 pg/mL*hr, 80 pg/mL*hr, 90 pg/mL*hr, 100 pg/mL*hr, 110 pg/mL*hr, 120 pg/mL*hr, 130 pg/mL*hr, 140 pg/mL*hr, 150 pg/mL*hr, 160 pg/mL*hr, 170 pg/mL*hr, or ranges including and/or spanning the aforementioned values. For example, in several embodiments, an IN composition as disclosed herein achieves a AUC₀₋₃₀ₘᵢₙ ranging from 90 pg/mL*hr to 140 pg/mL*hr, 100 pg/mL*hr to 160 pg/mL*hr, 70 pg/mL*hr to 140 pg/mL*hr, 120 pg/mL*hr to 140 pg/mL*hr, 60 pg/mL*hr to 160 pg/mL*hr, 130 pg/mL*hr to 140 pg/mL*hr, etc. In several embodiments, the AUC₀₋₃₀ₘᵢₙ is measured as the geometric mean of a representative patient population. In several embodiments, the AUC₀₋₃₀ₘᵢₙ is measured as the arithmetic mean of a representative patient population.

In several embodiments, an IN composition as disclosed herein achieves a AUC_{0-6 hrs} of greater than or equal to about: 100 pg/mL*hr, 200 pg/mL*hr, 250 pg/mL*hr, 300 pg/mL*hr, 325 pg/mL*hr, 350 pg/mL*hr, 375 pg/mL*hr, 400 pg/mL*hr, 425 pg/mL*hr, 450 pg/mL*hr, 475 pg/mL*hr, 500 pg/mL*hr, 550 pg/mL*hr, or ranges including and/or spanning the aforementioned values. For example, in several embodiments, an IN composition as disclosed herein achieves a AUC_{0-6 hrs} ranging from 300 pg/mL*hr to 500 pg/mL*hr, 250 pg/mL*hr to 350 pg/mL*hr, 300 pg/mL*hr to 450 pg/mL*hr, 250 pg/mL*hr to 500 pg/mL*hr, 100 pg/mL*hr to 550 pg/mL*hr, 425 pg/mL*hr to 475 pg/mL*hr, etc. In several embodiments, the AUC_{0-6 hrs} is measured as the geometric mean of a representative patient population. In several embodiments, the AUC₀₋₆ₕᵣ is measured as the arithmetic mean of a representative patient population.

In several embodiments, an IN composition as disclosed herein achieves a AUC_{0-∞} of greater than or equal to about: 100 pg/mL*hr, 200 pg/mL*hr, 250 pg/mL*hr, 300 pg/mL*hr, 325 pg/mL*hr, 350 pg/mL*hr, 375 pg/mL*hr, 400 pg/mL*hr, 425 pg/mL*hr, 450 pg/mL*hr, 475 pg/mL*hr, 500 pg/mL*hr, 550 pg/mL*hr, 600 pg/mL*hr, or ranges including and/or spanning the aforementioned values. For example, in several embodiments, an IN composition as disclosed herein achieves a AUC_{0-∞} ranging from 300 pg/mL*hr to 550 pg/mL*hr, 250 pg/mL*hr to 600 pg/mL*hr, 350 pg/mL*hr to 550 pg/mL*hr, 500 pg/mL*hr to 550 pg/mL*hr, 100 pg/mL*hr to 600 pg/mL*hr, 375 pg/mL*hr to 550 pg/mL*hr, etc. In several embodiments, the AUC_{0-∞} is measured as the geometric mean of a representative patient population. In several embodiments, the AUC_{0-∞} is measured as the arithmetic mean of a representative patient population.

In several embodiments, the IN pharmaceutical formulations can achieve a Cₘₐₓ in a range of 5 ng/mL to 15 ng/mL, or any range subsumed therein, including but not limited to 5 ng/mL to 10 ng/mL, 7 ng/mL to 14 ng/mL, 8 ng/mL to 13 ng/mL, 10 ng/mL to 15 ng/mL, or 11 ng/mL to 15 ng/mL. In other embodiments, the Cₘₐₓ is about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, or about 15 ng/mL. By comparison, an IM epinephrine auto-injector, such as a 1 mg/mL IM epinephrine auto-injector can achieve a Cₘₐₓ of 12.1 ng/mL.

In several embodiments, the IN pharmaceutical formulations can achieve a tₘₐₓ in less than 25 minutes (or any range subsumed therein), including, but not limited to, less than 24, 23, 22, 21, 20, 19, 18, 17, 16, 15, 14, 13, 12, 11, 10, 9, 8, 7, 6, 5, 4, 3, 2, or 1 minute(s).

In several embodiments, the IN pharmaceutical formulations can achieve a 100% relative bioavailability of epinephrine with respect to a 1 mg/mL IM epinephrine auto-injector. In other embodiments, the IN pharmaceutical formulations can achieve a relative bioavailability of epinephrine of 75% to 125% (or any range subsumed therein) with respect to a 1 mg/mL IM epinephrine auto-injector.

In several embodiments, the IN pharmaceutical formulations can achieve an AUC_{0-10 min} in a range of 50 (ng*min)/mL to 80 (ng*min)/mL, or any range subsumed therein, including, but not limited to, 55 (ng*min)/mL to 65 (ng*min)/mL, 60 (ng*min)/mL to 70 (ng*min)/mL, or 65 (ng*min)/mL to 75 (ng*min)/mL. In other embodiments, the IN pharmaceutical formulations can achieve an AUC_{0-10 min} of at least 50 (ng*min)/mL, 55 (ng*min)/mL, 60 (ng*min)/mL, 65 (ng*min)/mL, 70 (ng*min)/mL, 75 (ng*min)/mL, or 80 (ng*min)/mL. By comparison, an IM epinephrine auto-injector, such as a 1 mg/mL IM epinephrine auto-injector, can achieve an AUC_{0-10 min} of 64 (ng*min)/mL.

In several embodiments, the IN pharmaceutical formulations can achieve an AUC₀₋₃₀ₘᵢₙ in a range of 100 (ng*min)/mL to 170 (ng*min)/mL or any range subsumed therein, including, but not limited to, 115 (ng*min)/mL to 135 (ng*min)/mL, 115 (ng*min)/mL to 130 (ng*min)/mL, or 120 (ng*min)/mL to 130 (ng*min)/mL. In other embodiments, the IN pharmaceutical formulations can achieve an AUC₀₋₃₀ₘᵢₙ of at least 110 (ng*min)/mL, 115 (ng*min)/mL, 120 (ng*min)/mL, 125 (ng*min)/mL, 130 (ng*min)/mL, 135 (ng*min)/mL, or 140 (ng*min)/mL. By comparison, an IM epinephrine auto-injector, such as a 1 mg/mL IM epinephrine auto-injector, can achieve an AUC₀₋₃₀ₘᵢₙ s of 133 (ng*min)/mL.

In several embodiments, the IN pharmaceutical formulations can achieve an AUC₀₋₁₈₀ₘᵢₙ in a range of 150 (ng*min)/mL to 300 (ng*min)/mL or any range subsumed therein, including, but not limited to, 150 (ng*min)/mL to 275 (ng*min)/mL, 150 (ng*min)/mL to 250 (ng*min)/mL, 150 (ng*min)/mL to 225 (ng*min)/mL, 150 (ng*min)/mL to 200 (ng*min)/mL, 175 (ng*min)/mL to 275 (ng*min)/mL, 175 (ng*min)/mL to 250 (ng*min)/mL, 175 (ng*min)/mL to 225 (ng*min)/mL, 175 (ng*min)/mL to 200 (ng*min)/mL, 200 (ng*min)/mL to 300 (ng*min)/mL, 200 (ng*min)/mL to 275 (ng*min)/mL, or 200 (ng*min)/mL to 250 (ng*min)/mL. In other embodiments, the IN pharmaceutical formulations can achieve an AUC₀₋₁₈₀ₘᵢₙ of at least 150 (ng*min)/mL, 160 (ng*min)/mL, 170 (ng*min)/mL, 180 (ng*min)/mL, 190 (ng*min)/mL, 200 (ng*min)/mL, 210 (ng*min)/mL, 220 (ng*min)/mL, 230 (ng*min)/mL, 240 (ng*min)/mL, 250 (ng*min)/mL, 260 (ng*min)/mL, 270 (ng*min)/mL, 280 (ng*min)/mL, 290 (ng*min)/mL, or 300 (ng*min)/mL.

In several embodiments, if the bile acid, or the salt thereof, causes decreased cilia in a respiratory epithelium of the human subject, then such decrease is substantially reversed within 7 days, including but not limited to, within 6 days, within 5 days, within 4 days, within 3 days, within 2 days, or within 1 day. In other embodiments, if the bile acid, or the salt thereof, causes hyperplasia of a respiratory epithelium of the human subject, then such hyperplasia is substantially reversed within 7 days, including but not limited to, within 6 days, within 5 days, within 4 days, within 3 days, within 2 days, or within 1 day. In still other embodiments, if the bile acid, or the salt thereof, causes decreased cilia and hyperplasia of a respiratory epithelium of the human subject, then such decreased cilia and hyperplasia are substantially reversed within 7 days, including but not limited to, within 6 days, within 5 days, within 4 days, within 3 days, within 2 days, or within 1 day. In further embodiments, if the bile acid, or the salt thereof, causes any change to a nasal mucosa of the human subject, then such change is substantially reversed within 7 days, including but not limited to, within 6 days, within 5 days, within 4 days, within 3 days, within 2 days, or within 1 day.

In several embodiments, surprisingly, despite having one or more of a higher total dose, Cₘₐₓ, AUC_{0-10 min}, AUC₀₋₃₀ₘᵢₙ, AUC_{0-180 min}, AUC₀₋₆ₕᵣ, and/or AUC_{0-∞} than a comparator IM administration of epinephrine (e.g., provided from an EpiPen®), a dose of the IN epinephrine formulation disclosed herein causes a number of milder side effects (e.g., adverse events) than the IM dose. In several embodiments, surprisingly, despite having a lower tₘₐₓ than a comparator IM administration of epinephrine (e.g., provided from an EpiPen®), a dose of the IN epinephrine formulation disclosed herein causes a number of milder side effects (e.g., adverse events) than the IM dose. In several embodiments, the milder side effects are one or more of nausea, vomiting, tachycardia, bradycardia, tremor, diastolic hypertension, hypotension, tachypnea, or combinations of the foregoing. In several embodiments, the incidence for of adverse events, as disclosed herein, for IN composition is less than that for an IM formulation epinephrine by equal to or at least about: 40%, 30%, 20%, 10%, 5%, or ranges including and/or spanning the aforementioned values.

Surprisingly, it has been found that several embodiments of the IN formulations disclosed herein have less incidences of mucosal edema, rhinorrhea, nasal discharge, and/or nasal discomfort at an increased concentration (e.g., over 8 mg/mL) of the enhancing agent. In several embodiments, the incidence for of mucosal edema, rhinorrhea, nasal discharge, and/or nasal discomfort is reduced by equal to or at least about: 40%, 30%, 20%, 10%, 5%, or ranges including and/or spanning the aforementioned values. In several embodiments, surprisingly, at an increased concentration (e.g., over 8 mg/mL) of the enhancing agent, the incidents of severe events and/or grade 3 occur based on a Nasal and Oropharyngeal Mucosa Exam (NOME) scale does not increase. In several embodiments, surprisingly, at an increased concentration (e.g., over 8 mg/mL) of the enhancing agent, the incidents of severe events and/or grade 3 occur based on a Self-Reported Nasal Symptoms (SRNS) scale does not increase. In several embodiments, surprisingly, a population of subjects experiences higher rates of normosmia 6 hours after an IN dose as measured by the University of Pennsylvania Smell Identification Test (UPSIT).

### Devices

Several embodiments provided herein provide dispensing devices for administering the compositions disclosed herein. In several embodiments, the IN pharmaceutical compositions may, if desired, be presented in a dispenser device. The dispenser device may be accompanied by instructions for administration. The dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include epinephrine, a bile acid, and/or salts may be placed in an appropriate container and/or dispending device, and labeled for treatment of an indicated condition.

In several embodiments, the dispensing device comprises a vessel. The term “vessel” is used herein in accordance with its ordinary meaning in the art and includes any structure which is capable of holding an epinephrine IN composition and being in fluid communication with a dispensing component. A dispensing component may be any feature which is capable of being in fluid communication with a vessel and dispensing an epinephrine IN composition from the vessel to a nasal cavity of a person. Examples may include, but are not limited to, valves, lumens, orifices, etc. These dispensing components may be shaped to fit a nostril of a human patient. These devices may be further packaged in a film or other sealing material that may be configured to easily be removed prior to use of the device.

In several embodiments, the vessel may comprise a cylinder and may contain a liquid epinephrine IN composition. The vessel may be connected to a dispensing component. In several embodiments, the dispensing device comprises an actuator (a trigger, button, etc.) that may be actuated to dispense the composition. In several embodiments, the dispensing device is configured to distribute a spray. In several embodiments, the vessel and/or dispensing component can be in fluid communication with a spray-producing component. In some embodiments, the dispensing component is a spray-producing component. The spray-producing component may be capable of dispensing the epinephrine IN composition in a form comprising a spray.

The device may further comprise a propellant gas, which may fill the headspace above the liquid epinephrine IN composition and be dissolved in the liquid so that when the spray component is opened, the pressure difference may cause spray to be dispensed. The propellant gas dissolved in the liquid may provide foam or spray as the dissolved gas expands under the reduced pressure. Inert gases, such as nitrogen, carbon dioxide, and fluorocarbons, may be useful as propellants.

### Examples

### Example 1-Bile Salt Enhances Intranasal (IN) Absorption of API

Example 1 presents an animal study that demonstrates that bile salts can enhance the IN absorption of an API. In Example 1, STC (hydrate) is the exemplary bile salt used, and epinephrine is the exemplary API used. Other suitable bile salts may also be utilized. The Example 1 pharmaceutical formulations tested are detailed in Tables 1.1-1.2. For brevity throughout this disclosure, “Epi” or “epi” refers to epinephrine, and “Conc.” refers to concentration.

TABLE 1.1
Formulations Tested (Excipients Not Shown)

| | | | | Bile | |
|---|---|---|---|---|---|
| Delivery | Formulation | Arm | Epi Conc. | Salt Conc. | Volume |
| Route | No. | Code | (mg/mL) | (STC) (mg/mL) | (μL) |
| IM | 0 | M | 1 | 0 | 25 |
| IN | 1 | T2a | 2 | 4 | 25 |
| | 2 | T2b | 2 | 6 | 25 |
| | 3 | T2c | 2 | 8 | 25 |
| | 4 | T2d | 2 | 10 | 25 |
| | 5 | T1 | 4 | 0 | 25 |
| | 6 | T3a | 4 | 4 | 25 |
| | 7 | T3b | 4 | 6 | 25 |
| | 8 | T3c | 4 | 8 | 25 |
| | 9 | T3d | 4 | 10 | 12.5 |
| | 10 | T4a | 8 | 4 | 25 |
| | 11 | T4b | 8 | 6 | 25 |
| | 12 | T4c | 8 | 8 | 12.5 |
| | 13 | T5 | 10 | 6 | 12.5 |

TABLE 1.2
Dose of Formulations Tested (Excipients Not Shown)

| | | Mean | | | | Dose of | |
|---|---|---|---|---|---|---|---|
| | | Body | | | | Bile Salt | Number |
| Formulation | Arm | Weight | Dose of Epi | | | (STC) | of rats |
| No. | Code | (kg) | d, mg | mg/kg | MRDH | (kg/mg) | (n) |
| 0 | M | 0.30 | 0.025 | 0.08 | 8 | 0.00 | 20 |
| 1 | T2a | 0.26 | 0.05 | 0.19 | 19 | 0.39 | 20 |
| 2 | T2b | 0.27 | 0.05 | 0.19 | 19 | 0.56 | 20 |
| 3 | T2c | 0.30 | 0.05 | 0.17 | 17 | 0.67 | 20 |
| 4 | T2d | 0.31 | 0.05 | 0.16 | 16 | 0.81 | 20 |
| 5 | T1 | 0.23 | 0.1 | 0.43 | 43 | 0.00 | 19 |
| 6 | T3a | 0.24 | 0.1 | 0.41 | 41 | 0.41 | 20 |
| 7 | T3b | 0.30 | 0.1 | 0.33 | 33 | 0.49 | 20 |
| 8 | T3c | 0.28 | 0.1 | 0.36 | 36 | 0.73 | 20 |
| 9 | T3d | 0.30 | 0.05 | 0.17 | 17 | 0.42 | 20 |
| 10 | T4a | 0.33 | 0.2 | 0.60 | 60 | 0.30 | 20 |
| 11 | T4b | 0.34 | 0.2 | 0.60 | 60 | 0.45 | 15 |
| 12 | T4c | 0.36 | 0.1 | 0.28 | 28 | 0.28 | 19 |
| 13 | T5 | 0.36 | 0.1 | 0.28 | 28 | 0.21 | 20 |

The excipients for the IN formulations in Table 1.1 include about 4 mg/mL citric acid (monohydrate), about 8 mg/mL sodium citrate (dihydrate), about 5.5 mg/mL chlorobutanol (hemihydrate), about 2-3 mg/mL sodium chloride, about 0.02 mg/mL EDTA (dihydrate), about 0.3 mg/mL sodium metabisulfite, and water for injection (q.s.). In addition, hydrochloric acid and sodium hydroxide may be added, as needed, to adjust the pH of the formulations to about 3.8.

In Table 1.2, MRDH is the Maximum Relative Dose for Human for EpiPen® 0.01 mg/kg. The dose for the EpiPen® is 0.3 mg for adults with a body weight of ≥30 kg. The relative dose for the EpiPen® will be 0.003-0.01 mg/kg if a body weight of 30 to 100 kg is used. Thus, the MRDH for the EpiPen® is 0.01 mg/kg. In this rat model PK study in Example 1, the rat IN dose is in the range of 0.16-0.6 mg/kg, i.e. 16 to 60 times of the MRDH for the EpiPen®, as also listed in Table 1.2. Also, the amount of STC delivered in this PK study in Example 1 is in the range of 0 to 0.81 mg/kg.

Additionally, an intramuscular (IM) injection formulation having 1 mg/mL epinephrine and no bile salt served as the reference control to the Table 1.1 formulations. This 1 mg/mL IM injection control has an Arm Code of “M” and is used as the baseline for determining relative bioavailability (RBA) for AUC₀₋₃₀ₘᵢₙ AUC₀₋₁₈₀ₘᵢₙ, Cₘₐₓ, and Bile Salt Enhancement Factor (EF), as will be shown in Table 1.3. More particularly, Example 1 assessed the dose-normalized relative bioavailability (DN-RBA), based on the ratio of the dose-normalized pharmacokinetic (PK) parameter by IN administration of the Table 1 formulations versus that of the 1 mg/mL IM injection control. The DN-RBA is defined as follows:

$${R}_{X} ( S ) = \frac{{X}_{IN} ( {d}_{IN ,} S ) \frac{1}{{d}_{IN}}}{{X}_{IM} ( {d}_{IM} ) \frac{1}{{d}_{IM}}}$$

where Rx is the DN-RBA for PK parameters X;
S is the concentration of Bile Salt (i.e. STC) used in the Table 1 formulations;
X are partial AUC, AUC₀₋₃₀ₘᵢₙ, and AUC₀₋₁₈₀ₘᵢₙ, or Cₘₐₓ;
d_{IM} and d_{IN} are doses delivered by IM and IN routes, respectively.

The Bile Salt EF_{0-30/0-180/Cmax} to the IN absorption of the API (i.e. epinephrine), based on the DN-RBA equation above, is defined as follows:

$$EF ( S ) = \frac{\overline{R} ( S )}{\overline{R} ( 0 )}$$

where ^{R̅}(S) is an average of DN-RBA for X (3 PK parameters: AUC₀₋₃₀ₘᵢₙ, AUC₀₋₁₈₀ₘᵢₙ, and Cₘₐₓ) by the IN route at a given Bile Salt concentration S.
   ^{R̅}(0) has the same definition at S=0, by the IN route.

Also shown in Table 1, for IN Formulations 1-8, 10-11, 25 μL of the respective formulation were administered by IN to the rats. For IN Formulations 9, 12, 13, only 12.5 μL of the respective formulation were administered by IN to the rats due to higher concentrations of bile salts and API. Also, the 1 mg/mL IM injection control were administered to 20 rats.

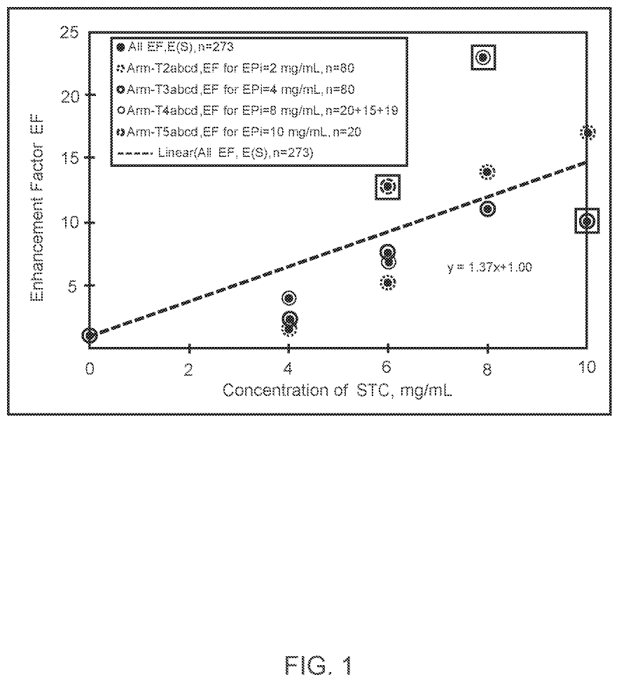

For the Bile Salt EF_{0-30/0-180/Cmax}, Formulation No. ^{R̅}(0) ved as because it has no STC. Table 1.3 and FIG. 1 show the results of the Bile Salt EF, which ranges from 1 to 23. Notably, Formulation No. 12 having 8 mg/mL epinephrine and 8 mg/mL bile salt (STC) had the highest Bile Salt EF at 23. Formulation No. 12 also exhibited a DN-RBA, IN v. IM of about 88% for AUC₀₋₃₀ₘᵢₙ, of about 61% for AUC₀₋₁₈₀ₘᵢₙ, and of about 106% for Cₘₐₓ, as well as a mean RBA v. IM of about 85%. Also shown in Table 1.3 is the equation B=d× EF, where d=dose of the API (in mg), and EF is the Bile Salt EF. B characterizes the net IN absorption of the API (epinephrine). Notably, Formulation No. 12 scored the highest net IN absorption with a B value of about 2.3.

TABLE 1.3
Dose-Normalized Relative Bioavailability (DN-RBA), IN v. IM, and Bile Salt
Enhancement Factor (EF).

| | | Bile Salt | Dose-Normalized Relative | | | | | |
|---|---|---|---|---|---|---|---|---|
| Formulation | | (STC) | Bioavailability (DN-RBA), | | | Mean | Bile | |
| No. | Epi Conc. | Conc. | IN v. IM | | | RBA | Salt | B = |
| (From Table 1) | (mg/mL) | (mg/mL) | AUC₀₋₃₀ₘᵢₙ | AUC₀₋₁₈₀ₘᵢₙ | Cₘₐₓ | v. IM | EF | EF*d |
| 1 | 2 | 4 | 6.7% | 6.6% | 5.0% | 6.1% | 1.6 | 0.1 |
| 2 | 2 | 6 | 22% | 16% | 21% | 20% | 5.2 | 0.3 |
| 3 | 2 | 8 | 58% | 45% | 59% | 54% | 14 | 0.7 |
| 4 | 2 | 10 | 67% | 47% | 77% | 63% | 17 | 0.8 |
| 5 | 4 | 0 | 2.3% | 6.4% | 2.6% | 3.8% | 1 | 0.1 |
| 6 | 4 | 4 | 7.2% | 13.5% | 4.8% | 8.5% | 2.3 | 0.2 |
| 7 | 4 | 6 | 30% | 24% | 32% | 29% | 7.6 | 0.8 |
| 8 | 4 | 8 | 44% | 35% | 45% | 41% | 11 | 1.1 |
| 9 | 4 | 10 | 40% | 28% | 50% | 39% | 10 | 0.5 |
| 10 | 8 | 4 | 15% | 19% | 11% | 15% | 4.0 | 0.8 |
| 11 | 8 | 6 | 28% | 26% | 24% | 26% | 6.9 | 1.4 |
| 12 | 8 | 8 | 88% | 61% | 106% | 85% | 23 | 2.3 |
| 13 | 10 | 6 | 48% | 33% | 63.3% | 48% | 12.8 | 1.3 |

FIG. 1 presents the Bile Salt EF results provided in Table 1.3. In particular, FIG. 1 is a graph of the Bile Salt (STC) Enhancement Factor as the Y-axis, entitled “Enhancement Factor, EF” versus Bile Salt (STC) concentration (mg/mL) (S) as the X-axis, entitled “Concentration of STC, mg/mL.” The dotted line in FIG. 1 is the curve fitting based on the plots of the Bile Salt EF results in Table 1.3. In FIG. 1, the 3 data points marked with squares are the smaller IN volume (12.5 μL) due to higher concentrations of the bile salt and the API. Notably, as shown by y=1.37x+100 in FIG. 1, the Bile Salt EF had an approximate linear relationship with the Bile Salt (STC) concentration (mg/mL) in rats, in particular, EF≈1.37S+1, where EF_{0-30/0-180/Cmax} is the Bile Salt EF_{0-30/0-180/Cmax}, and S is the Bile Salt (STC) concentration. Also, FIG. 1 shows that Bile Salts (STC) can enhance the absorption of an API (epinephrine) even at higher API concentrations such as 8-10 mg/mL epinephrine, thus demonstrating that bile salts can be effective as an absorption enhancer for IN delivery of APIs.

### Example 2—General Toxicity Study of Bile Salts as Absorption Enhancer for IN Delivery

In Example 2, an animal study was conducted to determine the general toxicity of bile salts. In Example 2, STC (hydrate) is the exemplary bile salt used, and epinephrine is the exemplary API used. Other suitable bile salts may also be utilized. In this general toxicity study, a total of 220 rats were assessed based on Groups A1-A5 detailed below:
Group A1: negative control (saline), number of rats (n)=44;
Group A2: 1 mg/mL epinephrine, 0 mg/mL STC, n=44;
Group A3: 1 mg/mL epinephrine, 5 mg/mL STC, n=44;
Group A4: 1 mg/mL epinephrine, 10 mg/mL STC, n=44;
Group A5: 1 mg/mL epinephrine, 15 mg/mL STC, n=44.

In each of Groups A1-A5, there are 44 rats, for which 24 rats were assessed 1 day after the IN administration and 20 rats were assessed 14 days after the IN administration. The purpose of this study was to assess the general toxicity of a bile salt (STC) through IN delivery so that the concentration of the API (epinephrine) was fixed at a low concentration of 1 mg/mL. Each rat was treated with two (2) IN administrations of the respective formulations in Groups A1-A5. The time interval between the 2 IN treatments was 15 minutes. No rats died during this general toxicity study before they were sacrificed.

The excipients in Groups A2-A5 include about 4 mg/mL citric acid (monohydrate), about 8 mg/mL sodium citrate (dihydrate), about 5.5 mg/mL chlorobutanol (hemihydrate), about 2-3 mg/mL sodium chloride, about 0.02 mg/mL EDTA (dihydrate), about 0.3 mg/mL sodium metabisulfite, and water for injection (q.s.). In addition, hydrochloric acid and sodium hydroxide may be added, as needed, to adjust the pH of the formulations to about 3.8.

The analysis of clinical pathology was conducted by a qualified veterinary laboratory (Quality Veterinary Laboratory (QVL), Davis, California, USA). QVL's clinical investigator concluded that there were no changes in hematology or clinical chemistry parameters observed on either Days 1 or 14, as compared to both saline and vehicle control groups (i.e. Groups A1-A2). In conclusion, two single IN administrations of the IN formulations in Groups A3-A5 between 15 minutes to rats did not result in changes in hematology or clinical chemistry parameters.

Histopathological evaluation was conducted by a qualified pathology laboratory (Experimental Pathology Laboratories, Inc. (EPL), Sterling, Virginia, USA). A total of 1,540 tissue samples from 220 rats, including (i) adrenal glands, (ii) brain, (iii) heart, (iv) kidneys, (v) liver, and (vi) lung lobes (left and right), were studied by EPL. EPL's histopathological findings were graded from 1 to 5, with 1 as minimal, and 5 as severe, depending upon severity. EPL's findings included an increased incidence of hemoglobin crystal/hemorrhage and an increased incidence of a mixed cell inflammation in the left and/or right lung lobes for the animals that were sacrificed on Day 1. The number of these changes observed was determined to be lower for the animals that were sacrificed on Day 14, showing recovery from the IN administration of the drugs.

The EPL investigator concluded that the maximum tolerated dose was considered to be at the Group A5 level (1 mg/mL epinephrine, 15 mg/mL STC). Due to the minimal severity of the pulmonary findings, and that these minor findings did not result in clinical signs of toxicity, the No-Observed-Adverse-Effect Level (NOAEL) was also considered to be at the Group A5 level.

The STC concentration in Group A5 is 15 mg/mL. Group A5 has 44 rats with an average body weight 0.298 kg on the treatment day. Based on studies conducted by EPL, QVL, and Amphastar, the NOAEL for the bile salt (STC) can be assessed as exceeding 15 mg/mL because 15 mg/mL was the highest studied concentration for IN administration. In this general safety study for STC, the highest dose of STC by an IN delivery is 0.75 mg (=15 mg/mL×0.025 mL×2), namely a relative dose of approximately 2.5 mg/kg (=0.75 mg/0.298 kg). Therefore, the NOAEL for bile salt (STC) at an IN delivery is 2.5 mg/kg or larger.

### Example 3-Local Irritation Study of Bile Salts as Absorption Enhancer for IN Delivery

In Example 3, an animal study was conducted to determine the local irritation of bile salts as an absorption enhancer for IN delivery, and more particularly, local irritation at the nasal mucosa. Example 3 investigates the histopathological effects of bile salt (STC) on nasal mucosa and damage reversibility in a rat model (n=378). Historically, bile salts have limited clinical use because of the irreversible damage to the mucosa and ciliotoxicity. Example 3 will demonstrate that the damage for nasal mucosa is reversible in 3-7 days at high dose for IN delivery. In Example 3, STC (hydrate) is the exemplary bile salt used, and epinephrine is the exemplary API used. Other suitable bile salts may also be utilized.

In this study, a total of 378 rats were assessed based on Groups B1-B5 detailed in Table 3.1. Note that the “No.” columns in Tables 3.1-3.3 are the same.

TABLE 3.1
IN Formulations Tested for Nasal Mucosa Irritation Study.

| | | | Total number | | | Bile Salt |
|---|---|---|---|---|---|---|
| | | | of sprays (K) | | Epi | Conc. |
| | | Arm | (25 μL in | B = | Conc. | (STC) |
| No. | Group | Code | each spray) | d * EF * K | (mg/mL) | (mg/mL) |
| 1 | B1 | B | 1 | N/A | 0 | 0 |
| 2 | B2 | T0 | 1 | 0.025 | 1 | 0 |
| 3 | B3 | T1 | 1 | 0.20 | 1 | 5 |
| 4 | B4 | T2a | 1 | 0.37 | 1 | 10 |
| 5 | | | | | | |
| 6 | | | | | | |
| 7 | | | | | | |
| 8 | B5 | T2b | 2 (2 sprays | 0.74 | 1 | 10 |
| 9 | | | in 1 day) | | | |
| 10 | | | | | | |
| 11 | | | | | | |
| 12 | B6 | T2c | 6 (2 sprays | 2.2 | 1 | 10 |
| 13 | | | each day for | | | |
| 14 | | | 3 consecutive | | | |
| 15 | | | days) | | | |
| 16 | B7 | T3 | 6 (2 sprays | 3.2 | 1 | 15 |
| 17 | | | each day for | | | |
| 18 | | | 3 consecutive | | | |
| 19 | | | days) | | | |

TABLE 3.2
Dose of Formulations Tested for Nasal Mucosa Irritation Study.

| | | Bile Salt | | | | | | |
|---|---|---|---|---|---|---|---|---|
| No. | | Conc. | | | | | Dose of Bile | Number |
| (From | Epi Conc. | (STC) | Mean Body | Dose of Epi | | | Salt (STC) | of rats |
| Table 4) | (mg/mL) | (mg/mL) | Weight (kg) | d, mg | mg/kg | MRDH | (kg/mg) | (n) |
| 1 | 0 | 0 | 0.24 | 0 | 0 | 0 | 0 | 20 |
| 2 | 1 | 0 | 0.26 | 0.025 | 0.10 | 10 | 0.0 | 20 |
| 3 | 1 | 5 | 0.27 | 0.025 | 0.09 | 9 | 0.5 | 20 |
| 4 | 1 | 10 | 0.31 | 0.025 | 0.08 | 8 | 0.8 | 20 |
| 5 | | | 0.26 | 0.025 | 0.09 | 9 | 0.9 | 20 |
| 6 | | | 0.25 | 0.025 | 0.10 | 10 | 1.0 | 20 |
| 7 | | | 0.22 | 0.025 | 0.11 | 11 | 1.1 | 20 |
| 8 | 1 | 10 | 0.27 | 0.05 | 0.19 | 19 | 1.9 | 20 |
| 9 | | | 0.27 | 0.05 | 0.19 | 19 | 1.9 | 20 |
| 10 | | | 0.25 | 0.05 | 0.20 | 20 | 2.0 | 20 |
| 11 | | | 0.23 | 0.05 | 0.22 | 22 | 2.2 | 20 |
| 12 | 1 | 10 | 0.30 | 0.15 | 0.50 | 50 | 5.0 | 20 |
| 13 | | | 0.27 | 0.15 | 0.55 | 55 | 5.5 | 20 |
| 14 | | | 0.25 | 0.15 | 0.59 | 59 | 5.9 | 20 |
| 15 | | | 0.23 | 0.15 | 0.65 | 65 | 6.5 | 19 |
| 16 | 1 | 15 | 0.30 | 0.15 | 0.50 | 50 | 7.5 | 20 |
| 17 | | | 0.28 | 0.15 | 0.55 | 55 | 8.2 | 20 |
| 18 | | | 0.26 | 0.15 | 0.58 | 58 | 8.7 | 20 |
| 19 | | | 0.23 | 0.15 | 0.65 | 65 | 9.8 | 19 |

Because this study is for assessment of nasal mucosa irritation by bile salt (STC), the concentration of the API (epinephrine) was fixed at 1 mg/mL. The IN delivery volume for each spray is 25 μL. The rats were treated by one (1) IN spray (Arms T1, T2a), two (2) IN sprays (Arm T2b), and six (6) IN sprays (Arms T2c and T3). The six-spray treatments in Arms T2c and T3 were conducted as two sprays a day for 3 days consecutively. These two types of treatments, Arms T2c and T3, with six (6) IN sprays (as shown in Table 4) were designed for the extreme test of the nasal irritation caused by bile salt (STC).

Also in Table 4, the quantity B represents the net API epinephrine absorption, which is defined as B=d×EF×K, where d is the dose of epinephrine (in mg), EF is the Bile Salt EF, and K is the number of sprays of the IN formulation. Also in Table 5, MRDH is the Maximum Relative Dose for Human for Epi-pen®, 0.01 mg/kg.

Also, for Example 3, the excipients (not shown in Table 4) in these formulations include about 4 mg/mL citric acid (monohydrate), about 8 mg/mL sodium citrate (dihydrate), about 5.5 mg/mL chlorobutanol (hemihydrate), about 2-3 mg/mL sodium chloride, about 0.02 mg/mL EDTA (dihydrate), about 0.3 mg/mL sodium metabisulfite, and water for injection (q.s.). In addition, hydrochloric acid and sodium hydroxide may be added, as needed, to adjust the pH of the formulations to about 3.8.

The histopathology of the nasal tissue was examined to evaluate nasal mucosa tolerance to each test articles with respect to bile salt (STC) amounts. In order to study the damage recoverability, the rat histopathological studies were conducted at 4 hours, 3 days, 1 week, and 2 weeks after the last treatments for Arms-T2a, T2b, T2c, and T3, as summarized in Table 3.3.

TABLE 3.3
Results of Nasal Mucosa Irritation Study Using Bile Salt As Absorption
Enhancer for IN Delivery.

| No. | | Bile Salt | | | | | | Average | | |
|---|---|---|---|---|---|---|---|---|---|---|
| (From | Epi | Conc. | | | | | | Irritation Per | | |
| Table | Conc. | (STC) | Total Number of Findings | | | | | Rat** | | |
| 4) | (mg/mL) | (mg/mL) | m_{g=1} | m_{g=2} | m_{g=3} | m_{g=4} | TIP* | M^{1,2} | M^{3,4} | P |
| 1 | 0 | 0 | 12 | 5 | 0 | 0 | 22 | 0.9 | 0.0 | 1.1 |
| 2 | 1 | 0 | 6 | 0 | 0 | 0 | 6 | 0.3 | 0.0 | 0.3 |
| 3 | 1 | 5 | 13 | 0 | 0 | 0 | 13 | 0.7 | 0.0 | 0.7 |
| 4 | 1 | 10 | 14 | 11 | 9 | 2 | 71 | 1.3 | 0.6 | 3.6 |
| 5 | | | 18 | 1 | 0 | 0 | 20 | 1.0 | 0.0 | 1.0 |
| 6 | | | 3 | 0 | 0 | 0 | 3 | 0.2 | 0.0 | 0.2 |
| 7 | | | 7 | 0 | 0 | 0 | 7 | 0.4 | 0.0 | 0.4 |
| 8 | 1 | 10 | 37 | 16 | 8 | 2 | 101 | 2.7 | 0.5 | 5.1 |
| 9 | | | 32 | 5 | 0 | 0 | 42 | 1.9 | 0.0 | 2.1 |
| 10 | | | 8 | 1 | 0 | 0 | 10 | 0.5 | 0.0 | 0.5 |
| 11 | | | 10 | 0 | 0 | 0 | 10 | 0.5 | 0.0 | 0.5 |
| 12 | 1 | 10 | 31 | 18 | 4 | 0 | 79 | 2.5 | 0.2 | 4.0 |
| 13 | | | 40 | 21 | 5 | 0 | 97 | 3.1 | 0.3 | 4.9 |
| 14 | | | 14 | 0 | 0 | 0 | 14 | 0.7 | 0.0 | 0.7 |
| 15 | | | 6 | 1 | 0 | 0 | 8 | 0.4 | 0.0 | 0.4 |
| 16 | 1 | 15 | 69 | 22 | 14 | 11 | 199 | 4.6 | 1.3 | 10.0 |
| 17 | | | 38 | 46 | 12 | 0 | 166 | 4.2 | 0.6 | 8.3 |
| 18 | | | 33 | 3 | 0 | 0 | 39 | 1.8 | 0.0 | 2.0 |
| 19 | | | 9 | 1 | 0 | 0 | 11 | 0.5 | 0.0 | 0.6 |

In Table 3.3, TTP is Total irritation points, defined as p=m₁+2 m₂+3 m₃+4 m₄, mj is the number of observations with Grade-j; M^{1,2}=the average of m₁+m₂; M_{3,4}=average of m₃+m₄; P-average of TTP.

The histopathological evaluation for Example 3 was conducted by a qualified pathology laboratory (Experimental Pathology Laboratories, Inc. (EPL) in Sterling, Virginia, USA). In total, EPL assessed 55 types of microscopic findings in Turbinate I to IV. The severity of the microscopic findings was reported as Grade 1 to 5 by EPL as follows: Grade 1 Minimal.

Grade 2 Mild—a noticeable but not a prominent feature of the tissue.

Grade 3 Moderate—a prominent but not a dominant feature of the tissue.

Grade 4 Marked—a dominant but not an overwhelming feature of the tissue.

Grade 5 Severe—an overwhelming feature of the tissue.

No Grade 5 finding was reported for this nasal irritation study among all 378 samples examined for 20,790 (=55×378) evaluated histopathological items. The total number of findings with Grades 1, 2, 3, 4 (denoted as mg=1, mg=2, mg=3, and mg=4, respectively, note no g=5 finding) for each arm at each assessment time are summarized in Table 3.3.

EPL concluded that a dose-related increase in the nasal cavity epithelial, inflammatory, and exudative changes was typically seen unilaterally after exposure to STC and Epi and was most evident at 4 hours in groups receiving ≥10 mg/mL STC with more than one administration, although Group B4 receiving 10 mg/mL STC was also affected fairly uniformly at a lesser severity than Groups B5-7 at 4 hours. Rapid repair from widespread erosion/flattening of respiratory epithelium was evident as respiratory epithelial cell hyperplasia with concomitant decreased cilia and less exudate and inflammation at three days. The repair progressed to sporadic findings at 1 and 2 weeks with many of the distal nasal cavities (Levels III and IV) from Groups B4-B7 being normal at 2 weeks post-dose.

### Quantitative Analysis of Data for Local Tolerance Histopathological Findings in Example 3

In order to quantitatively analyze the data for histopathological findings the two (2) quantities, (i) total irritation point (TIP), denoted asp, and (ii) number of findings with Grades 3 and 4, denoted as m^{3,4}, among different treatment groups were analyzed. For a given rat belonging to a given arm and evaluation time, TIP and m^{3,4} are defined as follows:

p m₁+2m₂+3m₃+4m₄ (Equation 1)

and

m^{3,4}m₃+m₄ (Equation 2)

where m₁, m₂, m₃, m₄ are number of findings with Grade 1, 2, 3, 4, respectively, from the 55 microscopic histopathological items assessed by EPL. In the definition of TIP, higher grade has a greater weight, for example the weight for m₁ (number of Grade-1 findings) is 1 and the weight for m⁴ (number of Grade-4 findings) is 4 in the definition of TIP as demonstrated in Equation 1.

TIP represents the global findings of local irritation caused by the bile salt (STC), and m^{3,4} reflects number of finding with higher grades. The average of TIP and m^{3,4}, are denoted as P and M^{3,4}, respectively, for a given arm and evaluation time:

$$P = \frac{1}{n} {\sum }_{j = 1}^{n} {p}_{j} \qquad ( Equation 3 )$$

$$and$$

$${M}^{3 , 4} = \frac{1}{n} {\sum }_{j = 1}^{n} {m}_{j}^{3 , 4} \qquad ( Equation 4 )$$

where n is the number of rats for the given treatment arm and given assessment time (4 hrs, 3 days, 1 week or 2 weeks). The quantitative data of P and M^{3,4} are provided in Table 3.3.

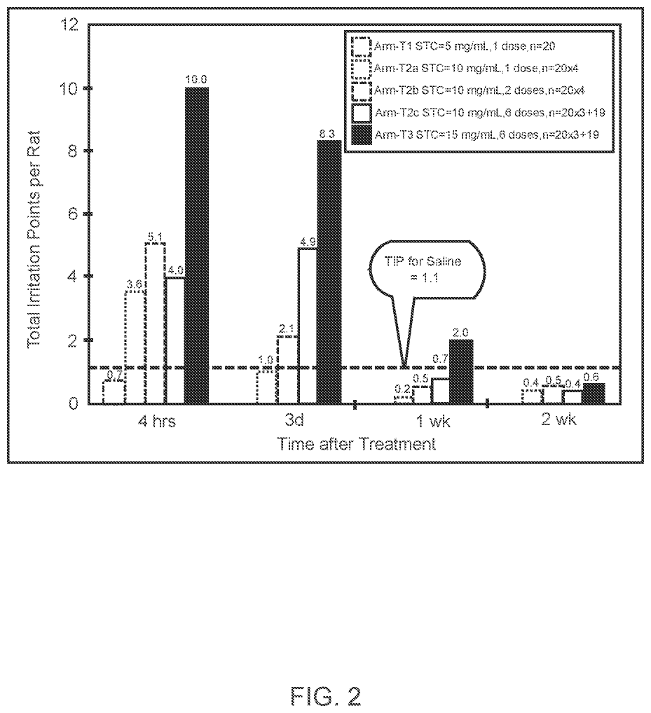

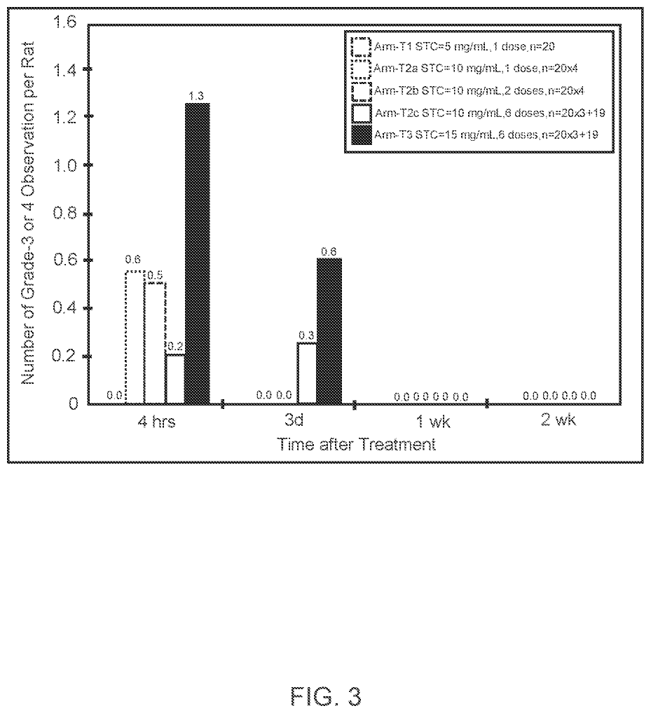

FIGS. 2-3 demonstrate the average TIP and M^{3,4}, respectively, for Arms-T1, T2a, T2b, T2c, and T3, at evaluation times of 4 hrs, 3 days, 1 week, and 2 weeks, respectively. Notably, the following profile is observed from FIGS. 2-3, which are consistent with the conclusions drawn by EPL.
More irritation was observed for Arm-T3, 15 mg/mL;
For 1 or 2 IN sprays, 3 days after treatment, a quick reduction of the findings shows a “rapid repair”;
For 6 IN sprays (an extreme test) with STC=10 mg/mL, 1 week after treatment, the TIP reduced to even below the saline treatment;
For 6 IN sprays (an extreme test) with STC=15 mg/mL, 2 week after treatment, the TIP reduced to even below that of the saline treatment; and
One (1) week after treatment, the M for all treatments became zero.

It is also notable that the highest STC dose treated in this local irritation study is 9.8 mg/kg, which is approximately 3.9 times of the NOAEL of 2.5 mg/kg that was observed in the general toxicity study for STC using an IN delivery.

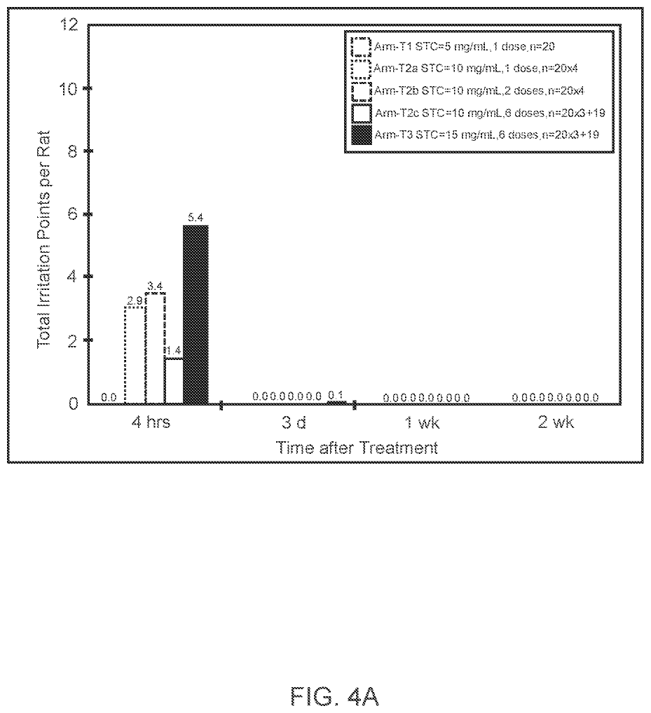

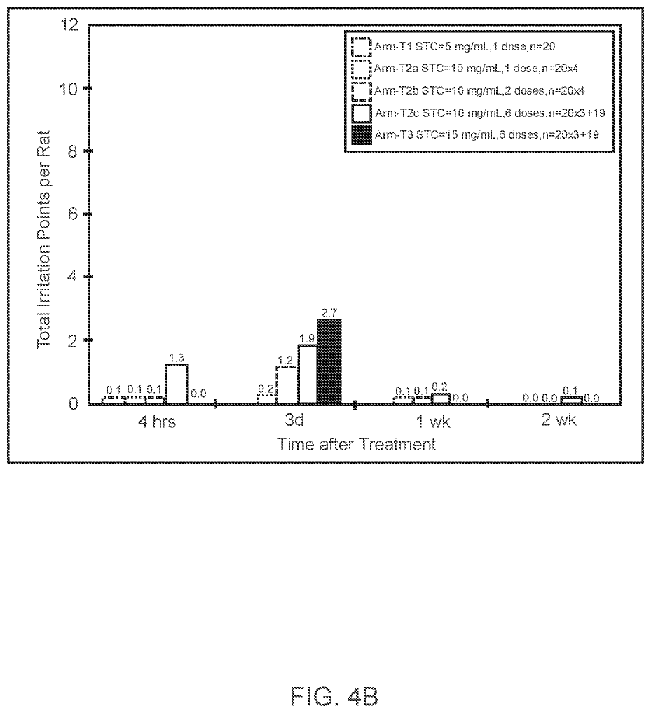

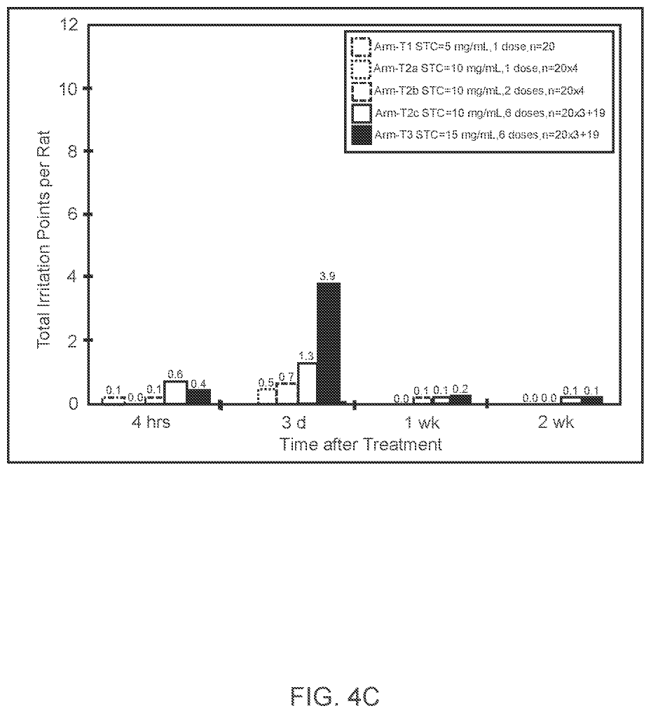

Among the 55 histopathological items assessed by EPL, the most frequent findings are (i) erosion/flattening, (ii) decreased cilia, and (iii) hyperplasia of respiratory epithelium. These 3 findings (the sum of observation in both turbinate I and II) are 63% f all findings based on the TIP assessment. FIG. 4A-4B show the TIP for these three (3) types of findings.

FIG. 4A indicates that the TIP of erosion/flattening of respiratory epithelium reached the peak of the finding immediately (4 hrs after treatment), and can be rapidly repaired in 3 days, even for six (6) repeated sprays of the IN formulations at high STC concentration of 10 or 15 mg/mL (Arms T2c and T3).

FIG. 4B-4C indicate that the TIP of the decreased cilia and hyperplasia, respectively of respiratory epithelium, both reached the peak of the findings on Day 3 after treatment, and can be repaired in 1 week, even for six (6) repeated sprays of the IN formulations at high STC concentration of 10 or 15 mg/mL (Arms T2c and T3). Thus, all damages/findings can be repaired or reversed in one (1) week to the negative control group level, as concluded by EPL. Therefore, Example 3 demonstrates that the damages of “decreased cilia” are reversible based on this experimental study as demonstrated in FIG. 4B.

### Example 4-Bile Salts as Absorption Enhancer for IN Delivery of Epinephrine

Example 4 is an animal study using epinephrine as the API, and sodium taurochenodeoxycholate (STCDC) (0-10 mg/mL) and sodium taurocholate (STC) (0-20 mg/mL) as the bile salts for enhancing the absorption of epinephrine. Also studied is the IM control of 1 mg/mL epinephrine. This study was designed to study the absorption enhancement effect of taurochenodeoxycholate (TCDC) and taurocholate (TC). Table 4.1 details the formulations tested for Example 4, and Tables 4.1 and 4.2 provide the PK results.

TABLE 4.1
Formulations Tested for Example 4 (Excipients Not Shown)

| | | Epi Conc. | | | | |
|---|---|---|---|---|---|---|
| Formu- | | (mg/mL)/ | Bile Salt Conc. | | Dose | Number |
| lation | Delivery | Dose | (mg/mL)/Dose | | Volume | of rats |
| No. | Route | (mg) | (mg) | pH | (μL) | (n=) |
| 1 | IM | 1/0.025 | 0/0 | 3.6 | 25 | 6 |
| 2 | IN | 1/0.025 | 0/0 | 3.6 | 25 | 6 |
| 3 | IN | 1/0.025 | STC: 2/0.05 | 3.6 | 25 | 6 |
| 4 | IN | 1/0.025 | STC: 5/0.125 | 3.6 | 25 | 6 |
| 5 | IN | 1/0.025 | STC: 10/0.25 | 3.6 | 25 | 6 |
| 6 | IN | 0.5/ | STC: 20/0.05 | 3.6 | 25 | 6 |
| | | 0.0125 | | | | |
| 7 | IN | 1/0.025 | 0/0 | 3.6 | 25 | 6 |
| 8 | IN | 1/0.025 | STCDC: 2/0.05 | 3.6 | 25 | 6 |
| 9 | IN | 1/0.025 | STCDC: 5/0.125 | 3.6 | 25 | 6 |
| 10 | IN | 1/0.025 | STCDC: 10/0.25 | 3.6 | 25 | 6 |

In Table 4.1, Formulations Nos. 1-10 had excipients of about 8.5 mg/mL sodium chloride, about 3.84 mg/mL citric acid, about 1.5 mg/mL sodium metabisulfite, and pH adjustors (HCl 10% w/v, NaOH) as needed to adjust the pH to 3.6.

For the IN delivery, test article was intranasally (IN) delivered in the amount of 25 μL to the right nostril of the rat using a 27G Blunt Needle (Instech, Part No. LS27 or equivalent) connected to a glass syringe (Hamilton, Model 1725, 250 μL or equivalent). The rat was anesthetized to effect (isoflurane, 5% for approximately 5 minutes) before administration and returned to its cage after the administration.

For the IM administration, test article was intramuscularly injected in the amount of 25 μL to the right back thigh of the rat using a 31G insulin syringe (BD Insulin Syringe, 0.3 mL, ¹² unit). To be consistent with the IN administration, the rats that received IM injection was also anesthetized to effect (isoflurane, 5% for approximately 5 minutes) before injection and returned to their cage after the injection.

Subsequently, the plasma samples from the rats were drawn post-dose (IM and IN) at 0 minute, 5 minutes, 10 minutes, 15 minutes, 30 minutes, 60 minutes, 120 minutes, and 180 minutes. The drawn plasma samples were analyzed for the PK results, as shown below.

TABLE 4.2
Summary of PK Results for Example 4.

| Formulation | | | | | | | |
|---|---|---|---|---|---|---|---|
| No. (From | Cₘₐₓ | tₘₐₓ, | AUC, ng/mL * min | | | | Epi Dose |
| Table 7) | (ng/mL) | min | AUC_{0-10min.} | AUC_{0-30min.} | AUC_{0-60min.} | AUC_{0-30min.}/mg | (mg/kg) |
| 1 | 7.6 | 9 | 39.9 | 133 | 235 | 5332 | 0.092 |
| 2 | 1.1 | 75 | 2 | 12 | 32 | 484 | 0.094 |
| 3 | 0.7 | 65 | 3 | 12 | 31 | 493 | 0.093 |
| 4 | 1.7 | 18 | 9 | 27 | 51 | 1085 | 0.097 |
| 5 | 7.7 | 5 | 48 | 91 | 127 | 3659 | 0.103 |
| 6 | 3.8 | 9 | 24 | 65 | 111 | 5180 | 0.052 |
| 7 | 0.1 | 23 | 0.1 | 1 | 3 | 47 | 0.077 |
| 8 | 1.9 | 10 | 12 | 35 | 61 | 1386 | 0.076 |
| 9 | 8.9 | 8 | 58 | 117 | 164 | 4672 | 0.077 |
| 10 | 7.4 | 9 | 48 | 124 | 190 | 4957 | 0.082 |

TABLE 4.3
Relative Bioavailability (RBA), IN vs. IM for Example 4.

| Formulation | | | | |
|---|---|---|---|---|
| No. (From | Relative Bioavailability (RBA), IN vs. IM | | | Mean RBA, |
| Table 7) | AUC_{0-30 min.} | AUC_{0-60 min.} | Cₘₐₓ | IN v. IM |
| 2 | 9% | 14% | 14% | 12% |
| 3 | 9% | 13% | 10% | 11% |
| 4 | 20% | 22% | 22% | 21% |
| 5 | 69% | 54% | 101% | 75% |
| 6 | 97% | 95% | 100% | 97% |
| 7 | 1% | 1% | 1% | 1% |
| 8 | 26% | 26% | 25% | 26% |
| 9 | 88% | 70% | 116% | 91% |
| 10 | 93% | 81% | 97% | 90% |

In Table 4.1, the Mean RBA is the average of the RBAs for AUC₀₋₃₀ₘᵢₙ, AUC₀₋₆₀ₘᵢₙ, and Cₘₐₓ. Notably, as demonstrated in Table 4.1, Formulation No. 6 provides the highest mean RBA at 97% with respect to Formulation No. 1 (IM), thereby demonstrating that IN delivery can deliver a similar amount of epinephrine as in the IM route. In addition, as shown in Table 4.1, Formulation No. 6 has a similar tₘₐₓ as Formulation No. 1 (IM).

Also notable, as shown in Table 4.1, Formulation Nos. 9 and 10 also have a similar mean RBA, at 910% and 900% respectively, compared to Formulation No. 1 (IM), thereby demonstrating that IN delivery using STCDC as a bile salt can deliver a similar amount of epinephrine as in the IM route. In addition, as shown in Table 4.1, Formulation Nos. 9 and 10 has a similar tₘₐₓ as Formulation No. 1 (IM).

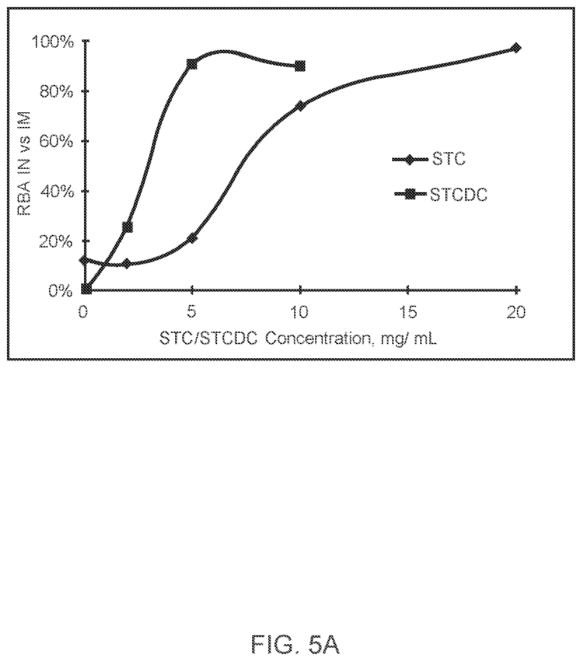

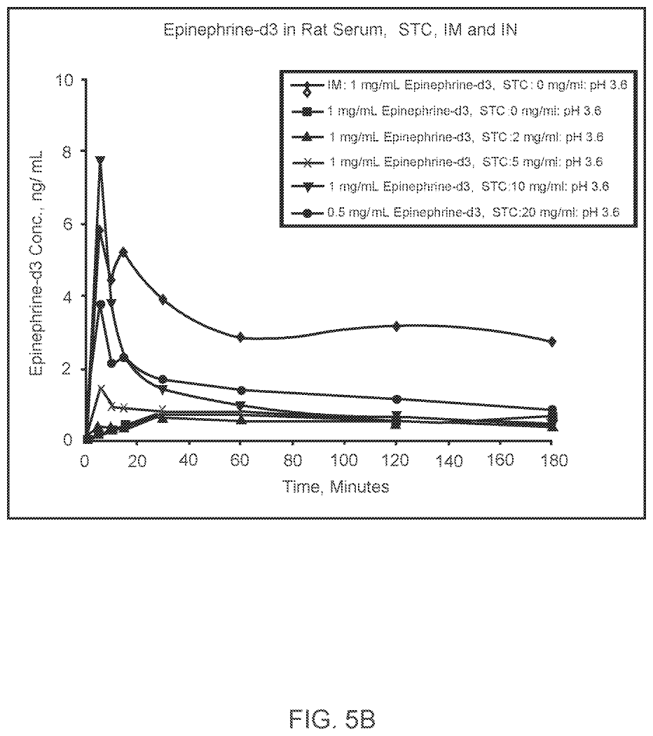

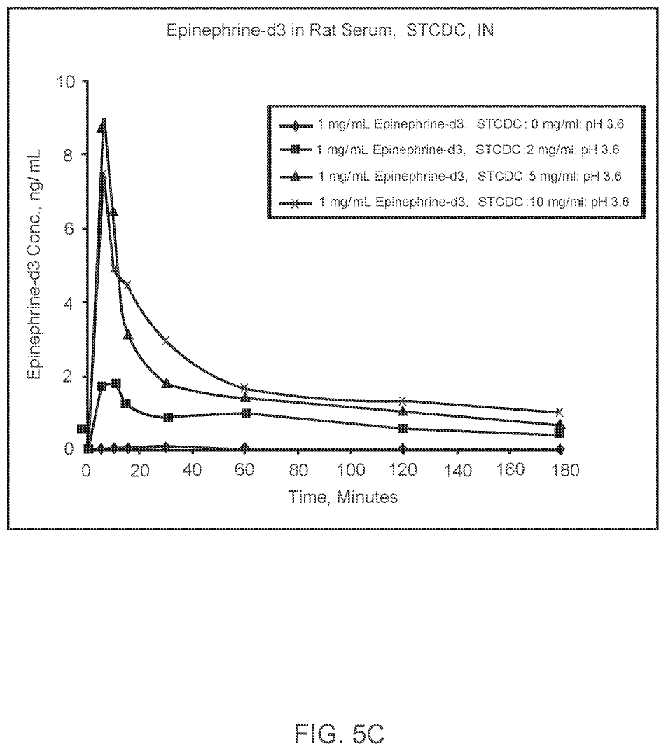

Additionally, FIG. 5A-5C depict some of the key PK results shown in Tables 4.2 and 4.3. FIG. 5A is a graph illustrating the relative bioavailability of epinephrine, IN versus IM, in which the IN delivery uses either STC or STCDC. Notably, as shown in FIG. 5A, STCDC has better absorption enhancement effect than STC. For example, at a concentration of 5 mg/mL, the RBA was 91% for STCDC compared to STC's 21%.

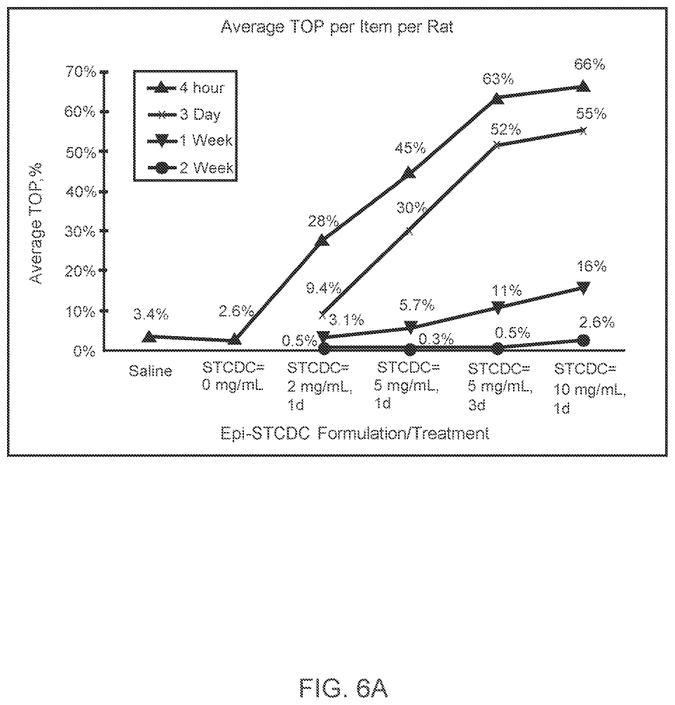

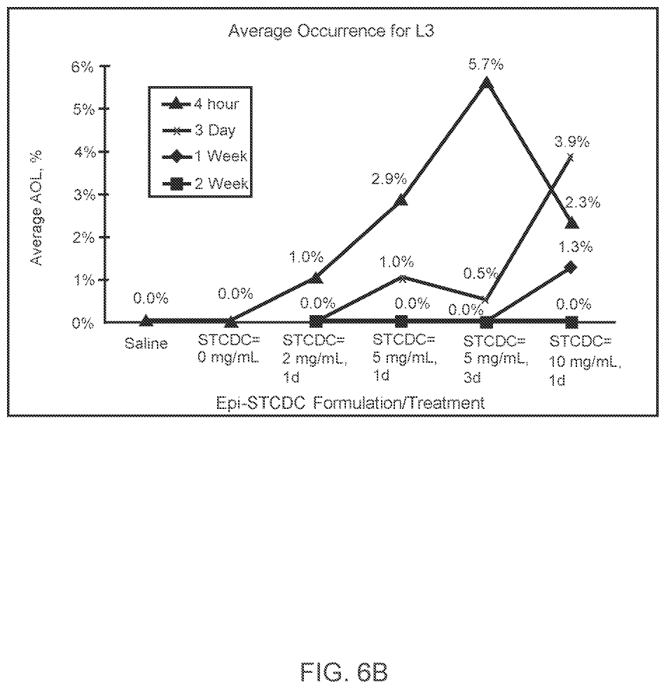

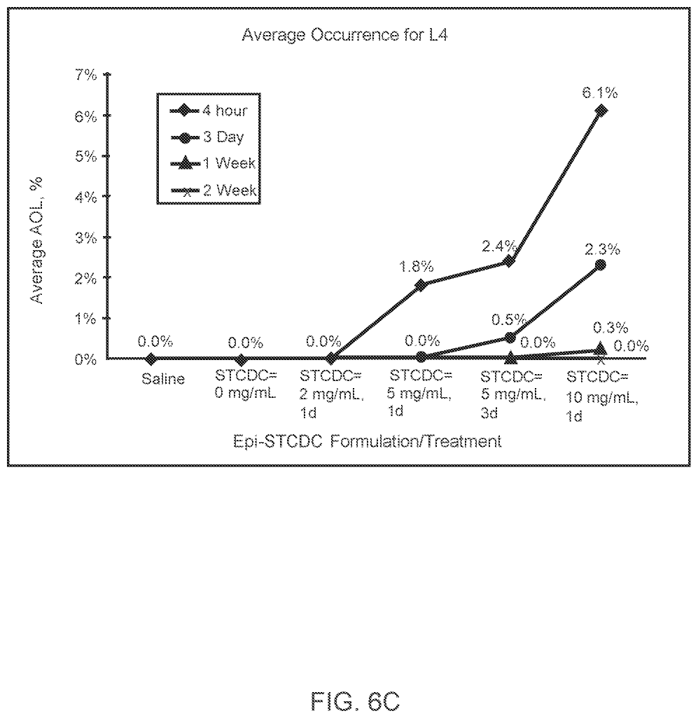

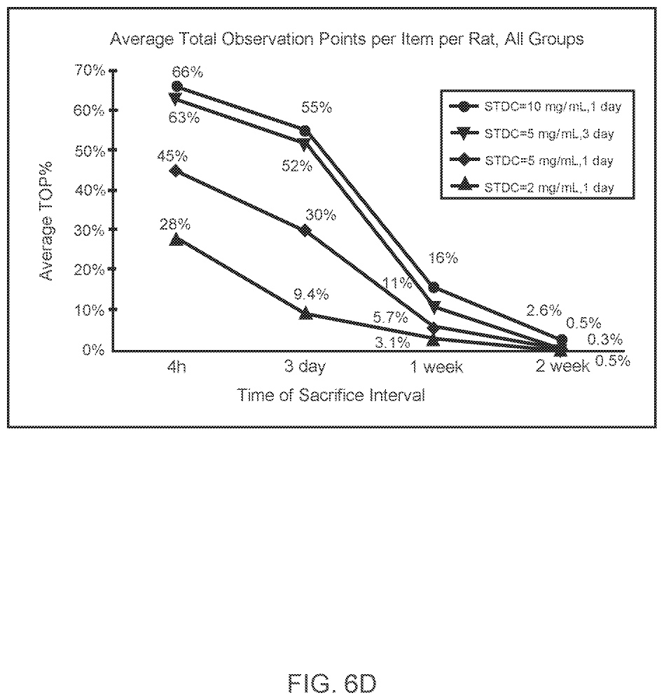

FIG. 5B is a graph illustrating the mean epinephrine concentration in rat serum from 0 min. to 180 mins. utilizing STC as the bile salt. FIG. 5C is a graph illustrating the mean epinephrine concentration in rat serum from 0 min. to 180 mins. utilizing STCDC as the bile salt. Notably, as shown in FIG. 6B-6C, when there is no bile salt in the formulation (see Formulation Nos. 2 and 7 of Table 4.1), the epinephrine bioavailability relative to IM administration (Formulation No. 1 of Table 4.1) was only 1-12%. Therefore, it can be concluded that adding a bile salt, such as STC or STCDC, to the formulation can enhance the epinephrine absorption into the bloodstream during IN delivery.

### Example 5-Local Toxicity Study of Bile Salt (STCDC) as Absorption Enhancer for IN Delivery of Epinephrine

Example 5 is an animal study designed to investigate the possible histological effects of bile salts (STCDC) on nasal mucosa of rats when the bile salt is used as an absorption enhancer for IN delivery. Table 5.1 shows the various IN formulations tested in Example 5, with STCDC as the representative bile salt absorption enhancer and epinephrine as the representative API. These IN formulations were administered intranasally to the rats. The histopathology of the rat's nasal tissue was examined to evaluate nasal mucosa tolerance to these tested IN formulations.

TABLE 5.1
Formulations Tested for Example 5 (Excipients Not Shown).

| | Epi Conc. (mg/mL)/ | STCDC Conc. (mg/mL)/Dose |
|---|---|---|
| Group No. | Dose (mg) | (mg) |
| 1 (Negative Control) | 0/0 | 0/0 |
| 2 | 1/0.025 | 0/0 |
| 3 | 1/0.025 | 2/0.05 |
| 4 | 1/0.025 | 5/0.125 |
| 5 | 1/0.025 | 5/0.125 |
| 6 | 1/0.025 | 10/0.25 |

In Table 5.1, Group Nos. 2-6 each had excipients of about 8.5 mg/mL sodium chloride, about 3.84 mg/mL citric acid, about 1.5 mg/mL sodium metabisulfite, about 2.3 mg/mL HCl (10% w/v), and a pH adjustor (NaOH) as needed to adjust the pH to 3.6. Group No. 1 is the negative control and is a saline nasal spray (CVS Health, Lot 6EK0606, Exp. 04/18) containing purified water, 0.65% of sodium chloride, disodium phosphate, phenylcarbinol, monosodium phosphate, and benzalkonium chloride as preservatives.

TABLE 5.2
IN Dose Treatment for Example 5.

| Group | | | | | | | |
|---|---|---|---|---|---|---|---|
| No. | | | | Sampling After Last | | | |
| (from | Number | | | Treatment (# of rats) | | | |
| Table | of rats | IN Dose | IN | 4 | 3 | 1 | 2 |
| 5.1) | (n=) | Frequency | Treatment | hrs | days | week | weeks |
| 1 | 8 | 2 × 25 μL | IN for | 8 | N/A | N/A | N/A |
| (Neg. | | | 1 nostril | | | | |
| Cont.) | | | (25 μL), after | | | | |
| 2 | 8 | 2 × 25 μL | 15 seconds, | 8 | N/A | N/A | N/A |
| 3 | 32 | 2 × 25 μL | one more | 8 | 8 | 8 | 8 |
| 4 | 32 | 2 × 25 μL | IN to same | 8 | 8 | 8 | 8 |
| | | | nostril | | | | |
| | | | (25 μL) | | | | |
| 5 | 32 | 2 × 25 μL × 3 | ibid, | 8 | 8 | 8 | 8 |
| | | | but for 3 | | | | |
| | | | consecutive | | | | |
| | | | days | | | | |
| 6 | 32 | 2 × 25 μL | ibid, for | 8 | 8 | 8 | 8 |
| | | | 1 day | | | | |

One hundred and forty four (144) rats (Male:Female=1:1) are randomly divided into six groups as listed in Table 5.2. Groups 1-2 each have four male and four female rats, while Groups 3-6 each had sixteen male and sixteen female rats. Each formulation is IN delivered in the amount of 25 μL to the right nostril using a 27G Blunt Needle (Instech, Part No. LS27 or equivalent) connected to a glass syringe (Hamilton, Model 1725, 250 μL or equivalent). The rat is anesthetized (isoflurane) before administration; remains under anesthesia for 3 minutes after administration and then returns to its cage. Fifteen (15) minutes after the first administration, the same test article is again intranasally delivered in the same amount of 25 μL to the same right nostril using the same procedures.

For rats in Group No. 5 of Table 5.2, the IN administrations are continued for the total of three consecutive days so that each rat receives six intranasal administrations of the Group No. 4. Rats in all other groups received only two intranasal administrations of the respective article.

Four (4) male and four (4) female rats were sacrificed by carbon dioxide at four time points (4 hr, 3 days, 1 week, and 2 weeks) after the last treatment, as specified in Table 5.2. The nasal passage/nasopharynx tissues was taken out. To help preserve the turbinate epithelium, formalin via injected into the nasopharyngeal opening until it came out of nares, then the whole tissue was immersed in 10% neutral buffered formalin. The tissue samples were sent to a qualified pathology laboratory (Experimental Pathology Laboratories, Inc. (EPL)) for histopathologic evaluation.

In total, EPL assessed 48 types of microscopic findings in nasal turbinate cavity level I through IV, as detailed in Table 5.3.

TABLE 5.3
Summary of 48 Histopathologic Observations

| Nasal | |
|---|---|
| Cavity | Item of Histopathologic Observation |
| Nasal | 1. Decreased; Cilia; Respiratory Epithelium |
| Cavity I | 2. Erosion/Flattening; Olfactory Epithelium |
| | 3. Erosion/Flattening; Respiratory Epithelium |
| | 4. Exudate; Serocellular; Meatus |
| | 5. Hemorrhage; Meatus |
| | 6. Hyperplasia; Respiratory Epithelium |
| | 7. Increased Mucous Cells; Respiratory Epithelium |
| | 8. Infiltrate; Neutrophils; Septum/Turbinates |
| | 9. Necrosis; Individual Cell; Olfactory |
| Nasal | 1. Decreased; Cilia; Respiratory Epithelium |
| Cavity II | 2. Degeneration; Olfactory Epithelium |
| | 3. Erosion/Flattening; Olfactory Epithelium |
| | 4. Erosion/Flattening; Respiratory Epithelium |
| | 5. Exudate; Serocellular; Meatus |
| | 6. Foreign Material; Meatus |
| | 7. Hemorrhage; Meatus |
| | 8. Hyperplasia; Respiratory Epithelium |
| | 9. Increased Mucous Cells; Respiratory Epithelium |
| | 10. Infiltrate; Neutrophils; Septum/Turbinates |
| | 11. Necrosis; Individual Cell; Olfactory Epithelium |
| Nasal | 1. Decreased; Cilla; Respiratory Epithelium; Nasopharynx |
| Cavity III | 2. Degeneration; Olfactory Epithelium |
| | 3. Diverticulum; Respiratory Epithelium; Septum |
| | 4. Erosion/Flattening; Epithelium; Nasopharynx |
| | 5. Erosion/Flattening; Olfactory Epithelium |
| | 6. Erosion/Flattening; Respiratory Epithelium |
| | 7. Exudate; Serocellular; Meatus |
| | 8. Exudate; Serocellular; Nasopharynx |
| | 9. Hemorrhage; Meatus |
| | 10. Hyperplasia; Epithelium; Nasopharynx |
| | 11. Hyperplasia; Respiratory Epithelium |
| | 12. Infiltrate; Mixed Cell; Lateral Wall/Turbinate |
| | 13. Infiltrate; Neutrophils; Nasopharynx |
| | 14. Infiltrate; Neutrophils; Septum/Turbinates |
| | 15. Necrosis; Individual Cell; Olfactory Epithelium |
| Nasal | 1. Decreased; Cilla; Respiratory Epithelium; Nasopharynx |
| Cavity IV | 2. Degeneration; Olfactory Epithelium |
| | 3. Erosion/Flattening; Epithelium; Nasopharynx |
| | 4. Erosion/Flattening; Olfactory Epithelium |
| | 5. Erosion/Flattening; Respiratory Epithelium |
| | 6. Exudate; Serocellular; Meatus |
| | 7. Exudate; Serocellular; Nasopharynx |
| | 8. Hemorrhage; Meatus |
| | 9. Hyperplasia; Epithelium; Nasopharynx |
| | 10. Hyperplasia; Respiratory Epithelium |
| | 11. Infiltrate; Neutrophils; Nasopharynx |
| | 12. Infiltrate; Neutrophils; Septum/Turbinates |
| | 13. Necrosis; Individual Cell; Olfactory Epithelium |

The severity of the microscopic findings was reported as Level 1 to 5 by EPL as follows:
Level 1 (L-1) Minimal—inconspicuous to barely noticeable but so minor, small, or infrequent.
Level 2 (L-2) Mild—a noticeable but not a prominent feature of the tissue.
Level 3 (L-3) Moderate—a prominent but not a dominant feature of the tissue.
Level 4 (L-4) Marked—a dominant but not an overwhelming feature of the tissue.
Level 5 (L-5) Severe—an overwhelming feature of the tissue.

In order to quantitatively analyze the data for histopathological findings, the three (3) quantities, (i) total observation point (TOP), denoted as TOP, (ii) Average TOP per item per rat, and (iii) average occurrence for Level i (AOL)-i (i=1-5) were analyzed.

TOP=m₁+2m₂+3m₃+4m₄+5m₅ (1)

AverageTOP=TOP/48/n (2)

AOL-i=mᵢ/48/n (3)

where m₁, m₂, m₃, m₄, and m₅ are number of findings with Level 1, 2, 3, 4, 5, respectively, from the 48 microscopic histopathological items (as detailed in Table 5.3) assessed by EPL, n is the number of rats examined. In the definition of TOP, higher grade has a greater weight, for example the weight for m1 (number of Level-1 findings) is 1 and the weight for m₄ (number of Grade-4 findings) is 4 in the definition of TOP as demonstrated in Eq. (1). Results of Average TOP and AOL for Level 3 (L-3), Level 4 (L-4), and Level 5 (L-5) are listed in Table 5.4.

TABLE 5.4
STCDC Toxicity Summary of L-3, L-4, and L-5 v. Time.

| | | Group | Group | Group | Group | Group | Group |
|---|---|---|---|---|---|---|---|
| Dose Groups | | 1 | 2 | 3 | 4 | 5 | 6 |
| Average TOP | 4 hours | 3.4% | 2.6% | 28% | 45% | 63% | 66% |
| per item per | 3 days | N/A | N/A | 9.4% | 30% | 52% | 55% |
| rat | 1 week | N/A | N/A | 3.1% | 5.7% | 11% | 16% |
| | 2 weeks | N/A | N/A | 0.5% | 0.3% | 0.5% | 2.6% |
| Average | 4 hours | 0.0% | 0.0% | 1.0% | 2.9% | 5.7% | 2.3% |
| occurrence | 3 days | N/A | N/A | 0.0% | 1.0% | 0.5% | 3.9% |
| for L-3 per | 1 week | N/A | N/A | 0.0% | 0.0% | 0.0% | 1.3% |
| item per rat, | 2 weeks | N/A | N/A | 0.0% | 0.0% | 0.0% | 0.0% |
| AOL-3 | | | | | | | |
| Average | 4 hours | 0.0% | 0.0% | 0.0% | 1.8% | 2.4% | 6.1% |
| occurrence | 3 days | N/A | N/A | 0.0% | 0.0% | 0.5% | 2.3% |
| for L-4 per | 1 week | N/A | N/A | 0.0% | 0.0% | 0.0% | 0.3% |
| item per rat, | 2 weeks | N/A | N/A | 0.0% | 0.0% | 0.0% | 0.0% |
| AOL-4 | | | | | | | |
| Average | 4 hours | 0.0% | 0.0% | 0.0% | 0.0% | 0.0% | 2.6% |
| occurrence | 3 days | N/A | N/A | 0.0% | 0.0% | 0.0% | 0.0% |
| for L-5 per | 1 week | N/A | N/A | 0.0% | 0.0% | 0.0% | 0.0% |
| item per rat, | 2 weeks | N/A | N/A | 0.0% | 0.0% | 0.0% | 0.0% |
| AOL-5 | | | | | | | |

The STCDC toxicity summary results are detailed in Table 5.4 and FIG. 6A-6C. As shown in these toxicity results, the toxicity of STCDC increases as its concentrations in the formulation increases. High doses of STCDC are associated with high percentages of average TOP. Within Groups 3-6, Group 3, which received the lowest dose of STCDC, had fewer changes than the higher doses of STCDC, particularly after four hours.

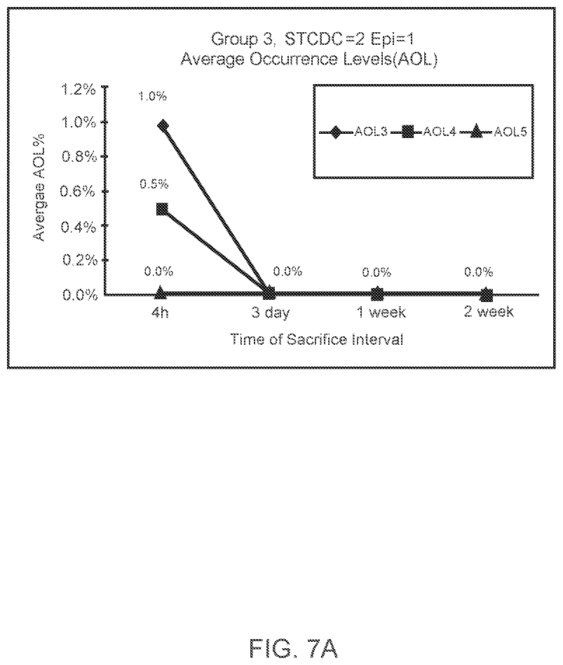

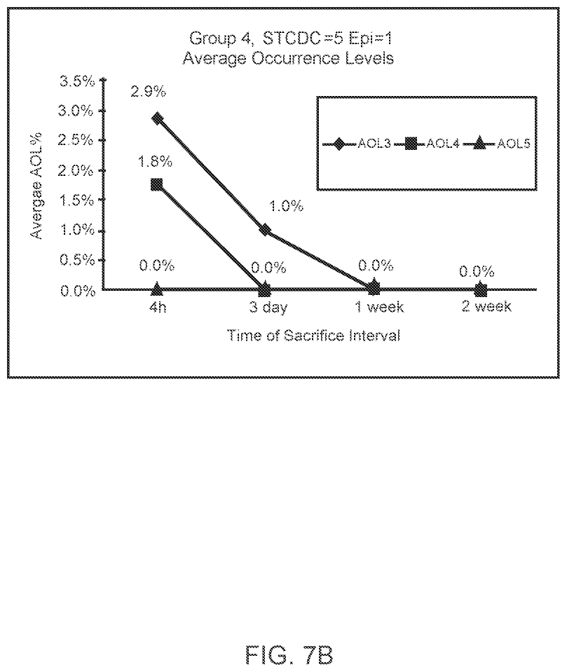

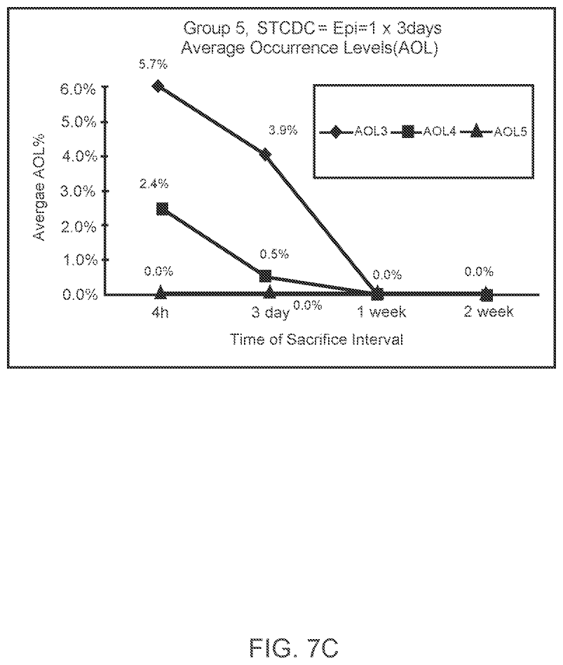

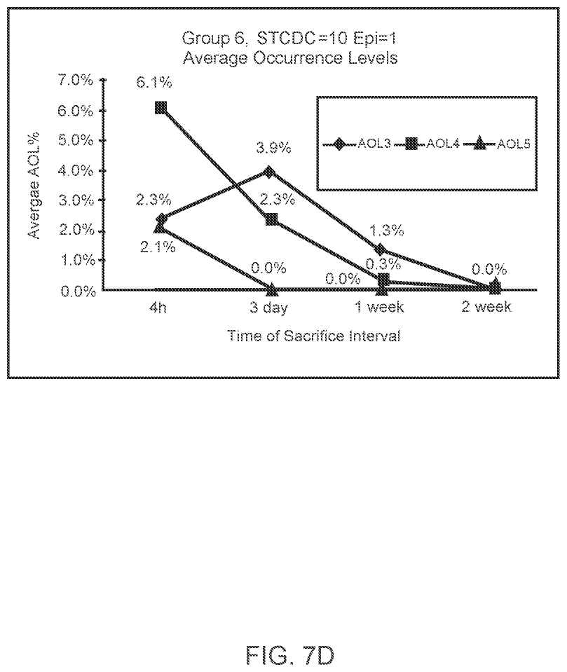

A dose-related increase in the nasal cavity epithelial, inflammatory, and exudative changes was observed after exposure to STCDC and epinephrine. When the average TOP is displayed versus time in FIG. 6D, most observed toxicity items occurred at 4 hours after administration, rapidly decreased over time, and nearly normal at two weeks post-dose. At about 1 week after administration, majority of the observed toxicity items disappeared. A similar decreasing over time trend can be observed for AOL-i (i=3,4,5) as shown in FIG. 7A-7D. Thus, the average TOP percentages are decreasing rapidly from post administration times of 4 hours to two weeks. Therefore, this rapid decrease trend to nearly normal at two weeks post-done suggests that a bile salt, such as STCDC, is safe for clinical applications as an absorption enhancer.

### Example 6-Local Toxicity Study of Bile Salt (STC) as Absorption Enhancer for IN Delivery of Epinephrine

Example 6 describes a human clinical study (Clinical Study A) that provides one or more results that may be achieved using pharmaceutical formulations as disclosed herein. Clinical Study A was a single-center, open-label, active-controlled, single dose ascending study for initial evaluation of pharmacokinetics, pharmacodynamics, safety, and tolerability of test compositions comprising epinephrine and various amounts of bile acid salt enhancer (or lacking bile acid salt enhancer) in adult healthy volunteers, in comparison to the active controls (Epinephrine auto-injector). The study was performed, in part, to identify the clinically useful dose strength(s) for the ensuing pivotal clinical trials. Each intranasal test unit (epinephrine with or without the permeation enhancer, the bile acid salt STC) was provided as a pre-filled, single-dose, intranasal spray comprising pharmaceutical formulation and device components. Each test unit delivered 0.1 mL atomized spray after manual activation. In view of the findings provided here for the test compositions, the same indications as that for Epinephrine injection (EpiPen®) may be used (e.g., for “the emergency treatment of allergic reactions (Type I) including anaphylaxis”).

### Introduction

This study investigated new pharmaceutical formulations (epinephrine with an enhancer, STC) for the delivery of epinephrine intranasally. Intranasal Epinephrine was provided as follows to various participants in different experimental arms of the study. The arms were as follows:
Arm T1: F-a (epinephrine 1.2 mg w/o STC);
Arm T2: F-b (epinephrine 0.6 mg/STC 0.4 mg);
Arm T3: F-c (epinephrine 0.6 mg/STC 0.6 mg);
Arm T4: F-d (epinephrine 0.6 mg/STC 0.8 mg);
Arm T5: F-e (epinephrine 0.9 mg/STC 0.8 mg);
Arm T6: F-f (epinephrine 1.2 mg/STC 0.8 mg); and
Arm T7: F-g (epinephrine 1.2 mg/STC 1.0 mg).

Each of Arms T1-T7 were single dose, intranasal administrations. The control arm was an administered generic version of EpiPen® 0.3 mg (Dose/Route of Administration: Single dose/Intramuscular). STC (sodium taurocholate) worked as the absorption enhancer as epinephrine is a polar drug with low membrane permeability.

The investigators responsible for conducting this study in accordance with the approved protocol were board certified physicians or healthcare professionals who were familiar with the study populations and study medications, and had been trained to handle any untoward events related to medications or procedures. One (1) clinical study center in the United States was used for the execution of Clinical Study A.

### Participant Screening Inclusion and Exclusion Criteria

Subjects were screened for eligibility to enter the study based upon the inclusion and exclusion criteria. Tasks accomplished in the screening visit included: informed consent; evaluation of inclusion/exclusion criteria; and demographics, medical history, physical examination, and pre-study evaluations, including vital signs, ECG, and blood and urinary lab tests (non-fasting). To qualify, subjects had to satisfy all of the following inclusion criteria: Upon review, agreed to participate and sign informed consent; Male and female adults, aged 18-50 years (inclusive); Generally healthy and medically stable, with no clinically significant abnormalities based on physical examination and laboratory tests determined by the investigators; Had normal resting blood pressure (e.g., systolic BP 90-129 mmHg, diastolic BP 60-79 mmHg), normal heart rate (e.g., 55-100 bpm), and normal ECG); Had a body mass index (BMI) of 18.5-29.9 kg/m²; A non-smoker for at least the past 12 months with a pack history of <10 pack years (Pack years=number of cigarettes smoked per day/20*number of years smoked); Female candidates must be >1 year post-menopausal or using a clinically acceptable form of birth control and confirmed by negative urine pregnancy test at Screening; Negative HIV-Ab, HBsAg and HCV-Ab; Negative alcohol test as shown by urine or breathalyzer; Negative drug screening results; With no clinically significant respiratory, cardiovascular, neurological, or other health conditions that, in the opinion of the investigator, may compromise subject safety or interfere with the study conduct and evaluation; No planned major surgery for the entire study period; Not taking any prohibited drugs, including any known COMT and MAO inhibitors or inducers within 5 elimination half-lives prior to and throughout the study; Normosmia was evaluated by The University of Pennsylvania Smell Identification Test (UPSIT) at screening.

Subjects were excluded for any of the following reasons: Evidence or history of any cardiovascular diseases including cardiac dysrhythmia, organic heart disease, coronary artery disease, angina pectoris, myocardial infarction, or hypertension; Evidence or history of any ECG abnormalities, including Long QT syndrome, family history of long QT syndrome, or corrected QT interval (QTcF) >450 ms in men, >470 ms in women; Evidence or history of significant endocrine, neurologic, psychiatric, or other diseases such as hyperthyroidism, diabetes, Parkinson disease, depression, migraine; as well as presence of clinically significant physical or laboratory findings that, in the opinion of the investigator, would put the subject at risk through study participation, or would affect the study analyses if the disease exacerbated during the study; Recent (i.e., within three months) nasal surgery, injury or abnormal nasal anatomy (e.g. nose piercings are not acceptable); Any local nasal disease such as chronic or acute rhinitis, sinusitis or polyps, symptoms of rhinorrhea, epistaxis (e.g., nosebleeds), recent cold or upper respiratory tract infection within 2 weeks of screening; Chronic or current use of all prescription or over-the-counter nasal sprays (e.g., homeopathic remedies); Unable to meet the safety monitoring requirements of this clinical trial or considered by the investigator to be an unsuitable candidate for the study; Women who are pregnant or lactating, or are planning to become pregnant during the study period; Abnormal thyroid function test; Hypersensitivities to epinephrine or other ingredients contained in the formulation; Subject has donated or lost >500 mL of blood within 3 months of screening; or Having been on other clinical drug/device studies in the last 30 days prior to screening.

After satisfying the inclusion/exclusion criteria and lab tests, qualified subjects were assigned a unique subject identification number (Study ID) in sequence at the beginning of the Study Visit. In addition, they were identified by first, (middle) and last initials. The enrollment was confirmed at the beginning of Study Visit-1.

### Cohorts 1 and 2 Design for Study Participants

This study was planned for a single-center, open-label, active-controlled, single ascending dose study in two sequential cohorts of healthy volunteers. Each cohort consisted of a screening visit, five (5) study treatment visits, and a follow-up visit. Subjects received a single dose of generic EpiPen® IM 0.3 mg as the PK reference at the first study visit. Subjects received a single dose of IN epinephrine test composition at IN dosing visit 2-5. The treatment schedule for Cohort 1 and Cohort 2 is found below in Table 6.1.

TABLE 6.1
Dosing amounts in Cohorts 1 and 2 of epinephrine and STC.

| | Cohort 1 | | | | | Cohort 2 | | | | |
|---|---|---|---|---|---|---|---|---|---|---|
| Items | Ra | T1 | T2 | T3 | T4a | Rb | T4b | T5 | T6 | T7 |
| Formulation Code | | F-a | F-b | F-c | F-d | | F-d | F-e | F-f | F-g |
| Delivery Route | IM | IN | IN | IN | IN | IM | IN | IN | IN | IN |
| Epinephrine Dose | 0.3 | 1.2 | 0.6 | 0.6 | 0.6 | 0.3 | 0.6 | 0.9 | 1.2 | 1.2 |
| (mg) | | | | | | | | | | |
| STC Dose (mg) | 0 | | | | | | | | | |
| Label Claim Volume | 0.3 | | | | | | | | | |
| (mL) | | | | | | | | | | |
| Filling Volume (mL) | 0.3 | | | | | | | | | |
| Device/Container | Mylan | Aptar | Aptar | Aptar | Aptar | Mylan | Aptar | Aptar | Aptar | Aptar |
| | | IN | IN | IN | IN | | IN | IN | IN | IN |

A total of 161 subjects were screened for entering this study and 56 subjects passed screening, consented and participated in the study. After satisfying the enrollment criteria, fifty-six (56) healthy volunteers were enrolled in the two (2) sequential cohorts. Each cohort included 28 subjects and investigated up to 4 intranasal (IN) doses and the active comparator by intramuscular (IM), dosed over 5 sessions. For the visits, each cohort had (i) a screening visit (up to 28 days before the first study visit), (ii) five (5) dosing visits, each separated by at least 6 days, and one (1) follow-up visit: 2 weeks (±3 days) after the last Dosing Visit. Subjects received a single dose of generic version of EpiPen® IM 0.3 mg as the PK reference at the first study visit. Subjects also received a single dose of IN test formulation in each of the IN dosing visits 2-5.

The intranasal dosing was performed using a single-use, one-step nasal delivery device. This device is configured to deliver a IN formulation. It can be used in an emergency situation by a non-medical personnel to patients or by patients themselves. During an event (e.g., an allergic reaction), the patient or caregiver can press a small plunger on the bottom of the device to release the drug in a single spray into the nostril. The drug is quickly absorbed via the nasal mucosa. Two dose devices can also be used.

The study was performed following the Helsinki principles and most of the assessments used to measure pharmacokinetics and safety in this protocol are widely used and recognized as reliable. This trial was undertaken in accordance with the current ICH Good Clinical Practice (GCP) guidelines and the current version of the Declaration of Helsinki of the World Medical Association to assure that the rights, safety and well-being of the study subjects are protected. The purpose, methods, potential benefits and risks of the trial were explained to all subjects and subjects were informed that they are free to not participate in the trial and they may withdraw from the study at any time.

In cohort 1, a total of 28, 26, 27, 27, and 28 subjects were treated for Arm T1, T2, T3, T4a, and Ra, respectively. In cohort 2, a total of 26, 25, 24, 24, and 28 subjects were treated for Arm T4b, T5, T6, T7, and Rb, respectively. The mean ages of subjects were 37±9 (range 20-50) years old; and 34±8 (range 20-49) years old for cohort 1 and 2, respectively; the gender profile was about 46% male and 54% female for cohort 1, 43% and 57% in cohort 2; and the race profile showed the majority of the subjects are Caucasian and Black or African American (82% and 89% in cohort 1 and cohort 2, respectively). Each cohort in Study A is a 5-arm crossover study. Therefore, the treatment arms had very limited difference with regards to subject demographics. The demographic data were not significantly different among the study arms.

Additional details regarding Cohort 1 and Cohort 2 are provided in Table 6.2, along with epinephrine doses, the route of administration, the device used to administer the dose, and the volume of formulation delivered by the dosing device.

TABLE 6.2
Study population for Cohorts 1 and 2.

| | | Cohort 1 | | | | | Cohort 2 | | | | |
|---|---|---|---|---|---|---|---|---|---|---|---|
| Notation | Description | T1 | T2 | T3 | T4a | Ra | Rb | T4b | T5 | T6 | T7 |
| | Study Drug | F-a | F-b | F-c | F-d | EpiPen ® | EpiPen ® | F-d | F-e | F-f | F-g |
| | formulation | | | | | | | | | | |
| | code | | | | | | | | | | |
| | Delivery | IN | IN | IN | IN | IM | IM | IN | IN | IN | IN |
| | Route | | | | | | | | | | |
| | Epinephrine | 1.2 | 0.6 | 0.6 | 0.6 | 0.3 | 0.3 | 0.6 | 0.9 | 1.2 | 1.2 |
| | Dose (mg) | | | | | | | | | | |
| | STC Dose (mg) | 0 | 0.4 | 0.6 | 0.8 | 0 | 0 | 0.8 | 0.8 | 0.8 | 1 |
| | Device | Aptar | Aptar | Aptar | Aptar | auto- | auto- | Aptar | Aptar | Aptar | Aptar |
| | | IN | IN | IN | IN | injector | injector | IN | IN | IN | IN |
| | Label Claim | 0.1 | 0.1 | 0.1 | 0.1 | 0.3 | 0.3 | 0.1 | 0.1 | 0.1 | 0.1 |
| | Volume (mL) | | | | | | | | | | |
| N | Target | 25 | | | | | 25 | | | | |
| n₀ | Screened | 161 | | | | | 161 | | | | |
| | Population | | | | | | | | | | |
| n₁ | Enrolled | 28 | | | | | 28 | | | | |
| | Population | | | | | | | | | | |
| | (ITP) a) | | | | | | | | | | |
| n₂ | Treated | 28 | 26 | 27 | 27 | 28 | 28 | 26 | 25 | 24 | 24 |
| | Population | | | | | | | | | | |
| | (TP) b) | | | | | | | | | | |
| n₃ | Evaluable | 28 | 26 | 27 | 26 | 28 | 27 | 26 | 25 | 24 | 24 |
| | Subjects c) | | | | | | | | | | |
| n₄ | PPP c) | 28 | 26 | 27 | 26 | 28 | 25 | 25 | 24 | 23 | 23 |

a) Number of Subjects being Enrolled and assigned subject ID as E5A-Cnn, where nn is the two digits sequential number staring from 01.
b) n2 is the number of subjects received at least one study drug of any treatments. (see Section 5.1.2 of SAP)
c) n3 is the number of evaluable subjects. (see section 5.1.1 of SAP)
d) n4 is the number of subjects in per-protocol population. (see section 5.1.1 of SAP)

The 5-arm crossover in each cohort was intended to minimize inter-individual variations, thus increasing the sensitivity to detect potential differences among study arms. Seven (7) doses of IN formulation (T1-T7) were studied in comparison with the recommended dose of EpiPen® 0.3 mg IM.

If they occurred, protocol deviations were recorded. The nature and reasons for protocol deviations were reviewed, described, and documented before the database was locked for analysis. Per Protocol Population (PPP) is defined as, all subjects who have an evaluable IM injection of Epinephrine (Treatment R), and at least one evaluable visit of four (4) IN treatments (Treatment T). The Treated Population (TP) is defined as all subjects who have been treated with any dose of a study IN formulation. The tolerability and safety evaluations were performed base on treated population (TP). The “intent-to-treat (ITT)” population analysis was performed for the initial safety evaluation. The ITT population is defined as all subjects who have received at least one study drug treatment.

### Treatment Period

For all 5 study visits in each cohort, prior to study drug administration, review/verification was performed to ensure the arrival time and breakfast requirements, baseline measurements, study restrictions, and concomitant medication restrictions, and other health conditions were adequate per the protocol requirements to proceed to the study treatment. Pre-administration evaluations, including baseline vital signs (HR, SBP/DBP), were performed prior to study drug treatment.

Each subject was given IM injection of the reference product (Treatment R) at Study Visit 1 and four (4) single doses of test formulation by IN at Study Visit 2-5 delivery (T1 to T4 in cohort 1 and T4 to T7 in cohort 2, respectively). Table 6.3 summarizes the formulations and active control drug (generic EpiPen®) used in this study.

TABLE 6.3
Test Formulations and the IM Active Control Drug.

| | Comparator | Treatment | | | | | | |
|---|---|---|---|---|---|---|---|---|
| Items | R | T1 | T2 | T3 | T4 | T5 | T6 | T7 |
| E005 Formulation Code | | F-a | F-b | F-c | F-d | F-e | F-f | F-g |
| Delivery Route | IM | IN | IN | IN | IN | IN | IN | IN |
| Epinephrine Dose (mg) | 0.3 | 1.2 | 0.6 | 0.6 | 0.6 | 0.9 | 1.2 | 1.2 |
| STC Dose (mg) | 0 | 0 | 0.4 | 0.6 | 0.8 | 0.8 | 0.8 | 1 |
| Label Claim Volume | 0.3 | 0.1 | 0.1 | 0.1 | 0.1 | 0.1 | 0.1 | 0.1 |
| (mL) | | | | | | | | |
| Filing Volume (mL) | 0.3 | 0.3 | 0.125 | 0.125 | 0.125 | 0.125 | 0.125 | 0.125 |
| Device/Container | Mylan | Aptar IN | Aptar IN | Aptar IN | Aptar IN | Aptar IN | Aptar IN | Aptar IN |
| Formulation (mg/mL) | | | | | | | | |
| L-Epinephrine | 1 | 12 | 6 | 6 | 6 | 9 | 12 | 12 |
| Sodium Taurocholate | 0 | 0 | 4 | 6 | 8 | 8 | 8 | 10 |
| (STC) | | | | | | | | |
| Citric Acid | 0 | 4 | 4 | 4 | 4 | 4 | 4 | 4 |
| (monohydrate) | | | | | | | | |
| Sodium Citrate | 0 | 8 | 8 | 8 | 8 | 8 | 8 | 8 |
| (dihydrate) | | | | | | | | |
| Chlorobutanol | 0 | 5.5 | 5.5 | 5.5 | 5.5 | 5.5 | 5.5 | 5.5 |
| (hemihydrate) | | | | | | | | |
| NaCl | 6 | 2.5 | 3 | 3 | 2.5 | 2.5 | 2.2 | 2 |
| EDTA (dihydrate) | 0 | 0.02 | 0.02 | 0.02 | 0.02 | 0.02 | 0.02 | 0.02 |
| Sodium metabisulfite | 1.67 | 0.3 | 0.3 | 0.3 | 0.3 | 0.3 | 0.3 | 0.3 |
| pH | 3.8 | 3.8 | 3.8 | 3.8 | 3.8 | 3.8 | 3.8 | 3.8 |
| Osmolarity (mOsmol) | 237 | 248 | 265 | 265 | 248 | 248 | 238 | 231 |

The absorption enhancer STC shows acceptable toxicological profile and desirable enhancement effects between 4-10 mg/mL in the rat model. In this first in-human trial, test formulations were tested in seven (7) doses (T1 to T7). As shown in Table 6.3, the starting dose of IN treatment for the first cohort was the T1 (F-a formulation: 1.2 mg Epinephrine without absorption enhancer STC). Subjects received a single dose of generic EpiPen® IM 0.3 mg as the PK reference at the study visit 1. Subjects further received a single dose of IN test formulation at IN dosing visit 2-5. The decision to proceed to the next dose level was made by the study Dose Escalating Committee (DEC) that is composed by the Statistician, Investigator and Medical Monitor. The DEC reviewed the safety, tolerability and preliminary pharmacokinetic, pharmacodynamic data obtained from at least 12 subjects at the current dose level to make the recommendation. There were 5 DEC meetings (T2 to T3, T3 to T4, T4 to T5, T5 to T6, and T6 to T7) in this study, as showed in Table 6.4.

The following procedures/guidelines were used for IM injection: (1) Subject should be lying down; (2) Drug was injected in the middle of the outer thigh (upper leg); (3) Clean the injection site with alcohol swab in a circular motion; (4) The medical professional held the leg firmly in place, then place the orange tip against the middle of the outer thigh (upper leg) at a right angle (perpendicular) to the thigh. (5) Swing and push the auto-injector firmly until it “clicks.” The click signals that the injection has started. (6) Hold firmly in place for 3 seconds. Time 0 of PK sampling begins at this point for IM injection. (7) Remove the auto-injector from the thigh. The orange tip will extend to cover the needle. (8) Massage the injection area for 10 seconds. (9) The injection site was observed for any medication leakage for approximately 30 seconds after the dosing is completed. If there is observed leakage, the samples were not collected and the study visit was early terminated.

The following procedures/guidelines were used for IN administration: (1) Subjects were instructed to blow their nose gently before the drug administration; (2) Subjects lay flat with their head/neck straight. The designated doser placed the tip of the IN formulation device into nostril and quickly depressed the base of vial until the contents are delivered. Time 0 began at this point for IN Treatment T1-T7. (3) Alternative nostrils were between treatments to allow for at least 2 weeks between exposure to STC for each nostril to allow for evaluation of any adverse drug events; (4) Following the drug administration, subjects were instructed breathe gently through the mouth for 5 to 10 seconds and not to swallow; (5) Once the IN delivery was completed, subjects laid flat and remained in the supine position for 30 minutes.

Pharmacokinetics of the epinephrine at each visit was evaluated at baseline, and at 1, 3, 5, 7, 10, 15, 20, 30, 45, 60, 90, 120, 240, and 360 minutes post-dose for each dosing visit. Upon completion of Visit-5, subjects were subjected to a follow-up safety evaluation. Follow-up evaluations were performed at 2 weeks (±3 days) after the last Dosing Visit. Those subjects who withdrew or were withdrawn prematurely were subject to an early termination safety evaluation.

### Pharmacokinetic Measurements

At Study Visits 1 to 5, PK blood samples were collected and plasma was isolated for analyzing concentrations of epinephrine. A total of 15 blood samples were drawn from each subject at each Study Visit as indicated in Table 6.4. A total volume of approximately 75 mL blood was collected for PK analysis at each study visit. A saline catheter flush was performed between each blood sample collection. At each sampling point, the first 1.0 mL portion of the blood (or saline flush) was collected into a disposable tube and discarded; Time 0 started after administration of Treatment T into one nostril was completed; for IM injections (Treatment R), Time 0 began at the end of injection (holding time 3 seconds); At each PK sampling point, blood samples (˜5 mL), were collected in ice-chilled K₂EDTA sample tubes; Immediately after the sample collection, the phlebotomist gently inverted the tube approximately 8-10 times to mix it well; If a tube broke, a new sample could have been collected if it was within the allowed time window (Table 6.4); and Plasma isolation (i.e., centrifugation of the blood samples) started within 20 minutes of blood collection, sample tubes were chilled on ice after collection. Blood samples were centrifuged at 2-8° C., 2,000-3,000 g for 20 minutes for plasma isolation. Isolated plasma was transferred into two (2) 2 mL cryo vials so that the vial contains approximately 1 mL plasma per sample obtained. The plasma sample vials were frozen on dry ice within 60 minutes and then stored in a freezer at −20° C. or lower until analysis.

TABLE 6.4
PK Blood Sampling Schedule (~5 mL per sample)

| | | | Sample | | | | | Sample |
|---|---|---|---|---|---|---|---|---|
| Seq. # | Scheduling | Time window | No., “XX” | # | Scheduling | | Time Window | No., “XX” |
| 1 | Baseline | within 30 min | 01 | 9 | 30 | min | ±2 min | 09 |
| | | pre dosing | | 10 | 45 | min | | 10 |
| | | | | 11 | 60 | min | | 11 |
| 2 | 1 min | ±1 min | 02 | 12 | 90 | min | ±5 min | 12 |
| 3 | 3 min | | 03 | 13 | 2 | h | | 13 |
| 4 | 5 min | | 04 | 14 | 4 | h | | 14 |
| 5 | 7 min | | 05 | | | | | |
| 6 | 10 min | | 06 | | | | | |
| 7 | 15 min | | 07 | | | | | |
| 8 | 20 min | ±2 min | 08 | 15 | 6 | h | ±10 min | 15 |

| | |
|---|---|
| Total | 15(=75(mL) |

Vital Signs: Readings for heart rate (HR), systolic blood pressure (SBP), diastolic blood pressure (DBP), and respiratory rate (RR) were collected. The pharmacodynamic collection scheme aligned to the pharmacokinetic (PK) sampling scheme.

### Study Objectives

The main objective of this study was to initially investigate pharmacokinetics, pharmacodynamics, and safety/tolerability profiles of the proposed IN epinephrine product, and to identify the optimum dose strength. Investigation into the comparability of IN and IM administration routes, partial AUC_{0-t*}, AUC_{0-t*}, and Cₘₐₓ were evaluated as primary PK endpoints. Primary endpoints included:
AUC_{0-t*}, (where t*=tₘₐₓ^{IM}, of Treatment R), defined as area under the curve (AUC) in the plot of plasma Epinephrine concentration versus time from time 0 to the tₘₐₓ of Epinephrine delivered by IM of EpiPen®;
AUC_{0-t*}, where t′ is defined as the time at which the plasma epinephrine concentration of a given IN treatment first reaches the peak plasma epinephrine concentration of the IM treatment of EpiPen® (Cₘₐₓ^{IM}). Namely, t′ satisfies the following equation:

C′^{IN}(t′)=Cₘₐₓ^{IM},where t′<tₘₐₓ^{IM}

AUC_{0-t*} and t′ characterize the pharmacokinetics and the time of onset of test formulation.
Cₘₐₓ was defined as the peak plasma epinephrine concentration.

Secondary pharmacokinetic endpoints were also measured. The secondary pharmacokinetic endpoints included:
tₘₐₓ, defined as the time, at which the peak plasma epinephrine concentration (Cₘₐₓ) is observed.
Partial AUC including AUC₀₋₅, AUC_{0-10′}, AUC_{0-30′};
   where AUC₀₋₂₈, defined as AUC of plasma epinephrine concentrations for time 0 to infinity; AUC₀₋₆ₕᵣₛ, defined as AUC in the plot of plasma epinephrine concentrations versus time from time 0 to the last sampling point; and C^{IN} (t*), defined as epinephrine concentrations of IN delivery at the tₘₐₓ of IM of treatment R. Relative bioavailability (RBA) of IN epinephrine treatments versus IM epinephrine treatments was also determined.

Additional pharmacokinetic endpoints were also measured for the enhancing agent, STC. The additional endpoints for STC included:
AUC₀₋₆ₕᵣs and AUC_{0-∞}; and
Cₘₐₓ.

### Pharmacodynamic and Safety Evaluations

Pharmacodynamic measurements and Safety Evaluations were also made. Four (4) pharmacodynamic parameters: systolic blood pressure (SBP), diastolic blood pressure (DBP), heart rate (HR), and respiratory rate (RR) were measured aligned to the PK sampling scheme. The changes AVS and relatively changes A % VS to the same day baseline were evaluated.

Safety of the study treatments was assessed by tabulations of adverse events and was presented with descriptive statistics at baseline and each treatment arm. Adverse events (AEs) were summarized by treatment group. Incidents and incidence rate (IR) of each type of AE observed was compared between treatment arms.

Adverse events (AE) were classified on the basis of MedDRA terminology and summarized for each treatment arm. AE incidence rates were summarized by severity, relationship to study drug, seriousness and subject outcome. All information pertaining to adverse events noted during the study was listed for subject, AE code, onset time and date, phase, severity, action taken, relationship to study drug, subject outcome, time resolved and date, seriousness and causing withdrawal or not. Safety parameters, such as vital signs (blood pressure [SBP/DBP] heart rate [HR], and respiratory rate [RR]-measurement of vital signs aligned with the PK sampling schema and in follow-up visit), ECG readings ECG (Routine and QT/QTc analysis-measured pre-dose and 10 min and 30 min, 6 hour post-dose, and in follow-up visit), physical examination findings (pre-dose and 10 min and 30 min, 6 hour post-dose, and in follow-up visit), and clinical laboratory results, at end of study were compared with the baseline data. Additionally, post-dose vital signs and ECG (Routine and QT/QTc analysis) readings for each study visit were evaluated. The n, mean and standard deviation of SBP/DBP, HR, RR, and ECG readings were summarized per treatment arm by time of testing. The frequency count and percentage of any significant changes (end of study vs. baseline at screening) in physical exam, ECG and Lab results were reported. Any early termination would be listed with primary reasons per treatment arm as part of safety assessment.

Safety parameters, e.g., vital signs, ECG, physical examinations, CBC, serum comprehensive metabolic panel, and urinalysis, and adverse events were monitored, documented, assessed, and summarized. The severity of adverse events was evaluated using the FDA guidance

### Toxicity Grading Scale for Healthy Adult and Adolescent Volunteers Enrolled in Preventive Vaccine Clinical Trials.

### Intranasal Tolerability and Olfactory Function Evaluation

Several nasal tolerability and olfactory functions tests were also performed. Intranasal tolerability and olfactory function was monitored through the study using Nasal and Oropharyngeal Mucosal Examination (NOME); Subjects Self-Reported Nasal Symptoms (SRNS), and The University of Pennsylvania Smell Identification Test (UPSIT).

NOME was performed by an ENT specialist or other qualified medical professional. The examiner evaluated and recorded any abnormalities including 1) nasal irritation; 2) mucosal erythema; 3) mucosal edema; 4) nasal discharge; 5) mucosal crusting; and 6) mucosal epistaxis. Nasal floor, septum, and turbinates; as well as compartments within the oropharyngeal passage including soft palate, tonsil/tonsillar fossa, base of tongue, and the posterior pharyngeal wall were evaluated. For SRNS, all participants were instructed to rate all four (4) symptoms in the Total Nasal Symptom Score (TNSS): 1) rhinorrhea (runny nose); 2) nasal congestion; 3) nasal itching; and 4) sneezing. Given the several test formulations contain a STC, two (2) additional symptoms 5) nasal discomfort; and 6) facial pain/pressure were also evaluated. The NOME and SRNS were collected at the: screening visit; pre-dose, 1 hour (±15 min), and 6 hours (±20 min) post-dose in IN dosing visits 2-5; and; follow-up visit.

UPSIT evaluations were performed to assess subjects' olfactory function. The UPSITs were performed at: screening visit; pre-dose, and 6 hours (+20 min) post-dose in IN dosing visits 2-5; and; follow-up visit.

### Results of Pharmacokinetic Analysis for Epinephrine

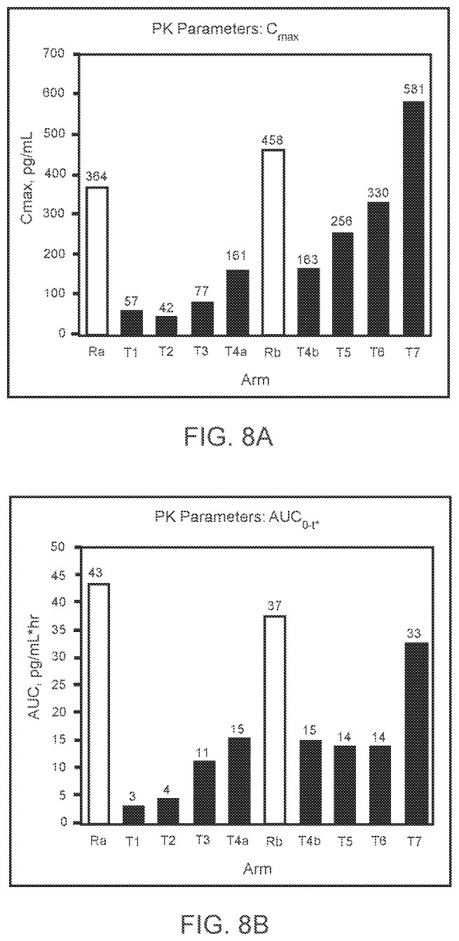

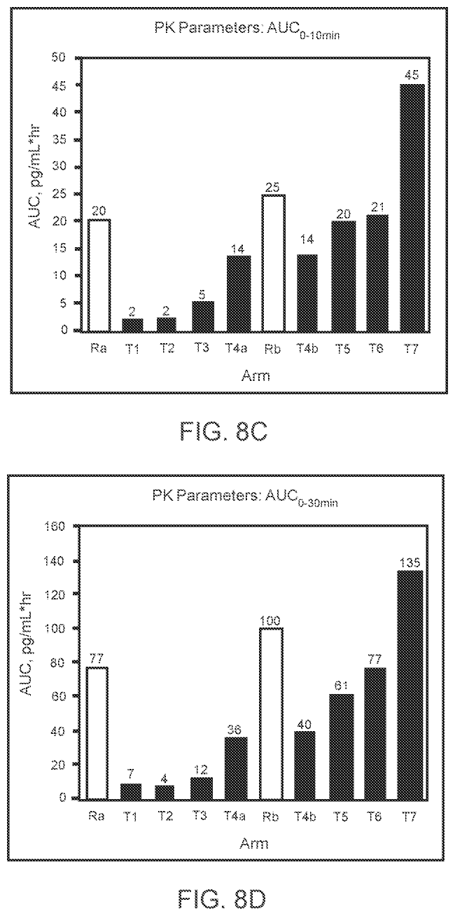

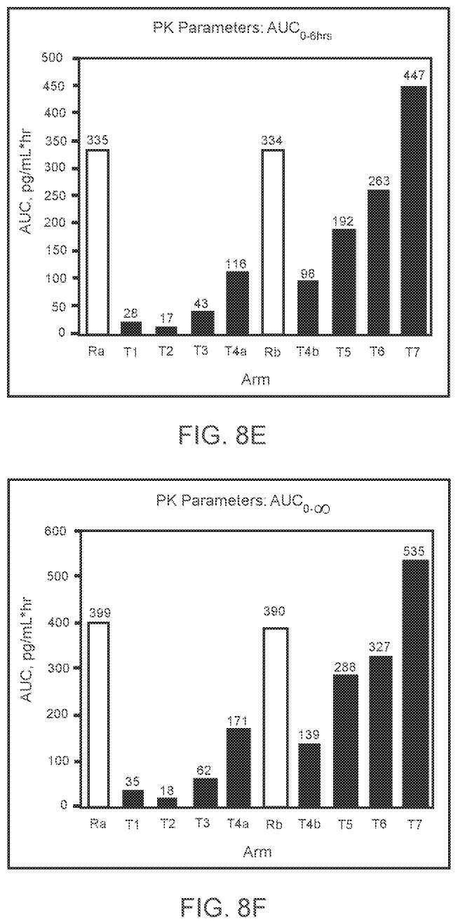

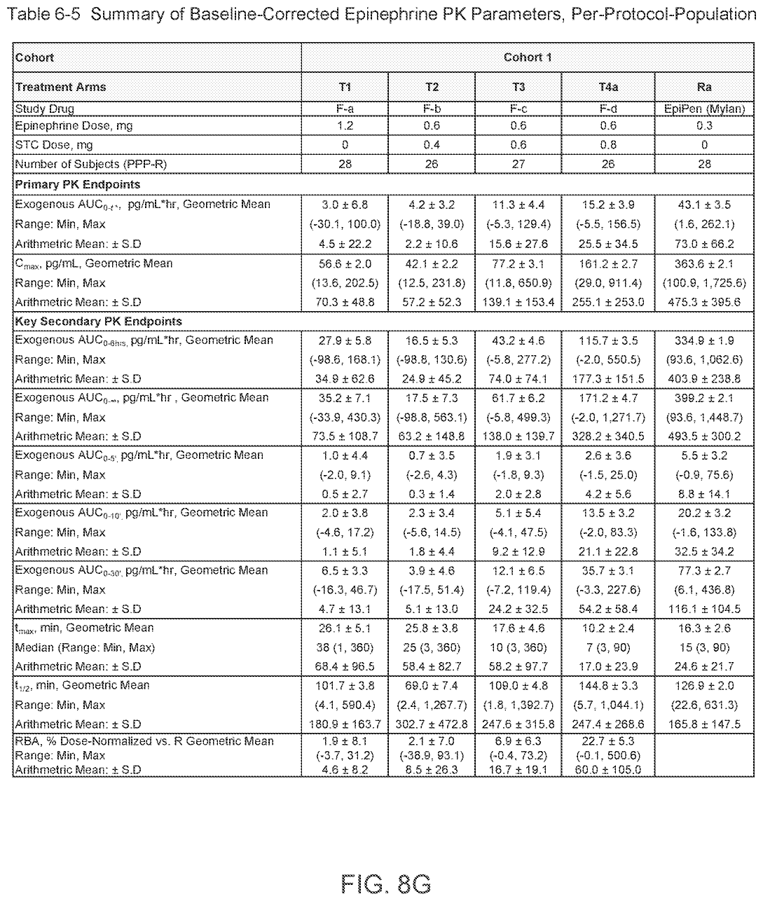

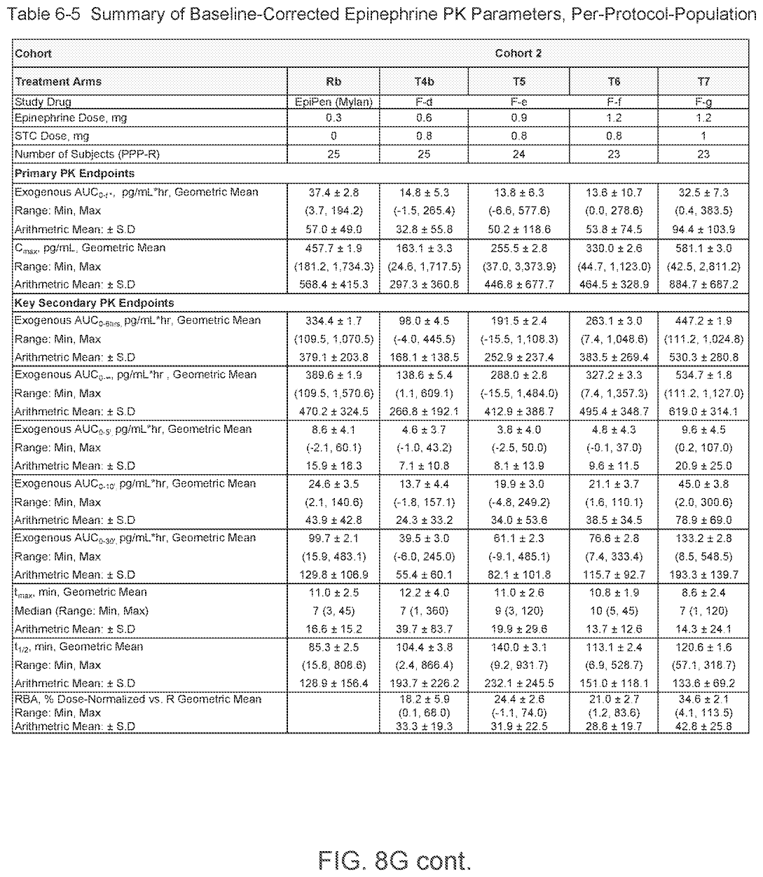

The clinical pharmacokinetic results, including product information, are summarized in FIGS. 8A-8F. The geometric mean of Cₘₐₓ, AUC_{0-t*}, AUC_{0-10 min}, AUC_{0-30 min}, AUC₀₋₆ₕᵣ, and AUC_{0-inf} for 10 treatments (8 IN and 2 IM treatments), are presented in FIG. 8G (Table 6.5).

### Primary Endpoint Evaluations

In cohort 1, the geometric mean of partial AUC_{0-t*} (t*=tₘₐₓ^{IM}, , 0.3 mg) was 43.1 pg/mL*hr for the reference IM treatment Ra. The geometric means of AUC_{0-t*} were 3.0, 4.2, 11.3, and 15.2 pg/mL*hr for the Treatment T1 (epinephrine 1.2 mg without STC) and T2, T3, T4a (epinephrine 0.6 mg with STC 0.4 mg, 0.6 mg, and 0.8 mg), respectively. In cohort 2, the geometric mean of partial AUC_{0-t*}(t*=tₘₐₓ^{IM}, 0.3 mg) was 37.4 pg/mL*hr for the reference IM treatment Rb. The geometric means of AUC_{0-t*} were 14.8, 13.8, 13.6, and 32.5 pg/mL*hr for the Treatment T4b, T5, T6 (epinephrine 0.6 mg, 0.9 mg, and 1.2 mg with STC 0.8 mg) and T7 (epinephrine 1.2 mg with STC 1.0 mg), respectively.

In cohort 1, the geometric mean of Cₘₐₓ was 363.6 pg/mL for the reference IM treatment Ra. The geometric means of Cₘₐₓ were 56.6, 42.1, 77.2, and 161.2 pg/mL for the Treatment T1 (epinephrine 1.2 mg without STC) and T2, T3, T4a (epinephrine 0.6 mg with STC 0.4 mg, 0.6 mg, and 0.8 mg), respectively. In cohort 2, the geometric mean of Cₘₐₓ was 457.7 pg/mL for the reference IM treatment Rb. The geometric means of Cₘₐₓ were 163.1, 255.5, 330.0, and 581.1 pg/mL for the Treatment T4b, T5, T6 (epinephrine 0.6 mg, 0.9 mg, and 1.2 mg with STC 0.8 mg) and T7 (epinephrine 1.2 mg with STC 1.0 mg), respectively.

### Secondary Endpoint Evaluations

In cohort 1, the geometric mean of AUC₀₋₆ₕᵣ was 334.9 pg/mL*hr for the reference IM treatment Ra. The geometric means of AUC₀₋₆ₕᵣ were 27.9, 16.5, 43.2, and 115.7 pg/mL*hr for the Treatment T1 (epinephrine 1.2 mg without STC) and T2, T3, T4a (epinephrine 0.6 mg with STC 0.4 mg, 0.6 mg, and 0.8 mg), respectively.

In cohort 2, the geometric mean of AUC₀₋₆ₕᵣ was 334.4 pg/mL*hr for the reference IM treatment Rb. The geometric means of AUC₀₋₆ₕᵣ were 98.0, 191.5, 263.1, and 447.2 pg/mL*hr for the Treatment T4b, T5, T6 (epinephrine 0.6 mg, 0.9 mg, and 1.2 mg with STC 0.8 mg) and T7 (epinephrine 1.2 mg with STC 1.0 mg), respectively.

In cohort 1, the geometric mean of AUC_{0-30′} was 77.3 pg/mL*hr for the reference IM treatment Ra. The geometric means of AUC₀₋₃₀ were 6.5, 3.9, 12.1, and 35.7 pg/mL*hr for the Treatment T1 (epinephrine 1.2 mg without STC) and T2, T3, T4a (epinephrine 0.6 mg with STC 0.4 mg, 0.6 mg, and 0.8 mg), respectively.

In cohort 2, the geometric mean of AUC_{0-30′} was 99.7 pg/mL*hr for the reference IM treatment Rb. The geometric means of AUC_{0-30′} were 39.5, 61.1, 76.6, and 133.2 pg/mL*hr for the Treatment T4b, T5, T6 (epinephrine 0.6 mg, 0.9 mg, and 1.2 mg with STC 0.8 mg) and T7 (epinephrine 1.2 mg with STC 1.0 mg), respectively.

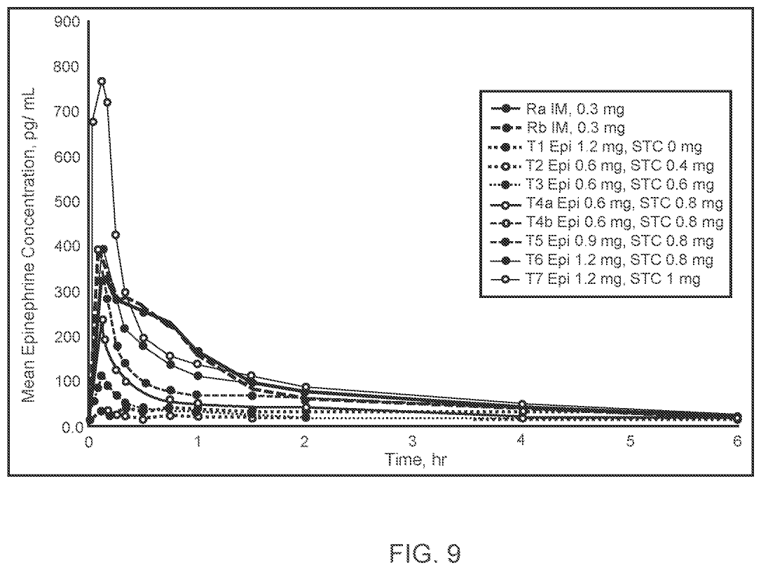

FIGS. 8A-8F provide graphical representations of major PK parameter is of STC Enhancement Effect on Epinephrine. The geometric mean of Cₘₐₓ, AUC_{0-t*}, AUC_{0-10 min}, AUC_{0-30 min}, AUC₀₋₆ₕᵣ, and AUC_{0-inf} for 10 treatments (8 IN and 2 IM treatments), are presented in FIGS. 8A-8F. FIG. 9 illustrates baseline-adjusted plasma epinephrine concentration vs. time curve in all treatment arms for the study (Plasma Epinephrine Concentration vs Time Curves in all Evaluable Population).

In cohort 1, the median tₘₐₓ was 15 minutes for the reference IM treatment Ra. The median tₘₐₓ were 38, 25, 10, and 7 minutes for the Treatment T1 (epinephrine 1.2 mg without STC) and T2, T3, T4a (epinephrine 0.6 mg with STC 0.4 mg, 0.6 mg, and 0.8 mg), respectively.

In cohort 2, the median tₘₐₓ was 7 minutes for the reference IM treatment Rb. The geometric mean of tₘₐₓ were 7, 9, 10, and 7 minutes for the Treatment T4b, T5, T6 (epinephrine 0.6 mg, 0.9 mg, and 1.2 mg with STC 0.8 mg) and T7 (epinephrine 1.2 mg with STC 1.0 mg), respectively.

In cohort 1, the geometric means of Dose Normalized RBA of IN treatments were 1.9, 2.1, 6.9, and 22.7 for T1, T2, T3, and T4a compared to reference treatment Ra, respectively. In cohort 2, the geometric means of Dose Normalized RBA of IN treatments were 18.2, 24.4, 21.0, and 34.6 for T4b, T5, T6, and T7 compared to reference treatment Rb, respectively.

Plasma Epinephrine Concentration vs. Time Profile

Table 6.6 summarizes numbers and proportions of subjects with plasma epinephrine concentration (baseline-unadjusted) ≥100 pg/mL and >200 pg/mL within 10 minutes, 30 minutes, and 1 hour.

TABLE 6.6
Plasma Epinephrine Concentration.

| Cohort | Highest C Cohort-1 | | | | | Highest C for Cohort-2 | | | | |
|---|---|---|---|---|---|---|---|---|---|---|
| Treatment | Ra | T1 | T2 | T3 | T4a | Rb | T4b | T5 | T6 | T7 |
| Epi | 0.3 | 1.2 | 0.6 | 0.6 | 0.6 | 0.3 | 0.6 | 0.9 | 1.2 | 1.2 |
| STC | 0 | 0 | 0.4 | 0.6 | 0.8 | 0 | 0.8 | 0.8 | 0.8 | 1 |
| n= | 28 | 28 | 26 | 27 | 26 | 27 | 26 | 25 | 24 | 24 |
| # of Subjects, with C >= 100 pg/mL | | | | | | | | | | |
| in 10 min | 27 | 3 | 3 | 9 | 18 | 23 | 17 | 21 | 18 | 20 |
| in 30 min | 28 | 4 | 3 | 10 | 18 | 27 | 17 | 22 | 19 | 22 |
| in 60 min | 28 | 5 | 3 | 10 | 18 | 27 | 17 | 22 | 20 | 22 |
| in all time (Cmax) | 28 | 6 | 3 | 10 | 18 | 27 | 17 | 22 | 20 | 22 |
| # of Subjects, with C >= 200 pg/mL | | | | | | | | | | |
| in 10 min | 19 | 1 | 1 | 9 | 11 | 18 | 13 | 14 | 15 | 19 |
| in 30 min | 19 | 1 | 1 | 9 | 11 | 23 | 13 | 15 | 16 | 19 |
| in 60 min | 21 | 1 | 1 | 9 | 11 | 24 | 13 | 15 | 16 | 19 |
| in all time (Cmax) | 21 | 1 | 1 | 9 | 11 | 24 | 13 | 15 | 16 | 19 |
| % of Subjects, with C >= 100 pg/mL | | | | | | | | | | |
| in 10 min | 96.4% | 10.7% | 11.5% | 33.3% | 69.2% | 85.2% | 65.4% | 84.0% | 75.0% | 83.3% |
| in 30 min | 100.0% | 14.3% | 11.5% | 37.0% | 69.2% | 100.0% | 65.4% | 88.0% | 79.2% | 91.7% |
| in 60 min | 100.0% | 17.9% | 11.5% | 37.0% | 69.2% | 100.0% | 65.4% | 88.0% | 83.3% | 91.7% |
| in all time (Cmax) | 100.0% | 21.4% | 11.5% | 37.0% | 69.2% | 100.0% | 65.4% | 88.0% | 83.3% | 91.7% |
| % of Subjects, with C >= 200 pg/mL | | | | | | | | | | |
| in 10 min | 67.9% | 3.6% | 3.8% | 33.3% | 42.3% | 66.7% | 50.0% | 56.0% | 62.5% | 79.2% |
| in 30 min | 67.9% | 3.6% | 3.8% | 33.3% | 42.3% | 85.2% | 50.0% | 60.0% | 66.7% | 79.2% |
| in 60 min | 75.0% | 3.6% | 3.8% | 33.3% | 42.3% | 88.9% | 50.0% | 60.0% | 66.7% | 79.2% |
| in all time (Cmax) | 75.0% | 3.6% | 3.8% | 33.3% | 42.3% | 88.9% | 50.0% | 60.0% | 66.7% | 79.2% |

| | |
|---|---|
| Reference | SAR Table 2226 |

As demonstrated in Table 6.6, Treatment T7 had a comparable PK profile to the reference Ra and Rb. Specifically, the proportions of subjects who reached a plasma epinephrine concentration of 100 pg/mL or greater for Ra, Rb, and T7 were: 96.4%, 85.2%, and 83.3%, within 10 minutes; 100%, 100%, and 92%, within 30 and 60 minutes, respectively. The proportions of subjects who reached a plasma epinephrine concentration of 200 pg/mL or greater for Ra, Rb, and T7 were: 67.90%, 66.7%, and 79.200 within 10 minutes; 67.9%, 85.2%, and 79.2% within 30 minutes; 75.0%, 88.9%, and 79.2%, within 60 minutes, respectively.

Overall, the epinephrine PK profile could be summarized as follows: Baseline levels of plasma epinephrine were detected in both IN and IM treatments. Over the dose range investigated, Cₘₐₓ and AUCs increased with increases either in STC dose (Treatment T2, T3, and T4a) or in epinephrine dose (Treatment T4b, T5, and T6). IN delivery of test formulation resulted in higher maximum concentration (Cₘₐₓ) of epinephrine. The epinephrine concentration declined faster in IN delivery. Higher STC doses were associated with more rapid epinephrine absorption (tₘₐₓ). Treatment T7 (epinephrine 1.2 mg with STC 1.0 mg) appeared to have a comparable PK profile to the reference, as demonstrated by proportions of subjects with plasma epinephrine concentration greater than specific thresholds (100 pg/mL and 200 pg/mL) within 1 hour. Enhancement Factor

The DN-RBA and R(S) for Example 6 are shown below in Table 6.7.

Enhancement Factor was also calculated as shown in Table 6.8. D_{IM}: 1 mg/mL; (AUC_{0-30min)IM}: 88.5; (AUC_{0-∞})_{IM}: 394.5; (Cₘₐₓ)_{IM}t: 411; ^{R̅}(0) (T1) (%): 0.85.

TABLE 6.7
DN-RBA and R(S) for Clinical Studies.

| | D_{IM} | D_{IN} | DN-RBA | | | R(S) | | |
|---|---|---|---|---|---|---|---|---|
| Group | (mg/mL) | (mg/mL) | AUC₀₋₃₀ₘᵢₙ | AUC_{0-∞} | Cₘₐₓ | R(AUC₀₋₃₀ₘᵢₙ) | R(AUC_{0-∞}) | R(Cₘₐₓ) |
| T1 | 1 | 12 | 7 | 35 | 57 | 0.7 | 0.7 | 1.2 |
| T2 | 1 | 6 | 4 | 18 | 42 | 0.8 | 0.8 | 1.7 |
| T3 | 1 | 6 | 12 | 62 | 77 | 0.7 | 0.8 | 0.9 |
| T4 | 1 | 6 | 38 | 155 | 162 | 7.2 | 6.5 | 6.6 |
| T5 | 1 | 9 | 61 | 288 | 256 | 2.3 | 2.4 | 2.1 |
| T6 | 1 | 12 | 72 | 327 | 330 | 6.8 | 6.9 | 6.7 |
| T7 | 1 | 12 | 133 | 535 | 581 | 12.5 | 11.3 | 11.8 |

TABLE 6.8
Enhancement Factor From Clinical Studies.

| Group | Mean R̅(S)(%) | EF |
|---|---|---|
| T1 [R(0)] | 0.85 | 1.0 |
| T2 | 1.07 | 1.26 |
| T3 | 0.80 | 0.94 |
| T4 | 6.76 | 7.94 |
| T5 | 2.27 | 2.67 |
| T6 | 6.79 | 7.98 |
| T7 | 11.87 | 13.94 |

### Results of Pharmacokinetic Analysis of STC

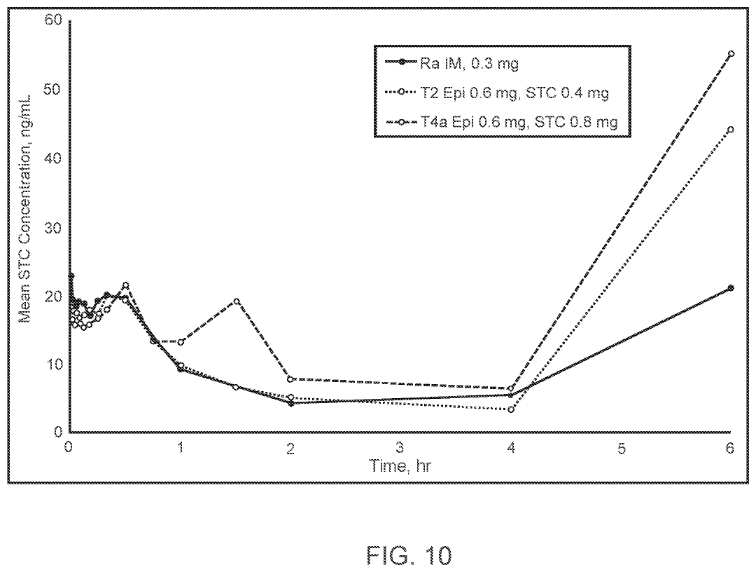

The absorption enhancer STC levels were assessed in IN Treatment T2, T4a and reference IM Treatment Ra in cohort 1. FIG. 10 illustrates plasma STC concentration vs time curve. The STC PK profile could be summarized as follows: A baseline level of plasma STC was approximately 20 pg/mL; STC concentration declined to approximately 50% f baseline level during 2-4 hrs post-dose; STC level increased significantly from 4 hrs post-dose, when subjects started to eat their meal, and the high physiological post-meal STC level indicates that the STC level is safe post-dosing (0-4 hrs); The curves for T2 and T4a (subject are crossover in Cohort-1) were comparable; The STC PK profile indicates the absorption of STC from test formulations is negligible.

### Results of Pharmacodynamic Parameters

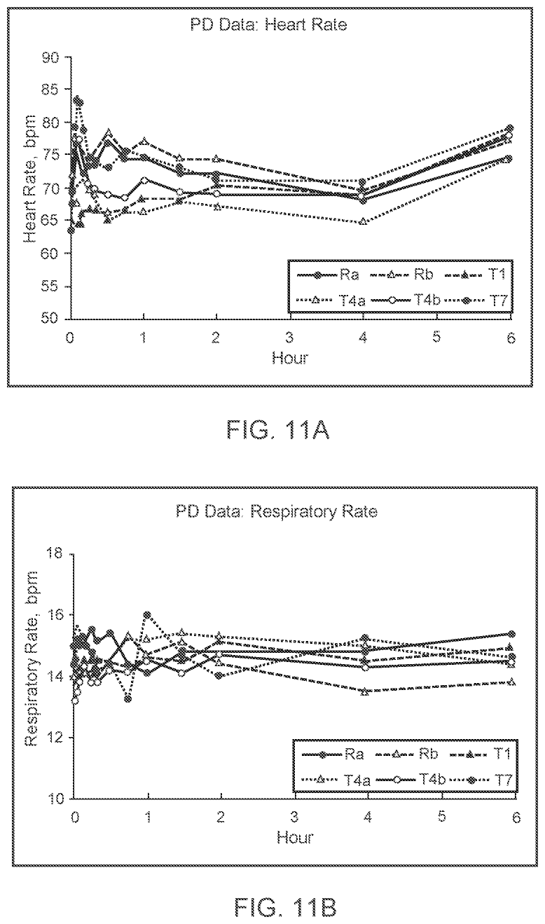

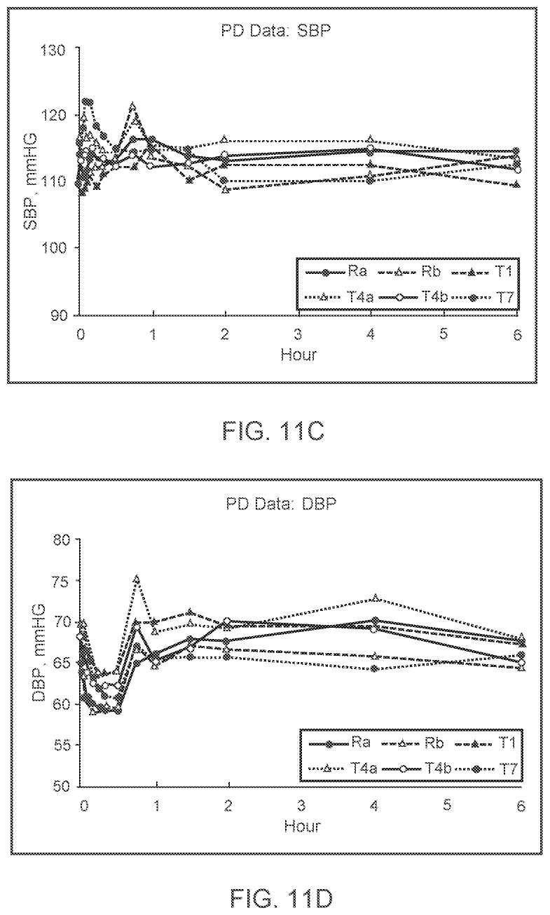

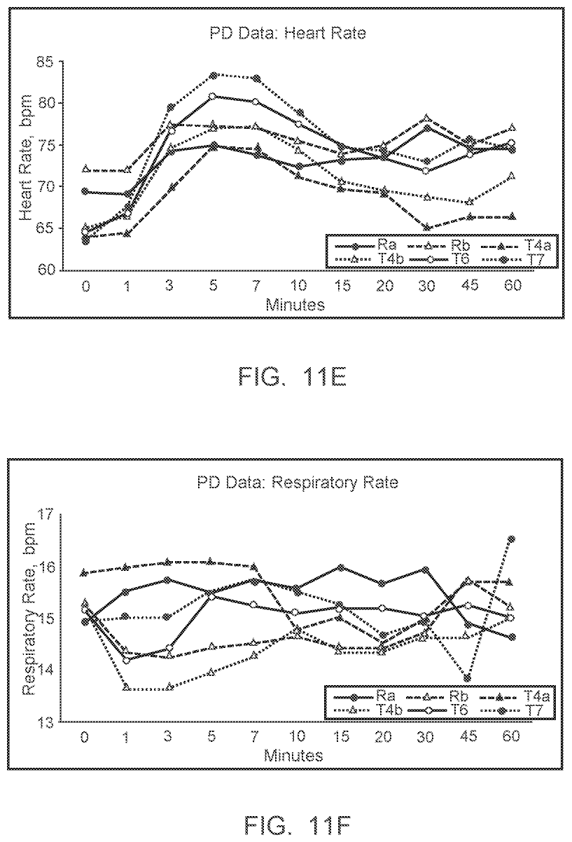

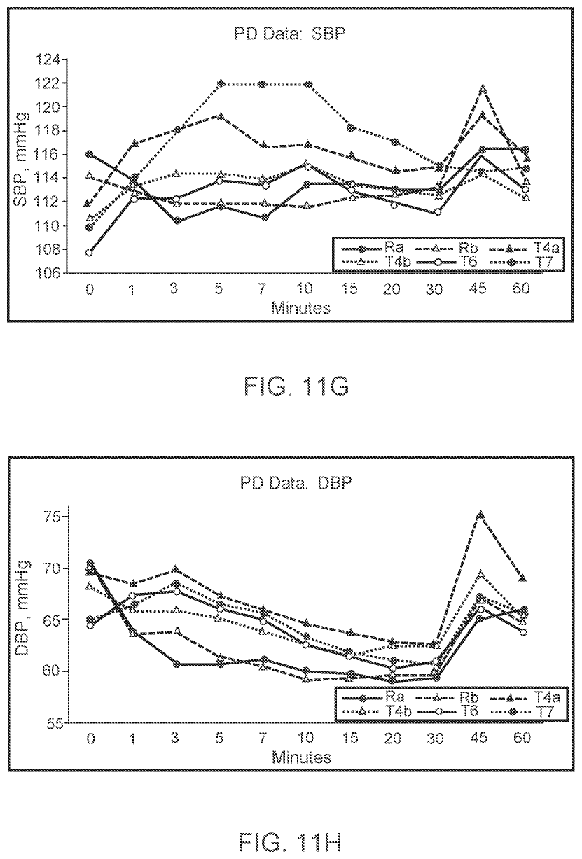

Four (4) Pharmacodynamic parameters: systolic blood pressure (SBP), diastolic blood pressure (DBP), heart rate (HR), and respiratory rate (RR) were measured concurrent with the PK sampling scheme. A total of 15 measurements were taken at baseline, and 1, 3, 5, 7, 10, 15, 20, 30, 45, 60, 90, 120, 240, and 360 minutes post-dose for each parameter. The following results were observed in Study A: Modest increases in heart rate (HR) and systolic blood pressure (SBP) were observed near tₘₐₓ(3-10 minutes post-dosing) at higher doses; At 10 minutes post-dosing, average ΔHR (relative to the same day baseline) were −0.3, 1.5, 3.2, 7.3, 9.4, 13.0, 13.0, and 15.4 bpm for the Treatment T1, T2, T3, T4a, T4b, T5, T6, and T7, respectively. The average ΔHR at this time point is 3.0 and 3.5 bmp for the reference Ra and Rb, respectively. At 10 minutes post-dosing, average ΔSBP were −2.4, 0, 1.9, 5.0, 4.5, 6.6, 7.5, and 12.1 mmHg for the Treatment T1, T2, T3, T4a, T4b, T5, T6, and T7, respectively. The average ΔSBP at this time point is −2.4 and −2.5 mmHg for the reference Ra and Rb, respectively. Changes in diastolic blood pressure (DBP) and respiratory rate (RR) post-dosing, on average, were minimal. No dose response relationship could be detected in DBP and RR. The greatest increases in average DBP and RR were 6.5 mmHg (45 minutes post-dosing in Treatment T3) and 1.8 bmp (60 minutes post-dosing in Treatment T5). As demonstrated in FIG. 11D, there is a decrease of DBP at 10-30 minutes. The PD profiles for IN and IM treatments were comparable.

No clinically significant changes in pharmacodynamic parameters following administration of test formulations were observed over the dose range investigated.

### Safety Evaluation

The epinephrine exposure through IN delivery was 0.73 mg and 0.85 mg per treatment on average; the total accumulated dose 2.9 mg and 3.4 mg per subject; and the highest dose per treatment 1.2 mg and 1.2 mg in subjects of cohort 1 and cohort 2, respectively. The STC exposure through IN delivery was: 0.43 mg and 0.75 mg per treatment on average; the total accumulated dose 1.7 mg and 3.0 mg per subject; and the highest dose per treatment 0.8 mg and 1.0 mg; in subjects of cohort 1 and cohort 2, respectively.

During the entire study period, all subjects were closely monitored for any incidence of adverse events by the investigators. Adverse events spontaneously reported by subjects were also recorded. All adverse events observed in this study were evaluated with different levels of relationship with study drugs by the investigators. Adverse events that were classified as “possibly related to the studied drugs”, i.e., adverse events that were identified with unknown (UK) relationship to, probably related to, or very likely related to the studied drugs by the site investigators.

A total of 345 adverse events (AEs) were reported in the study, including 158 and 187 adverse events in cohort 1 and cohort 2, respectively. Among these, 239 (69.3%) were derived from 4 vital signs (SBP, DBP, HR, and RR) measured aligned to the PK sampling scheme. Abnormal vital signs were graded by FDA Toxicity Grading Scale for Healthy Adult and Adolescent Volunteers Enrolled in Preventive Vaccine Clinical Trials guidance. They were transient in nature, fully resolved within 6 hours. The subjects did not experience any symptoms in these occurrences. Regarding adverse events, 280 occurred in 8 IN treatments (35 per IN treatment); and 65 occurred in 2 IM treatments (32.5 per IM treatment); 299 (86.7%) were classified as “definitely not related” or “unlikely related”, 42 (12.2%) were classified as “unknown” and 4 (1.1%) were classified as “probably related”; 342 (99.1%) were reported as “Recovered/Resolved”, 0 (0.0%) were reported as “Not Recovered/Not Resolved”. 3 (0.87%) were reported as “unknown”; 238 (69.0%) were classified as “Mild”; 93 (27%) were classified as “Moderate”; and 14 (4.1%) were classified as “Severe”. No AEs were reported as “serious”. No deaths or other serious adverse events (SAEs) occurred in this study.

The vital sign derived AE (VSAE) rates for IN treatments were comparable to that for IM treatments. Four (4) vital signs: systolic blood pressure (SBP), diastolic blood pressure (DBP), heart rate (HR), and respiratory rate (RR) were measured aligned to the PK sampling scheme in the study. A total of 15 measurements were taken at baseline, and 1, 3, 5, 7, 10, 15, 20, 30, 45, 60, 90, 120, 240, and 360 minutes post-dose for each 4 parameters. Abnormal vital signs were graded using the FDA guidance Toxicity Grading Scale for Healthy Adult and Adolescent Volunteers Enrolled in Preventive Vaccine Clinical Trials. FIGS. 11A-11H provide pharmacodynamic data for various embodiments of IN pharmaceutical formulations as disclosed herein versus the an IM comparator formulation.

VSAEs were reported, accounting for 69.3% f all reported AEs in this study. All VSAEs were detected from scheduled vital sign measurements aligned with the PK sampling. They were transient in nature, fully resolved within 6 hours. The subjects did not experience any symptoms. Surprisingly, at a 1.0 mg/mL concentration of STC, VSAE rates did not increase and actually were lower than some lower concentration STC doses. Surprisingly, T6 and T7, when compared to IM administration, had less incidences of bradycardia and less incidences of tachycardia. When compared to IM administration, T7 also had less incidences of nausea and vomiting.

### Intranasal Safety and Tolerability Evaluation

NOME data was collected. Data for six (6) items was collected (Nasal Irritation, mucosal edema, mucosal erythema, mucosal crusting, mucosal epistaxis, and nasal discharge) for three (3) nasal locations (Septum, Turbinates, and Nasal Floor) four (4) oropharyngeal locations (Soft palate, Tonsil/Tonsillar fossa, Base of tongue, and Posterior pharyngeal wall). This data was collected at 3 time points (baseline, 1 hr and 6 hrs) at each dosing visit (once at screening and follow-up visit) were examined. NOME nasal irritations were graded using the following scale: 0=no abnormal findings; 1A=focal nasal mucosal irritation (erythema, inflammation, or hyperemia); 1B=superficial nasal mucosal erosion; 2=moderate nasal mucosal erosion; 3=nasal mucosal ulceration; 4=nasal septum perforation. Other abnormalities were graded using the following scale: 0=none; 1=mild; 2=moderate; 3=severe.

The NOME data are summarized as follows: No Nasal irritation of grade 2 or higher was noted; no mucosal erythema, mucosal crusting, mucosal epistaxis, or nasal discharge over Grade 1 was noted. Classification per locations. Among the NOME abnormalities from the IN treatment, 28.3% were observed at the septum; 67.6% at turbinate; 2.0% at the nasal floor; 0.4% at the soft palate; 1.6% at the tonsil/tonsillar fossa; and no abnormalities at the base of the tongue or posterior pharyngeal wall. Surprisingly, no correlation between the baseline-corrected NOME rates and STC doses were observed in moderate and severe findings due to the limit data in this study.

For SRNS evaluation, all subjects were instructed to rate all four (4) symptoms in the Total Nasal Symptom Score (TNSS): 1) rhinorrhea (runny nose); 2) nasal congestion; 3) nasal itching; and 4) sneezing. Given several test formulations contain a nasal irritant STC, two (2) additional symptoms 5) nasal discomfort; and 6) facial pain/pressure were also be evaluated. Self-Reported Nasal Symptoms (SRNS) were collected at the screening visit; pre-dose, 1 hour (±15 min), and 6 hours (±20 min) post-dose in IN dosing visits 2-5, and follow-up visit. SRNS were graded using the following scale: 0=none; 1=mild (symptom clearly present but easily tolerated); 2=moderate (symptom bothersome but tolerable); 3=severe (symptom difficult to tolerate-interferes with activities). The SRNS data are summarized as follows: Rhinorrhea (no grade 3 occurrences in any of T1-T7), Nasal Congestion (no grade 3 occurrences in any of T1-T4a and T5-T7), Nasal Itching (no grade 3 occurrences in any of T1-T7), Nasal Sneezing (no grade 3 occurrences in any of T1-T7), Nasal Discomfort (only mild or moderate discomfort in T2 and T5-T7), and Facial Pain/Pressure (only mild or moderate pain/pressure in in any of T1-T4a and T5-T7). Surprisingly, any severe symptoms were reported from treatment with no or low STC doses, including T1 (STC=0), T3 (STC=0.6 mg); T4a (STC=0.8 mg); and T4b (STC=0.8 mg). Among severe symptoms, 50% were reported by a single subject in the treatment T4b in cohort 2. Only 5 SRNS symptoms were reported at the follow-up visit, suggesting that the local irritation symptoms caused by recovered at two weeks post-dosing. Total SRNS rates at follow-up visit were comparable to that at screening, suggesting that the local irritation symptoms caused the pharmaceutical formulation receded at two weeks post-dosing.

The subjects' olfactory functions were evaluated by the University of Pennsylvania Smell Identification Test (UPSIT). The UPSIT is a 40-item objective assessment of sense of olfactory function. The UPSITs were performed at screening visit; pre-dose, and 6 hours (±20 min) post-dose in IN dosing visits 2-5; and; follow-up visit. The subjects' olfactory functions were categorized using the following criteria of UPSIT score: anosmia: UPSIT score of ≤18; severe microsmia: UPSIT score 19-25; moderate microsmia: UPSIT score 26-30 in women and 26-29 in men; mild microsmia: UPSIT score 31-34 in women and 30-33 in men; normosmia: UPSIT score of more than 34 in women and 33 in men. Test formulations have no observable impact on subjects' olfactory function, as demonstrated by changes from baseline UPSIT classification.

### SUMMARY

Pharmacokinetics results are summarized as follows. Baseline levels of plasma epinephrine were detected in both IN and IM treatments. Over the dose range investigated, Cₘₐₓ and AUCs increased with increases either in STC dose (Treatment T2, T3, and T4a) or in epinephrine dose (Treatment T4b, T5, and T6). IN delivery of test formulation resulted in higher maximum concentration (Cₘₐₓ) of epinephrine than IM. The epinephrine concentration declined faster in IN delivery. Higher STC doses were associated with more rapid epinephrine absorption (tₘₐₓ). Treatment T7 (epinephrine 1.2 mg with STC 1.0 mg) appeared to have a comparable PK profile to the reference, as demonstrated by proportions of subjects with plasma epinephrine concentration greater than specific thresholds (100 pg/mL and 200 pg/mL) within 1 hour. STC absorption from the test formulations was negligible and safe when comparing to the physiological post-meal surge.

The safety and intranasal tolerability results are summarized as follows. No deaths or other serious adverse events (SAEs) occurred in this study. The subjects did not experience any symptoms in these occurrences of AEs. The vital sign derived AE (VSAE) rates for IN treatments were comparable to that for IM treatment. There was no obvious correlation between VSAE rates and STC doses. A total of 62 (18.0%) nasal AEs were reported. Nasal edema was the most commonly reported nasal AEs. No clinical significant changes from baseline in clinical laboratory parameters (hematology, clinical chemistry, and urinalysis) were observed.

The Nasal and Oropharyngeal Mucosa Exam (NOME) results are as follows. Nasal Irritation was the most commonly reported NOME findings. Most findings were mild in severity (Grade 1 or 1A). The majority of NOME abnormalities were observed at turbinate and septum. The majority of SRNS symptoms were mild (241 Grade 1, 85 Grade 2, and 10 Grade 3). Only 5 SRNS symptoms were reported at the follow-up visit, suggesting that the local irritation symptoms caused by test formulation recovered at two weeks post-dosing. Olfactory Function results are as follows. IN test formulation has no observable impact on subjects' olfactory function, as demonstrated by changes from baseline UPSIT classification. No significant differences in ECG following administration of test formulation and generic EpiPen® were observed in this study. No clinical significant changes from baseline in clinical laboratory parameters (hematology, clinical chemistry, and urinalysis) were observed.

What is claimed:

1. A pharmaceutical formulation comprising: epinephrine, or a pharmaceutically acceptable salt thereof at a concentration ranging from 1.0 mg/ml to 25.0 mg/ml; an absorption enhancer consisting of one or more bile acids or bile acid salts at a concentration ranging from 1 mg/ml to 15 mg/ml; and an aqueous carrier; wherein the pharmaceutical formulation is configured to be administered intranasally; wherein a 0.1 mL intranasal dose of the pharmaceutical formulation is configured to provide a therapeutically effective amount of epinephrine.

2. The formulation of claim 1, wherein the therapeutically effective amount of epinephrine is suitable for the treatment of a type-I hypersensitivity reaction.

3. The formulation of claim 1, wherein the absorption enhancer consists of a taurocholate salt.

4. The formulation of claim 1, wherein the absorption enhancer consists of sodium taurocholate.

5. The formulation of claim 1, wherein the pharmaceutical formulation further comprises a buffer.

6. The formulation of claim 5, wherein the buffer consists of a citrate buffer.

7. The formulation of claim 1, wherein the pharmaceutical formulation further comprises a citric acid source at a concentration that ranges from 2 mg/ml to 6 mg/ml.

8. The formulation of claim 7, wherein the citric acid source is citric acid monohydrate.

9. The formulation of claim 1, wherein the pH of the pharmaceutical formulation ranges from 2.6 to 5.0.

10. The formulation of claim 1, wherein the pharmaceutical formulation further comprises a sodium citrate source at a concentration that ranges from 6 mg/ml to 10 mg/ml.

11. The formulation of claim 10, wherein the sodium citrate source is sodium citrate dihydrate.

12. The formulation of claim 1, wherein the pharmaceutical formulation further comprises a preservative.

13. The formulation of claim 12, wherein the preservative consists of a chlorobutanol source.

14. The formulation of claim 12, wherein the preservative is at a concentration that ranges from 3.5 mg/ml to 7.5 mg/ml.

15. The formulation of claim 1, wherein the pharmaceutical formulation further comprises a tonicity agent.

16. The formulation of claim 15, wherein the tonicity agent consists of sodium chloride.

17. The formulation of claim 15, wherein the tonicity agent is at a concentration that ranges from 1 mg/ml to 3 mg/ml.

18. The formulation of claim 1, wherein the pharmaceutical formulation further comprises a metal complexing agent.

19. The formulation of claim 18, wherein the metal complexing agent consists of disodium edetate dihydrate.

20. The formulation of claim 18, wherein the metal complexing agent is at a concentration that ranges from 0.01 mg/ml to 0.03 mg/ml.

21.-69. (canceled)
